(19) United States
(12) Patent Application Publication
(10) Pub. No.: US 2024/0299394 A1
(43) Pub. Date: Sep. 12, 2024

(54) TARGETED TREATMENT FOR SKIN FRAGILITY DISEASES

(71) Applicant: University of Washington, Seattle, WA (US)

(72) Inventors: Cory L Simpson, Seattle, WA (US); Shivam Zaver, Seattle, WA (US)

(73) Assignee: University of Washington, Seattle, WA (US)

(21) Appl. No.: 18/596,363

(22) Filed: Mar. 5, 2024

Related U.S. Application Data
(60) Provisional application No. 63/450,556, filed on Mar. 7, 2023.

(51) Int. Cl. A61K 31/519; A61K 9/00; A61P 17/00

(52) U.S. Cl. CPC A61K 31/519; A61P 17/00; A61K 9/0014

(57) ABSTRACT

Various implementations described herein relate to the treatment of skin fragility diseases. Example methods and compositions described herein relate to inhibitors of the mitogen-activated protein kinase kinase (MEK or MAPK) and/or the mitogen-activated protein kinase/extracellular signal-regulated kinase (MAPK/ERK) pathway for skin fragility disease treatment. According to some implementations, the inhibitor is formulated for topical administration.

### CROSS-REFERENCE TO RELATED APPLICATION(S)

This application claims the priority of U.S. Provisional Patent Application No. 63/450,556, filed on Mar. 7, 2023, and which is incorporated by reference herein in its entirety.

### STATEMENT REGARDING FEDERALLY SPONSORED RESEARCH OR DEVELOPMENT

This invention was made with government support under Grant No. K08AR075846, awarded by the National Institute of Arthritis and Musculoskeletal and Skin Diseases. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is W149-6002US_Seq.xml. The file is 2,783 bytes, was created Mar. 4, 2024, and is being submitted electronically via Patent Center

### TECHNICAL FIELD

This application relates to methods and compositions for treating skin fragility disease, and more particularly, to inhibitors of the mitogen-activated protein kinase kinase (MEK or MAPKK) and/or the mitogen-activated protein kinase/extracellular signal-regulated kinase (MAPK/ERK) pathway for skin fragility disease treatment.

### BACKGROUND

Though advances in gene sequencing have facilitated the diagnosis of rare inherited disorders and enhanced understanding of their pathogenic mechanisms (Boyden L M, Choate K A. J Invest Dermatol. 2017; 137(5): p. e61-e65; Sarig O, Sprecher E. J Invest Dermatol. 2017; 137(5): p. e79-e82; Salik D, et al. J Eur Acad Dermatol Venereol. 2023; 37(3): p. 488-500), most genetic skin diseases lack proven, rational molecular therapies (Silverberg N. Clin Dermatol. 2020; 38(4): p. 462-466; Hill C R, Theos A. Dermatol Clin. 2019; 37(2): p. 229-239).

Mutation of the ATP2A2 gene, which encodes sarco-endoplasmic reticulum calcium ATPase 2 (SERCA2), causes Darier disease (DD) (Jacobsen N J, et al. Hum Mol Genet. 1999; 8(9): p. 1631-6; Sakuntabhai A, et al. Nat Genet. 1999; 21(3): p. 271-7), a dermatologic disorder characterized by aberrant epidermal differentiation and impaired keratinocyte adhesion via desmosomes (Hobbs R P, et al. FASEB J. 2011; 25(3): p. 990-1001; Savignac M, et al. J Invest Dermatol. 2014; 134(7): p. 1961-1970), which manifests as recurrent skin blisters, erosions, and infections (Takagi A, et al. J Dermatol. 2016; 43(3): p. 275-9; Nellen R G, et al. Hum Mutat. 2017; 38(4): p. 343-356; Dodiuk-Gad R, et al. J Eur Acad Dermatol Venereol. 2013; 27(11): p. 1405-9; Vogt K A, et al. J Am Acad Dermatol. 2015; 72(3): p. 481-4). Darier disease, an autosomal dominant disorder, has been linked to various ATP2A2 gene mutations (Jacobsen N J, et al. Hum Mol Genet. 1999; 8(9): p. 1631-6; Nellen R G, et al. Hum Mutat. 2017; 38(4): p. 343-356; Green E K, et al. J Dermatol. 2013; 40(4): p. 259-66), most of which are predicted to cause haploinsufficiency of SERCA2, a calcium channel localized to the endoplasmic reticulum (ER) (Savignac M, et al. Biochim Biophys Acta. 2011; 1813(5): p. 1111-7). SERCA2 function is essential for calcium-dependent assembly of intercellular junctions, particularly desmosomes, which maintain epidermal integrity (Hobbs R P, et al. FASEB J. 2011; 25(3): p. 990-1001; Savignac M, et al. J Invest Dermatol. 2014; 134(7): p. 1961-1970). As well, SERCA2 controls levels of cytosolic calcium, a master regulator of the terminal phase of keratinocyte differentiation (Bikle D D, et al. Expert Rev Endocrinol Metab. 2012; 7(4): p. 461-472; Elsholz F, et al. Eur J Dermatol. 2014; 24(6): p. 650-61), termed cornification, in which cells in the upper epidermis degrade their nuclei and organelles to form the flattened outermost layers of the cutaneous barrier (Matsui T, Amagai M. Int Immunol. 2015; 27(6): p. 269-80; Matsui T, et al. Proc Natl Acad Sci USA. 2021; 118(17)). In DD patients, the loss of intercellular adhesion and dysfunctional keratinocyte differentiation manifest as blistering, thick scaling, and fissuring of the skin (Takagi A, et al. J Dermatol. 2016; 43(3): p. 275-9; Savignac M, et al. Biochim Biophys Acta. 2011; 1813(5): p. 1111-7). Despite knowing the genetic etiology of this disease for more than twenty years, there are no FDA-approved therapies for DD, and no drugs that directly target the pathogenic effects of SERCA2 deficiency have advanced to prospective clinical trials (Hanna N, et al. J Cutan Med Surg. 2022; 26(3): p. 280-290).

Grover disease (GD) is a spontaneous dermatologic disorder of unknown etiology that can cause intractable pruritus, extensive vesicular skin lesions in severe cases, and cutaneous superinfection (Wang Q, et al. Clin Cosmet Investig Dermatol. 2022; 15: 1371-1376; Karray M, et al., Kaposi Varicelliform Eruption, in StatPearls. 2022: Treasure Island (FL); Gantz M, et al. J Am Acad Dermatol. 2017; 77: 952-957 e1), (Weaver J, Bergfeld W F. Arch Pathol Lab Med. 2009; 133: 1490-4; Grover R W. Arch Dermatol. 1971; 104: 26-37). However, the molecular drivers of these defects in epidermal integrity are poorly understood and there are no FDA-approved therapies for GD (Bellinato F, et al. J Dtsch Dermatol Ges. 2020; 18: 826-833; Aldana P C, Khachemoune A. Int J Dermatol. 2020; 59: 543-550). The pathologic features of GD can be indistinguishable from Darier disease (See S H C, et al. J Cutan Pathol. 2019; 46: 6-15; Chalet M, et al. Arch Dermatol. 1977; 113: 431-5). Recent sequencing of GD lesions found acquired mutation of ATP2A2 (Seli D, et al. JAMA Dermatol. 2023), the gene linked to DD (Sakuntabhai A, et al. Nat Genet. 1999; 21: 271-7; Jacobsen N J, et al. Hum Mol Genet. 1999; 8: 1631-6).

### BRIEF DESCRIPTION OF THE DRAWINGS

Some of the drawings submitted herewith may be better understood in color. Applicant considers the color versions of the drawings as part of the original submission and reserves the right to present color images of the drawings in later proceedings.

FIG. 1 illustrates an example environment for treating a subject suffering from a skin fragility disease.

FIGS. 2A-2F show that a loss of sarco-endoplasmic reticulum calcium ATPase 2 (SERCA2) in human keratinocytes impairs cytosolic calcium handling and reduces intercellular adhesive strength. FIG. 2A illustrates an example immunoblot of SERCA2 in lysates from ATPase SERCA2 (ATP2A2) wild-type (WT, +/+), heterozygous (HET, +/−), and homozygous knockout (KO, −/−) cells. hTERT-immortalized human epidermal keratinocytes (THEKs) were differentiated in E-medium for 72 hours before lysate harvesting; data represent 3 independent experiments; and β-actin is a loading control. FIG. 2B illustrates example immunofluorescence of SERCA2 (green) in WT, HET, and KO THEKs. Images are representative of 14 independent high-powered fields (HPF) per genotype. Hoechst (blue) stains nuclei, and scale bar is equal to 10 μm. FIG. 2C illustrates example mechanical dissociation of monolayers from control (+/+), HET (+/−), and KO (−/−) cells grown in 1.3 mM CaCl₂ for 72 hours prior to using dispase to release intact monolayers; representative images of fragmented monolayers transferred into 6-well cell culture plates after mechanical stress are shown. FIG. 2D includes example graphs that display mean±SD of the number of epithelial fragments with data plotted for N=3 biological replicates. P values are from 1-way ANOVA with Dunnett's adjustment for multiple comparisons. FIG. 2E illustrates example change (Δ) in intensity of GCaMP in control (+/+), HET (+/−), and KO (−/−) THEKs from baseline at t=0 seconds with addition of 1 mM CaCl₂ at t=60 seconds (arrow). Data is plotted as mean±SEM from N=3 independent experiments per genotype. FIG. 2F illustrates examples of fluorescence images of GCaMP in WT, HET, and KO cells in low CaCl₂ (top) or high CaCl₂ (bottom). Scale bar is equal to 10 μm.

FIG. 3 shows that depletion of SERCA2 does not alter proliferation of keratinocytes. ATP2A2 control (+/+), HET (+/−), and KO (−/−) keratinocyte growth was measured over 48 hours. Graph displays the mean+/−SD of the percent confluency across 25 non-overlapping microscopic fields at each time point for N=6 biological replicates per genotype; P-value calculated by simple linear regression to test if the slopes (growth rate) of the best-fit lines for each genotype were significantly different.

FIGS. 4A-4C show that keratinocytes lacking SERCA2 exhibit transcriptomic alterations in mediators of epidermal adhesion and differentiation. FIG. 4A includes example results from bulk RNA sequencing of SERCA2 HET versus control cells. Volcano plot depicts log 2 fold-changes of genes significantly downregulated (diamond) and upregulated (chevron) with cutoff (dashed line) 0.05 for adjusted P value. FIG. 4B includes example gene ontology (GO) analysis of transcripts, which are significantly altered (adjusted P s 0.05) in HET cells and reveal upregulation (chevron) in genes controlling ER stress and growth factor signaling and downregulation (diamond) of genes controlling epidermal differentiation and antiviral response. FIG. 4C illustrates example reverse transcription quantitative PCR (RT-qPCR) results of mRNA transcripts from control (+/+), HET (+/−), or KO (−/−) THEKs that were differentiated in E-medium for 24 hours. Graphs display mean±SD with plotted values from N=4 biological replicates. P values are from 1-way ANOVA with Dunnett's adjustment for multiple comparisons. DSG1, desmoglein 1; KRT, keratin.

FIGS. 5A-5E show that proteomics analysis of SERCA2-deficient keratinocytes reveals alterations in key regulators of tissue integrity and skin barrier formation. FIG. 5A illustrates example label-free quantitative (LFQ) mass spectrometry-based comparison of the proteomes of control versus HET THEKs differentiated in E-medium for 72 hours. Proteins indicated by triangles were absent or detected in lower abundance in HET cells; proteins indicated by diamonds were detected in higher abundance in control THEKs; proteins indicated by chevrons were detected in similar abundance in control and HET cells; raw values and statistical analysis summarized in Table 1, as illustrated in the First Experimental Example. FIG. 5B illustrates example immunoblotting of keratin (KRT) 10 and KRT14 (on a separate blot run in parallel) in lysates from control, HET, and KO THEKs differentiated in E-medium for 72 hours. Data is representative of 5 independent experiments, and β-actin is a loading control. FIG. 5C illustrates example H&E-stained tissue cross sections from organotypic cultures grown from control, HET, or KO THEKs. HET and KO cultures display aberrant differentiation with the upper granular layers exhibiting vacuolization and loss of cohesion along with dyskeratotic cells having deeply pink cytoplasm and condensed nuclei indicative of aberrant cornification (insets). Scale bar is equal to 100 μm, and insets correspond to an original magnification of 40×. FIG. 5D illustrates example immunostaining of KRT10 and phosphorylated extracellular signal-regulated kinase (pERK) in tissue cross sections from organotypic cultures of control or HET THEKs. Scale bar is equal to 50 μm, and dashed line marks bottom of epidermis. FIG. 5E illustrates example quantification of tissue immunostaining of KRT10 (relative to Hoechst) and pERK (relative to total ERK). Data is shown as a box plot of the 25th-75th percentile with a line at the median from N≥26 nonoverlapping high-powered fields (HPF) per condition from 4 experiments using 2 independent HET lines. Control mean is normalized to 1, and P values are from 2-tailed unpaired Student's t test. DSP, desmoplakin; JUP, plakoglobin; IVL, involucrin; TGM1, transglutaminase 1; PKP, plakophilin; p-, phosphorylated.

FIG. 6 shows that desmosomal protein localization is impaired in SERCA2-deficient keratinocytes: ATP2A2 control (+/+) and HET (+/−) cells were grown to confluency, then were switched into E-medium to induce differentiation and assembly of intercellular junctions. Immunostaining of desmosomal components after 24 hours showed markedly reduced concentration of desmosomal cadherins (DSG1, DSG2, DSG3) and plakoglobin at sites of cell-cell contact in HET cells compared to control keratinocytes, which exhibited robust assembly of desmosomes. Images are representative of N=32 images from 2 experimental replicates, and scale bar is equal to 10 μm.

FIGS. 7A-7C show that SERCA2-deficient keratinocytes exhibit aberrant ERK signaling and reduced KRT10 expression. FIG. 7A illustrates example immunoblotting of pERK that shows prolonged ERK activation in HET cells compared to controls. ATP2A2 control (+/+) and HET (+/−) cells were grown to confluency, then were switched into medium lacking growth supplements and calcium for 4 hours. Calcium was added to reach 1.3 mM, and lysates were collected at baseline and after up to 30 minutes. β-actin is a loading control. Blots are representative of 2 independent experiments. FIGS. 7B and 7C illustrate example immunostaining of fixed cells for pERK or KRT10. ATP2A2 control (+/+) and HET (+/−) cells were seeded at confluency, then were switched into E-medium to induce differentiation for 24 hours. Hoechst stains nuclei, and scale bar is equal to 10 μm. Fluorescence intensity data are shown as a box plot of the 25th-75th percentile with a line at the median from N≥47 non-overlapping high-powered microscopic fields per condition from 2 independent experiments. Control mean is normalized to 1, and P values are from two-tailed unpaired Student's t-test.

FIGS. 8A-8F show that chemical inhibition of SERCA2 in keratinocytes and organotypic epidermis replicates features of Darier Disease (DD) pathology and induces ERK activation. FIG. 8A illustrated example mechanical dissociation of confluent monolayers from THEKs cultured in 1.3 mM CaCl₂ with DMSO or 1 μM thapsigargin (TG) for 24 hours. Representative images of fragmented monolayers transferred into 6-well cell culture plates are shown. FIG. 8B includes example graphs that display mean±SD of the number of fragments with data points for N=6 biological replicates. P value is from 2-tailed unpaired Student's t test. FIG. 8C illustrates example H&E-stained tissue cross sections of organotypic cultures of NHEKs treated for 48 hours with DMSO or 1 μM TG. Inset shows separation between basal and suprabasal layers in TG-treated cultures. Scale bar=100 μm. FIG. 8D illustrates example quantification of retained nuclei in cornified layers. Data is shown as a box plot of the 25th-75th percentile with a line at the median from N≥45 nonoverlapping high-powered fields (HPF) from 3 biological replicates. P value is from a 2-tailed unpaired Student's t test; (right) H&E-stained tissue cross section from a TG-treated culture shows retention of nuclei in cornified layers (magnified in inset). Scale bar is equal to 100 μm, and insets correspond to an original magnification of 40×. FIG. 8E illustrates example immunostaining of KRT10 and pERK in tissue cross sections from organotypic cultures of NHEKs after 48 hours of DMSO or TG treatment. Scale bar is equal to 50 μm, and dashed line marks bottom of epidermis. FIG. 8F illustrates example quantification of tissue immunostaining of KRT10 (relative to Hoechst) and pERK (relative to total ERK) in organotypic cultures of NHEKs treated with DMSO or TG. Data is shown as a box plot of the 25^{th}-75^{th} percentile with a line at the median from N≥98 nonoverlapping HPF per condition from 4 biological replicates. Control mean is normalized to 1, and P values are from 2-tailed unpaired Student's t test.

FIGS. 9A-9E show that biopsies of DD exhibit mislocalization of cadherins, loss of keratin 10, and elevated ERK activation. FIG. 9A illustrate example H&E-stained cross sections of punch biopsies from skin of control donors versus patients with DD, which demonstrate loss of keratinocyte cohesion (acantholysis) and aberrant cornification (dyskeratosis) with retention of nuclei in cornified layers (magnified in inset, which corresponds to an original magnification of 40×). Scale bar is 100 μm. FIG. 9B illustrates example quantification of immunostaining of KRT10 (relative to Hoechst) and pERK (relative to total ERK) in cross sections of control skin versus in lesional or nonlesional portions of DD biopsies. Graph depicts mean±SD from N≥20 nonoverlapping high-powered fields (HPF) per group from 5 control and 5 DD patients. Control mean is normalized to 1. P values are from 1-way ANOVA with Dunnett's adjustment for multiple comparisons. FIG. 9C illustrates example immunostaining of KRT10, FIG. 9D illustrates example immunostaining of DSG1, and FIG. 9E illustrates example immunostaining of pERK in tissue cross sections from patient biopsies. Images are from 2 control and 2 DD patients representative of 5 patients in each group. Scale bar is 50 μm, and the dashed line marks the bottom of epidermis.

FIGS. 10A-10G show that keratin expression and adhesive integrity in SERCA2-deficient keratinocytes are rescued by MEK inhibitors. FIG. 10A illustrates example RT-qPCR data of transcripts from control or HET cells that were differentiated in E-medium±MEK inhibitors for 24 hours. Graphs show mean±SD from N=4 replicates. P values are from ANOVA (Brown-Forsythe) with Dunnett's T3 adjustment for multiple comparisons. FIG. 10B illustrates example immunostaining KRT10 (and Hoechst) in control or HET cells treated with DMSO or 1 μM trametinib (Tram) for 48 hours. Images represent 2 independent experiments using 2 control and 2 SERCA2-deficient lines. Scale bar is equal to 50 μm. FIG. 10C illustrates example quantification of KRT10 immunostaining in control versus SERCA2-deficient cells treated with DMSO or 1 μM Tram for 24 hours. Data is shown as a box plot of 25th-75th percentile with line at the median from N≥21 nonoverlapping high-powered fields (HPF) per group. Control mean is normalized to 1. P values are from 1-way ANOVA with Tukey's adjustment for multiple comparisons. FIG. 10D illustrates an example immunoblot of KRT10 and KRT14 (β-actin loading control) in lysates from control versus HET cells treated with DMSO, 1 μM dabrafenib (Dab), or MEK inhibitors (1 μM Tram; 10 μM U0126; 10 μM PD184352 (PD184); 20 μM PD98059 (PD980)). Data represent 2 independent experiments. FIG. 10E illustrates example quantification of fragments of HET and KO monolayers treated with DMSO or 1 μM trametinib for 48 hours. Graphs display mean±SD for N=3 replicates. P values are from 1-way ANOVA with Tukey's adjustment for multiple comparisons. FIG. 10F includes example images of 6-well culture plates containing fragmented monolayers of control or HET cells treated with DMSO or 20 μM PD98059 (PD980) for 24 hours. FIG. 10G illustrates example quantification of fragments of control, HET, or KO monolayers treated with DMSO or 20 μM PD98059 (PD980) for 24 hours. Graphs display mean±SD for N=3 replicates. P values are from 1-way ANOVA with Dunnett's adjustment for multiple comparisons.

FIGS. 11A-11F show that MEK inhibitors promote keratinocyte cohesion and mitigate epidermal tissue disruption from SERCA2 inhibition. FIG. 11A illustrate example immunostaining of KRT10 (and Hoechst) in THEKs treated with DMSO, 1 μM TG, or 1 μM TG plus 1 μM Tram (TG+Tram) for 48 hours. Scale bar is equal to 50 μm. FIG. 11B illustrates example quantification of KRT10 immunostaining in THEKs treated with DMSO, TG, or TG plus Tram. Data is shown as a box plot of the 25th-75th percentile with a line at the median from N≥31 nonoverlapping high-powered fields (HPF) from 2 independent THEK lines for both control and SERCA2-deficient cells; Control mean normalized to 1, and P values are from 1-way ANOVA with Tukey's adjustment for multiple comparisons. FIG. 11C include example representative images of fragmented monolayers transferred into 6-well cell culture plates for NHEKs treated with DMSO versus 1 μM TG alone or with 1 μM dabrafenib versus an MEK inhibitor (1 μM Tram, 10 μM U0126, or 20 μM PD98059 (PD980)) for 24 hours. FIG. 11D illustrates example quantification of epithelial fragments of NHEK monolayers. Bar graphs display mean±SD with data points for N=6 biological replicates. P values are from 1-way ANOVA with Tukey's adjustment for multiple comparisons. FIG. 11E illustrate example H&E-stained tissue cross sections of organotypic cultures of NHEKs treated with DMSO, 1 μM TG, or 1 μM TG plus 20 μM PD98059 (PD980), the latter displaying improved keratinocyte cohesion and normalization of cornification. Scale bar is equal to 100 μm. FIG. 11F illustrates example quantification of retained nuclei in cornified layers of organotypic cultures treated with the indicated inhibitors for 48 hours. Graph displays mean±SD with plotted values averaged from ≥49 nonoverlapping HPF per condition from N=4 biological replicates. P values are from 1-way ANOVA with Tukey's adjustment for multiple comparisons.

FIGS. 12A-12E show that sustained B-RAF blockade paradoxically activates ERK in human epidermal keratinocytes. FIG. 12A illustrates an example immunoblot of total and phosphorylated ERK (pERK) in lysates from NHEKs treated with dabrafenib (Dab, 1 μM) or vemurafenib (Vem, 10 μM)+/−trametinib (Tram, 1 μM) for 24 hours in E-medium. GAPDH is a loading control. FIG. 12B includes example bar graphs that display the mean±SD of the intensity of pERK (normalized to total ERK) with individual data points plotted for N=2 (Dab) or N=4 (Vem) independent experiments. FIG. 12C illustrates example representative confocal fluorescence microscopy images of NHEKs transduced with the ERK biosensor (ERK-KTR) linked to the green mClover fluorophore. Cells were treated with the indicated compounds for 24 h in medium containing 1.2 mM calcium. Scale bar is equal to 10 μm. FIG. 12D includes an example diagram of the ERK biosensor, which is primarily localized in the nucleus when ERK is inactive vs. in the cytoplasm when ERK is active. An ERK activity index is calculated as the cytoplasmic to nuclear fluorescence intensity ratio. FIG. 12E illustrates example ERK activity data for each treatment group depicted as a box plot of the 25th-75th percentile with a line at the median from N≥66 cells per condition from 2 biological replicates. Mean ERK activity of DMSO was normalized to 1. P-values are from one-way ANOVA using the Bonferroni adjustment for multiple comparisons.

FIGS. 13A and 13B show that B-RAF inhibition disrupts desmosomal protein localization in epidermal keratinocytes. FIG. 13A includes an example immunoblot of pan-cadherins (Pan-Cad), desmosomal cadherins (DSG1, DSG2, DSG3) and plakoglobin in lysates from NHEKs treated with Dab or Ven+/−Tram for 24 hours in E-medium. β-actin is a loading control. FIG. 13B illustrates example immunofluorescence of plakoglobin in NHEKs treated with the indicated compounds for 24 hours in E-medium.

FIGS. 14A-14D show that MEK suppression reverses B-RAF inhibitor-induced weakening of intercellular adhesion. FIG. 14A illustrates an example mechanical dissociation assay of confluent monolayers from NHEKs cultured in E-medium with the indicated compounds for 24 hours. Representative images of fragmented monolayers transferred into 6-well cell culture plates are shown. FIG. 14B includes an example bar graph that displays the mean±SD of the number of fragments with individual data points plotted for N=6 (Dab) or N=4 (Vem) biological replicates. P-values are from one-way ANOVA with Dunnett adjustment for multiple comparisons to control cells. FIG. 14C includes an example bar graph that displays the mean+SD of the number of fragments with individual data points plotted for N=6 biological replicates. P-values are from one-way ANOVA with Dunnett adjustment for multiple comparisons to control cells. FIG. 14D includes an example diagram that depicts the regulation of desmosome stability by the MAP kinase pathway. B-RAF inhibitors paradoxically activate C-RAF, and MEK downstream, which inhibits desmosome stability, an effect overcome by MEK inhibitors.

FIGS. 15A and 15B show that B-RAF inhibitors reversibly disrupt cell-cell junctions in organotypic human epidermis. FIG. 15A illustrates example immunostaining of KRT10, DSG1, and plakoglobin in tissue cross-sections from organotypic cultures of NHEKs after 48 hours of treatment with DMSO, Dab, or Dab plus Tram; Hoechst stains nuclei (Nuc). FIG. 15B illustrates example immunostaining of KRT10, DSG1, and plakoglobin in tissue cross-sections from organotypic cultures of NHEKs after 48 hours of treatment with DMSO, Vein, or Vein plus Tram. Hoechst stains nuclei (Nuc).

FIGS. 16A-16D show that biopsies of Grover disease exhibit ERK hyperactivation along with desmosomal disruption. FIG. 16A illustrates example H&E-stained cross-sections of punch biopsies from the skin of a control donor versus a patient with Grover disease, which demonstrates aberrant cornification (dyskeratosis) with retention of nuclei in the cornified layers and loss of keratinocyte cohesion (magnified in inset). Scale bar is equal to 100 μm. FIG. 16B illustrates example immunostaining of DSG1 and plakoglobin in tissue cross-sections from patient biopsies; images shown are from 2 control and 2 Grover disease patients and are representative of N=15 patients in each group. FIG. 16C illustrates example immunostaining of pERK and Hoechst to stain nuclei (Nuc) in tissue cross-sections from patient biopsies. Images shown are from 2 control and 2 Grover disease patients and are representative of N=15 patients in each group. FIG. 16D illustrates example quantification of immunostaining pERK (relative to nuclear stain) in cross-sections of control skin versus in lesional or non-lesional portions of Grover disease biopsies, and pERK intensity data for each group are depicted as a box plot of the 25th-75th percentile with a line at the median from N=8 control vs. N=8 Grover disease biopsies. Control mean was normalized to 1, and P-values are from one-way ANOVA using the Tukey adjustment for multiple comparisons.

### DETAILED DESCRIPTION

While systemic retinoids have been used off-label for treatment of Darier Disease (DD) (Christophersen J, et al. Acta Derm Venereol. 1992; 72(2): p. 150-2; Dicken C H, et al. J Am Acad Dermatol. 1982; 6(4 Pt 2 Suppl): p. 721-6), their long-term toxicity and highly teratogenic nature limit utilization in patients suffering from this lifelong disorder (Zaenglein A L, et al. Pediatr Dermatol. 2021; 38(1): p. 164-180; Doolan B J, et al. Clin Exp Dermatol. 2022; 47(12): p. 2273-2276). Similarly, topical corticosteroids and antimicrobials can treat secondary inflammation and infection in localized lesions (Hanna N, et al. J Cutan Med Surg. 2022; 26(3): p. 280-290), but are not specifically aimed at the underlying disease pathophysiology and exhibit limited efficacy, especially in widespread disease that can lead to serious illness (Dodiuk-Gad R, et al. J Eur Acad Dermatol Venereol. 2013; 27(11): p. 1405-9; Vogt K A, et al. J Am Acad Dermatol. 2015; 72(3): p. 481-4; Ashok Kumar P, et al. Cureus. 2020; 12(5): p. e8133). Unfortunately, targeted ablation of the Atp2a2 gene in mice did not replicate DD pathology, but instead led to increased age-related keratinocyte carcinomas (Prasad V, et al. Cancer Res. 2005; 65(19): p. 8655-61; Toki H, et al. Biochem Biophys Res Commun. 2016; 476(4): p. 175-182), a risk not borne out in patients.

Similarly, current therapy of GD is based on case series and expert opinion as it lacks any treatment proven in prospective trials (Bellinato F, et al. J Dtsch Dermatol Ges. 2020; 18: 826-833; Aldana P C, Khachemoune A. Int J Dermatol. 2020; 59: 543-550; Sousou J M, et al. Cureus. 2022; 14: e24082). Retinoids, which regulate the differentiation of keratinocytes, have been used in observational studies for G D (Bellinato F, et al. J Dtsch Dermatol Ges. 2020; 18: 826-833; Aldana P C, Khachemoune A. Int J Dermatol. 2020; 59: 543-550; Pasmatzi E, et al. Dermatol Online J. 2019; 25; Hrin M L, et al. J Cutan Med Surg. 2023: 12034754231211567; Helfman R J. J Am Acad Dermatol. 1985; 12: 981-4), but they carry various risks as described above (Zaenglein A L, et al. Pediatr Dermatol. 2021; 38:164-180; Doolan B J, et al. Clin Exp Dermatol. 2022; 47: 2273-2276). Other conventional, but off-label, therapies for GD include topical corticosteroids and repeated narrow-band ultraviolet light treatment, which is burdensome for patients (Bellinato F, et al. J Dtsch Dermatol Ges. 2020; 18: 826-833; Aldana P C, Khachemoune A. Int J Dermatol. 2020; 59: 543-550). More recently, dupilumab has been reported as an off-label treatment for refractory GD (Shelton E, et al. JAAD Case Rep. 2022; 22: 31-33; Barei F, et al. Dermatol Ther. 2022; 35: e15429; Butler D C, et al. JAMA Dermatol. 2021; 157: 353-356), though its blockade of interleukin-4 and -13 receptors may target secondary pruritus and inflammation rather than the primary pathogenic drivers of GD.

Both Grover- and Darier-like eruptions have also been reported as common cutaneous toxicities of systemic therapy with B-RAF inhibitors including dabrafenib and vemurafenib (Anforth R, et al. Lancet Oncol. 2013; 14: e11-8; Chu E Y, et al. J Am Acad Dermatol. 2012; 67: 1265-72), which are utilized in the treatment of proto-oncogene B-Rapidly Accelerated Fibrosarcoma (BRAF) mutant cancers such as melanoma (Fu L, et al. J Med Chem. 2022; 65: 13561-13573; Flaherty K T, et al. N Engl J Med. 2012; 367: 107-14; Flaherty K T, et al. N Engl J Med. 2012; 367: 1694-703; Falchook G S, et al. Lancet Oncol. 2012; 13: 782-9). Prolonged B-RAF inhibition has been shown to induce paradoxical activation of mitogen-activated protein kinase kinase (MEK, also referred to as MAPKK) (Poulikakos P I, et al. Nature. 2010; 464: 427-30; Lacouture M E, et al. Cancer Discov. 2021; 11: 2158-2167), which operates downstream of RAF in the mitogen-activated protein (MAP) kinase pathway, but how this signaling aberration induces the pathologic features of GD in human epidermal keratinocytes remains unknown. In a recent study, Darier disease was linked to MEK overactivation (Zaver S A, et al. JCI Insight. 2023), which indicates that MEK hyperactivity may fuel GD pathogenesis. Consistent with this, retrospective analysis of patients co-treated with MEK inhibitors instead of B-RAF inhibitor monotherapy failed to develop GD as a side effect (Carlos G, et al. JAMA Dermatol. 2015; 151: 1103-9).

Early studies of GD using histology and electron microscopy identified defects in desmosomes (Grover R W. Arch Dermatol. 1971; 104: 26-37; Hashimoto K, et al. J Cutan Pathol. 1995; 22: 488-501), cell-cell junctions that are essential to maintain epidermal tissue integrity (Simpson C L, et al. Nat Rev Mol Cell Biol. 2011; 12: 565-80). Given numerous studies connecting the MAP kinase pathway to stability of desmosomes (Valenzuela-Iglesias A, et al. Mol Cancer Res. 2019; 17: 1195-1206; Kam C Y, et al. J Cell Biol. 2018; 217: 3219-3235; Harmon R M, et al. J Clin Invest. 2013; 123: 1556-70; Getsios S, et al. J Cell Biol. 2009; 185: 1243-58; Egu D T, et al. Front Immunol. 2019; 10: 2883; Hutz K, et al. Mol Carcinog. 2017; 56: 1884-1895; Wei Z, et al. Oncotarget. 2016; 7: 29429-39; Spindler V, et al. J Invest Dermatol. 2014; 134: 1655-1664), the effect of MEK and downstream ERK overactivation in GD on intercellular adhesion was examined in the Experimental Examples disclosed herein. As described herein, drug-induced GD was modeled in human keratinocytes and in 3D organotypic epidermis (Simpson C L, et al. Methods Mol Biol. 2010; 585: 127-46) using B-RAF inhibitors, which weakened cell-cell adhesion in an ERK-dependent manner. Epidermal ERK hyper-activation was found in GD skin biopsies, indicating that MEK inhibition could serve as a novel targeted therapy for spontaneous GD.

Various implementations described herein relate to methods and compositions for treatment of skin fragility diseases. As used herein, “skin fragility diseases” refers to skin blistering diseases and dermatologic disorders associated with dysregulation of sarcoplasmic/endoplasmic reticulum Ca2+ transporting 2 (SERCA2), ATPase SERCA2 (ATP2A2), ATPase secretory pathway Ca2+ transporting 1 (ATP2C1), secretory pathway Ca2+ ATPase pump type 1 (SPCA1), MEK, extracellular signal-regulated kinase (ERK), phosphorylated ERK (pERK), proto-oncogene B-Rapidly Accelerated Fibrosarcoma (B-RAF), proto-oncogene C-RAF (C-RAF), a keratin (e.g., KRT1, KRT2, KRT5, KRT10, KRT14, KRT16, KRT17), or a desmosome component (e.g., desmoglein (DSG) 1, DSG2, DSG3, DSG4, desmocollin (DSC) 1, DSC2, DSC 3, plakoglobin, desmoplakin, plakophilin, or another desmosome component). Examples of skin fragility disease include Darier disease, Grover disease, Hailey-Hailey disease, pemphigus, epidermolytic ichthyosis, epidermolysis bullosa simplex, keratoderma, palmoplantar keratoderma, or pachyonychia congenita.

Various implementations of the present disclosure include inhibitors of the mitogen-activated kinase (MAPK)/ERK pathway for treatment of skin fragility diseases. In particular implementations, MAPK/ERK pathway inhibitors include MEK inhibitors that reduce hyperactivation of the MAPK/ERK pathway. Particular implementations include topical formulations of compositions described herein.

Various implementations of the present disclosure will now be described with reference to the accompanying figures.

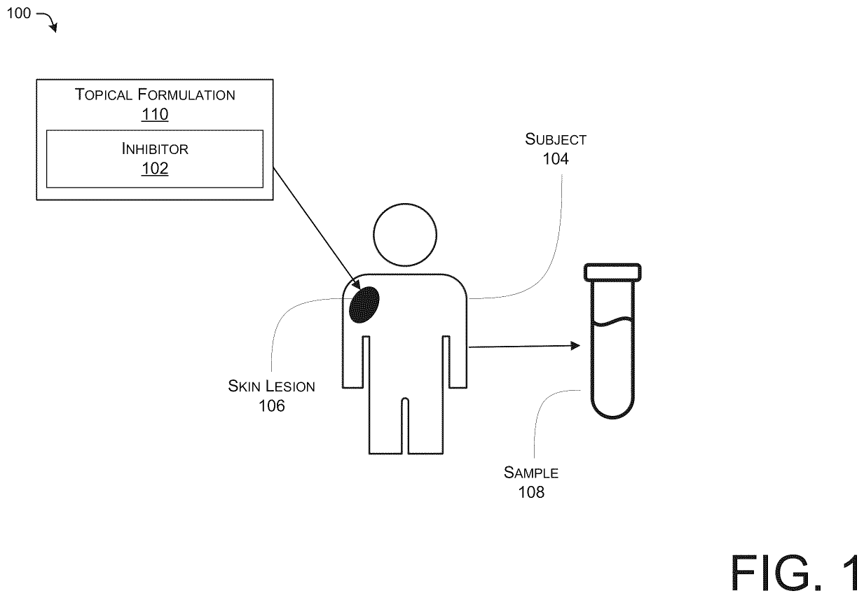

FIG. 1 illustrates an example environment 100 for administration of a treatment, such as an inhibitor 102, to a subject 104 suffering from a skin fragility disease. The inhibitor 102, in various examples, is a MEK inhibitor or a MAPK/ERK pathway inhibitor. The subject 104, in various examples, is a human or other animal. In some cases, the skin fragility disease is a skin blistering disease. For example, the skin fragility disease may include Darier disease, Grover disease, Hailey-Hailey disease, pemphigus, epidermolytic ichthyosis, epidermolysis bullosa simplex, keratoderma, palmoplantar keratoderma, or pachyonychia congenita. In some examples, the skin fragility disease is associated with dysregulation of at least one of SERCA2, ATP2A2, ATP2C1, SPCA1, MEK, ERK, pERK, B-RAF, C-RAF, a keratin (e.g., KRT1, KRT2, KRT5, KRT10, KRT14, KRT16, KRT17), or a desmosome component (e.g., DSG1, DSG2, DSG3, DSG4, DSC1, DSC2, DSC3, plakoglobin, desmoplakin, plakophilin, or another desmosome component). In some examples, the skin fragility disease is associated with hyperactivation of the MAPK/ERK pathway. In various cases, a skin lesion 106 associated with the skin fragility disease is present on the subject 104.

In various examples, at least one care provider may diagnose the subject 104 with the skin fragility disease. In particular implementations, detection of one or more markers associated with the skin fragility disease is useful in diagnosis and/or prognosis of a condition of the subject 104. The term “diagnosis”, as used herein, refers to evaluation of the presence or properties of pathological states or lack thereof. As used herein, prognosis of the condition of the 13hosphot 104 can refer to evaluating indicia of the skin fragility disease at a given time point and comparing it to the same indicia taken at an earlier time point, wherein the comparison is indicative of a progression of the skin fragility disease in the subject 104.

In some examples, the care provider(s) (e.g., a clinician) may diagnose the subject 104 based on a clinical evaluation, a family history, or a genetic test. In some examples, the care provider(s) may perform a biopsy to collect a sample 108 from the subject 104. The sample 108 may be collected from the skin lesion 106. The care provider(s) (e.g., a medical technologist) may perform analysis on the sample 108 to determine a level or an activity level of a marker associated with the skin fragility disease. The marker, for instance, may be SERCA2, ATP2A2, ATP2C1, SPCA1, KRT1, KRT2, KRT5, KRT10, KRT14, KRT16, KRT17, DSG1, DSG2, DSG3, DSG4, DSC1, DSC2, DSC3, plakoglobin, desmoplakin, plakophilin, ERK, pERK, B-RAF, C-RAF, or MEK. In some examples, determining a level of a gene includes determining a presence or a level of a variant of the gene (i.e., a gene that includes a mutation).

In various cases, the care provider(s) may compare the level or the activity level of the marker to a reference level. Reference levels may be obtained from a population of subjects without skin fragility symptoms or diseases. For instance, the reference level can be based on any mathematical or statistical formula useful and known in the art for arriving at a meaningful aggregate reference level from a collection of individual data points, such as a mean, median, median of the mean, etc. In particular implementations, a reference level may be obtained from a population of subjects including those that have a skin fragility disease.

In particular implementations, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.10, 0.05, or 0.01. In particular implementations, a sample value is “comparable to” a reference level derived from a normal control population if the sample value and the reference level are not statistically significantly different.

Currently, treatment options are limited for skin fragility diseases. Treatment options, including systemic retinoids, topical corticosteroids, and antimicrobials, suffer from long-term toxicity, limited efficacy, and other challenges. Systemic retinoids include retinol, tretinoin, isotretinoin, adapalene, alitretinoin, acitretin, etretinate, bexarotene, tazarotene, and trifarotene. Topical corticosteroids include hydrocortisone (e.g., hydrocortisone acetate, hydrocortisone 17-butyrate), diflucortolone valerate, triamcinolone acetonide, fluticasone, mometasone furoate, betamethasone (e.g., betamethasone propionate, betamethasone valerate), methylprednisolone aceponate, desonide, clobetasol propionate, fluocinonide, fluticasone, and halobetasol. Antimicrobials include antibiotics (e.g., dicloxacillin, erythromycin, aminoglycosides, and tetracycline) and antiseptics (e.g., chlorhexidine and sodium hypochlorite). In various examples, it may be beneficial to administer a treatment that reduces or eliminates the symptoms of the skin fragility disease. According to the present disclosure, hyperactivation of the MAPK/ERK pathway is associated with dysregulation of cell differentiation and adhesion. In various implementations, inhibiting the MAPK/ERK pathway may reduce or eliminate the symptoms of the skin fragility disease by reducing ERK activity or phosphorylation.

In some examples, the care provider(s) may prescribe and/or administer, to the subject 104, a topical formulation 110 that includes the inhibitor 102 for treatment of the skin fragility disease. According to various implementations of the present disclosure, the inhibitor 102 reduces hyperactivation of the MAPK/ERK pathway. In particular implementations, any drug that reduces the expression or activation of C-RAF, MEK, or the MAPK/ERK pathway may be administered. In some examples, the inhibitor 102 is a MEK inhibitor. Examples of MEK inhibitors, in some cases, include trametinib, cobimetinib, binimetinib, refametinib, selumetinib, U0126, PD98059, or PD184352. The drug, in various implementations, is a pharmaceutical drug or a therapeutic agent that is administered to the subject 104 for the purpose of diminishing or eliminating signs or symptoms of the pathology. The term “topical formulation” refers to any formulation which is pharmaceutically acceptable for external administration of a compound by application of the formulation to the epidermis.

In some examples, the inhibitor 102 is formulated in the form of an ointment, a paste, a cream, a lotion, a gel, a powder, a solution, a spray, an inhalant, a wound dressing (e.g., a bandage, a patch), a suspension, an emulsion, a crystalline form, an oil, a plaster, a liposome, a microemulsion, or a buffered solution. In various examples, the inhibitor 102 may be a shampoo or another appropriate topical formulation.

An ointment may be a homogeneous, viscous, semi-solid preparation, most commonly a greasy, thick oil (oil 80%-water 20%) with a high viscosity. Ointments have a water number, which is the maximum quantity of water that 100 g of a base can contain at 20° C.

A paste may include three agents: oil, water, and powder, one of which includes a therapeutic agent. Pastes can be an ointment in which a powder is suspended.

A cream may be an emulsion of oil and water in approximately equal proportions. Creams are thicker than lotions and maintain their shape when removed from a container. In various implementations, a cream may include saturated or unsaturated fatty acids such as stearic acid, palmitic acid, oleic acid, palmito-oleic acid, cetyl or oleyl alcohols. A cream may also include a non-ionic surfactant, for example, polyoxy-40-stearate.

A lotion may include oil, water, and powder, but can have additional components (e.g., alcohol to hold the emulsion together) and often has a lower viscosity than a paste.

A gel may be a substantially dilute cross-linked system, which exhibits no flow when in the steady-state. Most gels are liquid, however they behave more like solids due to the three-dimensional cross-linked network within the liquid. Gels can have properties ranging from soft and weak to hard and tough.

Topical formulations disclosed herein (e.g., the topical formulation 110) can include components, such as animal and vegetable fats, oils, waxes, paraffins, starch, tragacanth, cellulose derivatives, polyethylene glycols, silicones, bentonites, silicic acid, talc and zinc oxide, or mixtures thereof. In various implementations, topical formulations may include thickening agents, surfactants, organic solvents, tonicity modifiers, pharmaceutically acceptable carriers (i.e., excipients), penetration enhancing agents, and therapeutically active ingredients.

In various implementations, topical formulations can be prepared using thickening agents, such as carboxymethylcellulose sodium, sodium starch glycollate type C, or Carbo-mers such as Carbopol® (Lubrizol Advanced Materials, Inc. Cleveland, OH, USA) 934, 980, 981, 1382, 5984, or 2984. In various implementations, topical formulations can be prepared using surfactants, such as Pluronic® (BASF Corporation, Mount Olive, NJ, USA) co-polymers, such as Pluronic® F-127, and/or a Pluronic® co-polymer having the formula:

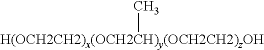

(wherein x is 2 to 130, y is 15 to 70, and z is 2 to 130), or H[OCH₂CH2]₄₉[OCHCH₂]₆₇[OCH₂CH₂]₄₉OH; propyl glycol, polypropylene glycol (PPG) stearyl ethers, such as PPG ethers of stearyl alcohol including PPG-20 methyl glucose ether distearate, PPG-15 Stearyl Ether, and PPG-11 Stearyl Ether.

In various implementations, topical formulations such as gel formulations may include an organic solvent (e.g., a lower alkyl alcohol, such as ethyl alcohol or isopropyl alcohol; a ketone, such as acetone or N-methyl pyrrolidone; a glycol, such as propylene glycol; and the like, or mixtures thereof) present in an amount of 1% to 99%. In particular implementations, an organic solvent may be present in an amount of 60% to 80%. In various implementations, topical formulations may include a cellulose derivative, such as hydroxyl ethyl cellulose, hydroxy propyl cellulose, hydroxy propyl methyl cellulose, methyl cellulose, carboxy methyl cellulose, sodium carboxy methyl cellulose, ethyl cellulose, and the like, or combinations thereof present in an amount of 0.1% to 20%. In particular implementations, a cellulose derivative may be present in an amount of 0.5% to 5%.

In various implementations, topical formulations such as gel formulations may include any suitable tonicity modifier. Exemplary suitable tonicity modifiers include sodium chloride, potassium chloride, mannitol, sucrose, lactose, fructose, maltose, dextrose, dextrose anhydrous, propylene glycol, and glycerol. In various implementations, the tonicity modifier can be present in an amount of 0.5% to 1% by weight. In particular implementations, a tonicity modifier can be present in an amount of 0.8% to 1% by weight of the topical formulation. In various implementations, buffers can be present in the topical formulations. Exemplary buffers include phosphate buffered saline (PBS) acetate buffers, such as sodium acetate trihydrate or glacial acetic acid; and citrate buffers, such as sodium citrate dihydrate and citric acid.

In some implementations, topical formulations such as gel formulations may have a viscosity of at least 1,000 centipoise (cps). In other implementations, topical formulations such as gel formulations may have a viscosity of at least 3,000 cps. In specific implementations, the viscosity of topical formulations will not exceed 50,000 cps.

Powders and sprays particularly may benefit from the inclusion of excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane. The compositions of the disclosure can be alternatively administered by aerosol. This is accomplished by preparing an aqueous aerosol, liposomal preparation, or solid particles containing a composition of the disclosure. A non-aqueous (e.g., fluorocarbon propellant) suspension also could be used. Sonic nebulizers can be preferred because they minimize exposing the compositions to shear, which can result in degradation of the composition.

Although FIG. 1 illustrates the topical formulation 110 of the inhibitor 102, implementations of the present disclosure are not so limited. In various implementations, the inhibitor 102 can be formulated for intravenous injection, intradermal injection, intramuscular injection, oral administration, subcutaneous administration, intralesional administration, or topical administration.

In some implementations, the inhibitor 102 is administered with a pharmaceutically acceptable carrier, also referred to as an “excipient.” Any pharmaceutically acceptable carrier known in the art may be used to prepare a topical formulation. Exemplary pharmaceutically acceptable carriers and formulations are disclosed in Remington's Pharmaceutical Sciences, 18^{th} Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, solubilizing agents, skin protectants, moisturizers, emollients, chelating agents (e.g., EDTA), gels, binders, disintegration agents, lubricants, propellants, penetration enhancing agents, and/or additional therapeutic agents used in combination to the inhibitor 102.

Exemplary antioxidants include, but are not limited to, ascorbic acid and its esters, sodium bisulfite, butylated hydroxytoluene, butylated hydroxyanisole, tocopherols, and chelating agents like EDTA and citric acid.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol, or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers, or polysaccharides.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

Exemplary solubilizing agents include, but are not limited to, quaternary ammonium chloride, cyclodextrins, benzyl benzoate, lecithin, and polysorbates.

Exemplary skin protectants that can be used in a topical formulation include, but are not limited to, vitamin E oil, allantoin, dimethicone, glycerin, petrolatum, and zinc oxide. Exemplary moisturizers include, but are not limited to, glycerin, sorbitol, polyethylene glycols, urea, and propylene glycol.

In certain implementations, a topical formulation may include a penetration enhancing agent. The choice of topical formulation will depend on several factors, including the condition to be treated, the physicochemical characteristics of the therapeutic agent (e.g., the inhibitor 102) and other excipients present, their stability in the formulation, available manufacturing equipment, and costs constraints. As used herein, the term “penetration enhancing agent” refers to an agent capable of transporting a pharmacologically active compound through the stratum corneum and into the epidermis or dermis, preferably with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press. Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, III. (1997). In certain implementations, penetration agents include, but are not limited to, triglycerides (e.g., soybean oil), aloe compositions (e.g., aloe-vera gel), ethyl alcohol, isopropyl alcohol, octylphenol polyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methyl pyrrolidone.

It will also be appreciated that the compounds and pharmaceutical compositions disclosed herein can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a compound disclosed herein may be administered concurrently with another inhibitory agent), or they may achieve different effects (e.g., control of any adverse effects).

In certain implementations, the pharmaceutical compositions disclosed herein include one or more additional therapeutically active ingredients (e.g., anti-inflammatory and/or palliative). As used herein, the term “palliative” refers to treatment that is focused on the relief of symptoms of a disease and/or side effects of a therapeutic regimen, but is not curative. For example, palliative treatment encompasses pain killers, steroids, antimicrobials, antioxidants, retinoids, or antifungals.

Compositions can also be incorporated into wound dressings (e.g., bandages, adhesive bandages, transdermal patches). Generally, in these implementations, compositions are embedded within puffs, gauzes, fleeces, gels, powders, sponges, or other materials that are associated with a second layer to form a wound dressing. Absorption enhancers can also be used to increase the flux of the composition, and particularly the therapeutic agent within the composition, across the skin. The rate of such flux can be controlled by either providing a rate controlling membrane or dispersing the therapeutic agent in a polymer matrix or gel.

In particular implementations, the second layer of a wound dressing can be an elastomeric layer, vapor-permeable film, waterproof film, a woven or nonwoven fabric, mesh, or the like. The composition containing layer and second layer can be bonded using any suitable method (e.g., the application of adhesives, such as pressure sensitive adhesives, hot melt adhesives, curable adhesives; the application of heat or pressure, such as in lamination; a physical attachment through the use of stitching, studs, other fasteners; or the like).

Wound dressings may include adhesives for attachment to the skin or other tissue. Although any adhesive suitable for forming a bond with the skin or other tissue can be used, in certain implementations a pressure sensitive adhesive is used. Pressure sensitive adhesives are generally defined as adhesives that adhere to a substrate when a light pressure is applied but leave little to no residue when removed. Pressure sensitive adhesives include solvent in solution adhesives, hot melt adhesives, aqueous emulsion adhesives, calenderable adhesives, and radiation curable adhesives.

Some commonly used elastomers in pressure sensitive adhesives can include natural rubbers, styrene-butadiene latexes, polyisobutylene, butyl rubbers, acrylics, and silicones. In particular implementations, acrylic polymer or silicone-based pressure sensitive adhesives can be used. Acrylic polymers can often have a low level of allergenicity, be cleanly removable from skin, possess a low odor, and exhibit low rates of mechanical and chemical irritation. Medical grade silicone pressure sensitive adhesives can be chosen for their biocompatibility.

Amongst the factors that influence the suitability of a pressure sensitive adhesive for use in wound dressings of particular implementations is the absence of skin irritating components, sufficient cohesive strength such that the adhesive can be cleanly removed from the skin, ability to accommodate skin movement without excessive mechanical skin irritation, and good resistance to body fluids.

In particular implementations, the pressure sensitive adhesive can include a butyl acrylate. While butyl acrylate pressure sensitive adhesives can generally be used for many applications, any pressure sensitive adhesive suitable for bonding skin can be used. Such pressure sensitive adhesives are well known in the art.

In various implementations, the inhibitor 102 may be administered to the subject 104 for treatment of the skin fragility disease. Treating subjects includes delivering therapeutically effective amounts. Therapeutically effective amounts include those that provide prophylactic treatments and/or therapeutic treatments.

An “effective amount” is the amount of a formulation used to result in a desired physiological change in the subject 104. For example, an effective amount can provide a change in a metric associated with the skin fragility disease (e.g., a severity or frequency of symptoms or a change in a marker associated with the skin fragility disease). In various implementations, the marker may be SERCA2, ATP2A2, ATP2C1, SPCA1, KRT1, KRT2, KRT5, KRT10, KRT14, KRT16, KRT17, DSG1, DSG2, DSG3, DSG4, DSC1, DSC2, DSC3, plakoglobin, desmoplakin, plakophilin, ERK, pERK, B-RAF, C-RAF, or MEK. Effective amounts are often administered for research purposes. Effective amounts disclosed herein can cause a statistically significant effect in an animal model or in vitro assay relevant to the assessment of a pathology's development or progression.

A “prophylactic treatment” includes a treatment administered to a subject (e.g., the subject 104) who does not display signs or symptoms of the skin fragility disease or displays only early signs or symptoms of the skin fragility disease such that treatment is administered for the purpose of diminishing or decreasing the risk of developing the skin fragility disease further. For example, a subject may be genetically predisposed to the skin fragility disease. In some examples, a subject may be administered a BRAF inhibitor, and therefore may be more likely to develop a skin fragility disease. Examples of BRAF inhibitors include dabrafenib, vemurafenib, sorafenib, and encorafenib. Thus, a prophylactic treatment functions as a preventative treatment against the skin fragility disease. In particular implementations, prophylactic treatments reduce or delay physical symptoms associated with the skin fragility disease.

A “therapeutic treatment” includes a treatment administered to a subject (e.g., the subject 104) who displays symptoms or signs of the skin fragility disease and is administered to the subject for the purpose of diminishing or eliminating those signs or symptoms of the skin fragility disease. The therapeutic treatment can reduce, control, or eliminate the presence or activity of the skin fragility disease and/or reduce control or eliminate side effects of the skin fragility disease.

Function as an effective amount, prophylactic treatment or therapeutic treatment are not mutually exclusive, and in particular implementations, administered dosages may accomplish more than one treatment type.

In particular implementations, therapeutically effective amounts induce a reduced progression of a pathology associated with SERCA2, ATP2A2, ATP2C1, SPCA1, KRT1, KRT2, KRT5, KRT10, KRT14, KRT16, KRT17, DSG1, DSG2, DSG3, DSG4, DSC1, DSC2, DSC3, plakoglobin, desmoplakin, plakophilin, ERK, pERK, B-RAF, C-RAF, or MEK. In particular implementations, the reduced progression includes reduced or stabilized symptoms, reduced or stabilized levels of one or more markers associated with the pathology, or a change in another metric known in the art.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject (e.g., the subject 104) can be determined by a physician, veterinarian, or researcher taking into account parameters such as physical and physiological factors including target, body weight, severity of condition, type of condition, stage of condition, previous or concurrent therapeutic interventions, idiopathy of the subject, and route of administration.

Useful doses, in some examples, are reported as a mass of the active ingredient (e.g., the inhibitor 102) per weight of the subject. Useful doses can range from 0.1 μg/kg to 2 mg/kg. In other examples, a dose can include 0.1 μg/kg, 0.2 μg/kg, 0.5 μg/kg, 1 μg/kg, 10 μg/kg, 20 μg/kg, 50 μg/kg, 100 μg/kg, 250 μg/kg, 500 μg/kg, 750 μg/kg, 1000 μg/kg, 0.1 to 5 mg/kg or from 0.5 to 1 mg/kg. In other examples, a dose can include 1 mg/kg, 1.25 mg/kg, 1.5 mg/kg, 1.75 mg/kg, 2 mg/kg, 5 mg/kg, or more.

In some implementations, useful doses are reported as the mass percentage of an active ingredient. Useful doses of a topical treatment can range from 0.001% to 10% or from 0.05% to 2.5%. In some examples, a dose can include 0.001%, 0.002%, 0.003%, 0.005%, 0.01%, 0.02%, 0.03%, 0.05%, 0.075%, 0.1%, 0.2%, 0.3%, 0.5%, 1%, 2%, 3%, 5%, 7.5%, or 10%.

Therapeutically effective amounts can be achieved by administering single or multiple doses during the course of a treatment regimen (e.g., five times a day, four times a day, three times a day, twice a day, daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months, or yearly). In particular implementations, the treatment protocol may be dictated by a clinical trial protocol or a US FDA-approved treatment protocol.

### First Experimental Example

### Introduction

Mutation of the ATP2A2 gene encoding sarco-endoplasmic reticulum calcium ATPase 2 (SERCA2) was linked to Darier disease (DD) more than two decades ago; however, there remain no targeted therapies for this disorder causing recurrent skin blistering and infections. Since Atp2a2 knockout mice do not phenocopy its pathology, a human tissue model of DD was developed to elucidate its pathogenesis and identify potential therapies and is described in this experimental example. Cultured human keratinocytes and organotypic epidermis (Simpson C L, et al. Methods Mol Biol. 2010; 585: p. 127-46) were leveraged to establish a pre-clinical model of DD to better elucidate its pathogenesis and identify new therapeutic avenues to help direct future clinical trials. Using CRISPR/Cas9 gene editing (Guitart J R, Jr., et al. J Invest Dermatol. 2016; 136(9): p. e87-e93; Sarkar M K, et al. Ann Rheum Dis. 2018; 77(11): p. 1653-1664), human keratinocytes having the first exon of ATP2A2 disrupted were generated in this experimental example. The generated keratinocytes lacked SERCA2, which replicated features of DD, including weakened intercellular adhesion and defective differentiation in organotypic epidermis. To identify pathogenic drivers downstream of SERCA2 depletion, RNA sequencing and proteomic analysis were performed. SERCA2-deficient keratinocytes lacked desmosomal and cytoskeletal proteins required for epidermal integrity and exhibited excess mitogen-activated protein kinase (MAPK) signaling, which modulates keratinocyte adhesion and differentiation. Immunostaining patient biopsies substantiated these findings with lesions showing keratin deficiency, cadherin mis-localization, and extracellular signal-regulated kinase (ERK) hyper-phosphorylation. Dampening ERK activity with mitogen-activated protein kinase kinase (MEK, also referred to as MAPKK) inhibitors rescued adhesive protein expression and restored keratinocyte sheet integrity despite SERCA2 depletion or chemical inhibition. The multi-omic analysis revealed inhibition of MEK or the MAPK/ERK pathway as a novel treatment strategy for DD. This experimental example describes coupling multi-omic analysis with human organotypic epidermis as a pre-clinical model, which revealed that SERCA2 haploinsufficiency disrupts critical adhesive components in keratinocytes via extracellular signal-regulated kinase (ERK) signaling and identified MEK inhibition as a treatment strategy for DD.

### Methods and Materials

### Reagents

The SERCA2 inhibitor thapsigargin (Cat. #12758), MEK inhibitors trametinib (Cat. #62206), U0126 (Cat. #9903), PD98059 (Cat. #9900), and PD184352 (Cat. #12147), and B-Raf inhibitor dabrafenib (Cat. #91942) are from Cell Signaling Technology of Danvers, MA. Rabbit antibodies against SERCA2 (D51B11; Cat. #9580), 23hosphor-ERK1/2 (D13.14.4E; Cat. #4370) and mouse antibodies again ERK1/2 (L34F12; Cat. #4696) and β-Actin (8H10D10; Cat. #3700) are from Cell Signaling Technology. Rabbit anti-Cytokeratin 10 (Cat. #ab76318), mouse anti-Cytokeratin 14 (Cat. #ab7800), mouse anti-Desmoglein 1 (Cat. #ab12077), and mouse anti-Involucrin (Cat. #ab68) are from Abcam of Cambridge, United Kingdom. For fluorescent immunoblotting, IRDye 800CW goat anti-rabbit IgG (Cat. #926-32211) and IRDye 680RD goat anti-mouse IgG (Cat. #926-68070) are from LI-COR Biosciences of Lincoln, NE. For fluorescent immunostaining, secondary antibodies are from Thermo-Fisher of Waltham, MA: Goat anti-mouse IgG with Alexa Fluor 405 (Cat. #A31553), Alexa Fluor 488 (Cat. #A11001), Alexa Fluor 594 (Cat. #A11005), or Alexa Fluor 633 (Cat. #A21050); goat anti-rabbit IgG with Alexa Fluor 405 (Cat. #A31556), Alexa Fluor 488 (Cat. #A11008), Alexa Fluor 594 (Cat. #A11012), or Alexa Fluor 633 (Cat. #A21070). Hoechst 33342 is from Thermo-Fisher (Cat. #H1399).

### Cell Culture

Human telomerase reverse transcriptase (hTERT)-immortalized human epidermal keratinocytes (THEKs) from original line N/TERT-2G (Dickson M A, et al. Mol Cell Biol. 2000; 20(4): p. 1436-47) (a gift from James Rheinwald, Brigham and Women's Hospital, Boston, Massachusetts, USA) were grown in keratinocyte serum-free medium (KSFM) ordered as a kit (Thermo-Fisher Cat. #37010022) with supplements of 0.2 ng/mL human epidermal growth factor and 30 μg/mL bovine pituitary extract. Other additives included 0.31 mM CaCl₂, 100 U/mL penicillin, and 100 μg/mL streptomycin.

Normal human epidermal keratinocytes (NHEKs) were procured as described below and grown in Medium 154 with 0.07 mM CaCl₂ (Thermo-Fisher Cat. #M154CF500) and 1× human keratinocyte growth supplement (Thermo-Fisher Cat. #S0015), and 1× gentamicin/amphotericin (Thermo-Fisher Cat. #R01510).

J2-3T3 immortalized murine fibroblasts (a gift from Kathleen Green, Northwestern University, Chicago, Illinois, USA) were grown in complete Dulbecco's Modified Eagle Medium (DMEM) (Thermo-Fisher Cat. #11965092) supplemented with 10% fetal bovine serum (FBS) (Hyclone, Fisher Scientific of Waltham, MA, Cat. #SH3039603), 2 mM GlutaMAX (Thermo-Fisher Cat. #35050061), 100 U/mL penicillin, and 100 μg/mL streptomycin.

All cell lines were maintained at 37° C. in 5% CO₂ in an air-jacketed, humidified incubator. Cells were grown on sterile cell culture dishes and passaged at sub-confluency using 0.25% Trypsin-ethylenediaminetetraacetic acid (EDTA) (Thermo-Fisher Cat. #15400054).

### Organotypic Epidermal Culture

Human organotypic epidermal “raft cultures” were generated as described (Simpson C L, et al. Methods Mol Biol. 2010; 585: p. 127-46; Simpson C L, et al. Cell Rep. 2021; 34(5): p. 108689). Cultures were differentiated using E-medium, a 3:1 mixture of DMEM:Ham's F12 (Thermo-Fisher Cat. #11765054) with 10% FBS, 180 μM adenine (Sigma of Burlington, MA, Cat. #A2786), 0.4 μg/mL hydrocortisone (Sigma, Cat. #H0888), 5 μg/mL human insulin (Sigma Cat. #91077C), 0.1 nM cholera toxin (Sigma, Cat. #C8052), 5 μg/mL apo-transferrin (Sigma Cat. #T1147), and 1.36 ng/mL 3,3′,5-tri-iodo-L-thyronine (Sigma Cat. #T6397).

J2-3T3 fibroblasts were seeded into collagen matrix rafts within Transwells (Corning of Corning, NY, Cat. #353091). For each raft, 1×10⁶ fibroblasts were resuspended in 1/10 the final volume of sterile filtered reconstitution buffer (1.1 g of NaHCO₃ plus 2.39 g of 4-(2-hydroxyethyl)piperazine-1-ethane-sulfonic acid (HEPES) in 50 mL 0.05N NaOH), and then 1/10 the final volume of 10×DMEM (Millipore Sigma of Burlington, MA, Cat. #D2429) was added. The cells were mixed thoroughly by pipetting, and then high-concentration rat tail collagen I (Corning, Cat. #CB354249) was added (4 mg/mL final concentration), along with sterile deionized H₂O to bring the solution up to the final volume. If necessary, 0.05N NaOH was added dropwise to adjust the pH to 7 based on the phenol red indicator. The collagen-fibroblast slurry was mixed via inversion, then pipetted into a Transwell insert placed within deep, 6-well cell culture plates (Corning, Cat. #08-774-183). The rafts were polymerized at 37° C. for 1 hour, after which they were submerged in complete DMEM and incubated at 37° C. overnight.

Next, confluent keratinocyte cultures were trypsinized and resuspended in E-medium with epidermal growth factor (EGF) (5 ng/ml) to final concentration of 0.5×10⁶ cells/mL (2 mL per organotypic culture). The DMEM was aspirated from the upper and lower transwell chambers, then 2 mL (1×10⁶ cells) of the keratinocyte suspension were pipetted on top each raft. E-medium with EGF was added to the bottom transwell chamber to submerge the raft, and the cultures were incubated at 37° C. After 24 hours, E-medium was aspirated from the top and bottom chambers. An air-liquid interface was established to induce stratification by adding E-medium (without EGF) only to the bottom chamber to reach the bottom of the raft. Organotypic cultures were grown at 37° C. for up to 12 days. E-medium in the bottom chamber was replaced every other day. For drug treatments, inhibitors or vehicle control (dimethyl sulfoxide (DMSO)) were diluted in E-medium in the bottom chamber. Inhibitors were used at the following concentrations: Thapsigargin (1 μM), trametinib (1 μM), and PD98059 (20 μM). For histologic examination, the transwell was moved to a standard 6-well cell culture plate and submerged in 10% neutral-buffered formalin (Fisher Scientific Cat. #22-026-435) for at least 24 hours. Organotypic cultures were processed for histologic examination by Core A of the Penn Skin Biology and Diseases Resource-based Center (SBDRC) of Philadelphia, PA or the Experimental Histopathology Core of the Fred Hutchinson Cancer Center of Seattle, WA.

### CRISPR/Cas9 Gene Editing

CRISPR knockout (KO) keratinocytes were generated as described (Sarkar M K, et al. Ann Rheum Dis. 2018; 77(11): p. 1653-1664). Single-guide RNAs (sgRNAs) were designed to target the ATP2A2 gene (sgRNA2: GTTTTGGCTTGGTTTGAAGA (SEQ ID NO: 1)) or the TUBAP pseudogene (sgRNA1: GTATTCCGTGGGTGAACGGG (SEQ ID NO: 2)) to generate a control KO line using a web tool for CRISPR design (https://portals.broadinstitute.org/gpp/public/analysis-tools/sgrna-design). Synthetic sgRNA target sequences were inserted into a cloning backbone, pSpCas9 (BB)-2A-GFP (PX458) (Addgene of Watertown, MA, Cat. #48138), and then cloned into competent E. coli (Thermo-Fisher, Cat. #C737303). Proper insertion was validated by Sanger sequencing. The final plasmid was transfected into an hTERT-immortalized human epidermal keratinocyte line (N/TERT-2G) (Dickson M A, et al. Mol Cell Biol. 2000; 20(4): p. 1436-47) using the TransfeX transfection kit (American Type Culture Collection (ATCC) of Manassas, VA, Cat. #ACS4005) in the presence or absence of JAK1/JAK2 inhibitor, baricitinib (10 μg/mL). Green fluorescent protein (GFP)-positive single cells were plated and expanded. Cells were genotyped and analyzed by Sanger sequencing to confirm the presence of heterozygous or homozygous mutations in the target gene.

### Determination of Cell Proliferation

THEKs were seeded at a density of 1.25×10⁵ cells per well of 6-well culture dishes in complete KSFM. Cells were then transferred to a CELLCYTE X live-cell imaging system (Cytena of Freiburg, Germany) to determine proliferation rates of label-free cells. Images of 25 nonoverlapping fields per well were acquired every 2 hours using the 10× objective in the enhanced contour channel. Images were analyzed using the CELLCYTE analysis software to measure cell confluence, and growth rates were extrapolated by simple linear regression using GraphPad Prism software (GraphPad of La Jolla, CA).

### RNA Sequencing (RNAseq)

RNAseq libraries were prepared for transcriptomics analysis as described (Egolf S, et al. Sci Adv. 2021; 7(50): p. eabj9141). THEKs were grown to confluency in 10 cm cell culture dishes in keratinocyte serum-free medium (KSFM), then differentiated in E-medium for 72 h. RNA was isolated from THEKs using the Rneasy kit (Qiagen of Hilden, Germany) according to the manufacturer's instructions. Messenger RNAs (mRNAs) were isolated using the NEBNext Poly(A) mRNA magnetic isolation module (New England Biolabs of Ipswich, MA). RNAseq libraries were prepared for Illumina using the NEBNext Ultra-Directional RNA library preparation kit. Library quality was confirmed using an Agilent BioAnalyzer 2100 (Agilent of Santa Clara, CA) and quantified using the NEBNext Library Quant Kit for Illumina. Sequencing was performed using the Illumina NextSeq500 platform (Illumina of San Diego, CA) employing a single-end, 75-base pair sequencing strategy. All RNA-seq reads were then aligned to the Homo sapiens reference genome (University of California, Santa Cruz (UCSC) hg19, RefSeq from the National Center for Biotechnology Information (NCBI) and Gencode gene annotations) using RNA Spliced Transcripts Alignment to a Reference (STAR) under default settings (Dobin A, et al. Bioinform. 2013; 29(1): p. 15-21). Fragments per kilobase per million mapped fragments (FKPM) generation and differential expression analysis were done using the DESeq2 package (Love M I, et al. Genome Biol. 2014; 15:550). Statistical significance was determined using an adjusted P value (P_{adj}) of ≤0.05.

### Gene Ontology Analyses

Gene ontology (GO) analyses of RNAseq data were performed as described (Egolf S, et al. Sci Adv. 2021; 7(50): p. eabj9141) using PANTHER at http://pantherdb.org/. Enrichment analysis under the category “biological process” was performed to identify statistically overrepresented GO terms. Statistical significance was determined using Fisher's exact test. Normalized enrichment scores and false discovery rates were computed for each biological process. GO terms were plotted using Prism 9 (GraphPad).

### Whole-Cell Proteomics

THEKs were grown to confluency in 10 cm cell culture dishes in complete KSFM, then differentiated in E-medium for 72 hours. Cells were then washed with phosphate-buffered saline (PBS) and lysed in ice-cold lysis buffer [0.1 M Ammonium Bicarbonate, 8 M Urea, 0.1% (w/v) RapiGest SF (Waters of Milford, MA)]. Lysates were homogenized using a microtip probe sonicator (Fisher Scientific) and clarified by centrifugation. Clarified supernatants were transferred to a new microcentrifuge tube and protein concentrations were determined by bicinchoninic acid (BCA) assay (Pierce of Appleton, WI). After normalizing sample volumes, 200 μg of protein from each sample were reduced by the addition of tris(2-carboxyethyl)phosphine (TCEP) to a final concentration of 5 mM for 1 hour at room temperature. Protein samples were then alkylated for 30 minutes at room temperature by adding iodoacetamide to a final concentration of 10 mM. Unreacted iodoacetamide was quenched by adding TCEP to a final concentration of 10 mM for 30 minutes at room temperature. Following reduction and alkylation, samples were diluted with 0.1 M ammonium bicarbonate to decrease the urea concentration to 1.5 M. Protein samples were then digested overnight at 37° C. with Trypsin Gold (Promega of Madison, WI) according to the manufacturer's instructions. On the following day, each sample was adjusted to pH 2 with 2 M hydrochloric acid (HCl) to hydrolyze the RapiGest surfactant. The precipitated RapiGest was then removed via centrifugation at 10,000 times gravity (×g) for 5 minutes. Acetonitrile and trifluoroacetic acid were added to the clarified supernatants to final concentrations of 5% (volume/volume (v/v)) and 0.1% (v/v), respectively. Trypsin-digested peptides were then desalted using MacroSpin C18 columns (Pierce), according to the manufacturer's instructions. Following elution, peptides were completely dried under vacuum and resuspended in 0.1% (v/v) formic acid to a final concentration of 0.5 μg/mL. Samples were transferred into autosampler vials and subjected to mass spectrometry analysis using an Orbitrap Eclipse mass spectrometer (Thermo Scientific) as described (Cesinger M R, et al. Infect Immun. 2022; 90(10): p. e0021122). Raw spectral data were processed in MaxQuant using the Homo sapiens reference protein library (Universal Protein Resource (UniProt)) and relative protein abundances were determined using the label-free quantification (LFQ) intensity method as described (Cesinger M R, et al. Infect Immun. 2022; 90(10): p. e0021122).

### RNA Isolation and Real-Time Quantitative PCR

THEKs were seeded at a density of 1×10⁶ cells per well of 6-well cell culture dishes in complete KSFM. Upon reaching confluence, KSFM was replaced with E-medium containing vehicle control (DMSO) or inhibitors at the following concentrations: Trametinib (1 μM), U0126 (10 μM), or PD184352 (10 μM). At the indicated time points, cell culture medium was removed and cells were washed once with PBS. RNA was extracted using the NucleoSpin RNA Plus Kit (Machery-Nagel of Duren, Germany) followed by reverse-transcription with the iScript cDNA synthesis kit (Bio-Rad of Hercules, CA) according to the manufacturer's instructions. Copy DNAs, also referred to as complementary DNAs (cDNAs) were diluted 5-fold and 2 μL of the resulting cDNAs were used for each 10 μL quantitative polymerase chain reaction (qPCR) assay. Real-time qPCR assays were carried out using the TaqMan Gene expression master mix (Applied Biosystems of Waltham, MA) according to the manufacturer's protocol. Pre-designed TaqMan probes from Thermo-Fisher were used for quantification of human Actb (Hs01060665_g1), Krt1 (Hs00196158_m1), Krt10 (Hs00166289_m1), Krt14 (Hs00265033_m1), Dsg1 (Hs00355084_m1), Alox12b (Hs00153961_m1), Alox15b (Hs00153988_m1), Ifi27 (Hs01086373_g1), Trpv3 (Hs00376854_m1), Trpv4 (Hs01099348_m1), or Notch1 (Hs01062014_m1). The housekeeping gene Actb was used as an endogenous control and mRNA fold changes were determined using the 2^{(-ΔΔCt)} method.

### Immunoblotting

THEKs were seeded at a density of 1×10⁶ cells per well of 6-well cell culture dishes in KSFM. Upon reaching confluence, KSFM was replaced with E-medium containing vehicle control (DMSO) or inhibitors at the following concentrations: Dabrafenib (1 μM), trametinib (1 μM), U0126 (10 μM), PD184352 (10 μM), or PD98059 (20 μM). After 72 hours, whole-cell lysates were generated by washing cells once in PBS followed by lysis in urea sample buffer [8 M Urea, 60 mM Tris, 1% sodium dodecyl sulfate (SDS), 10% glycerol, 5% β-mercaptoethanol, 0.0005% pyronin-Y, pH 6.8] for 10 min. Lysates were homogenized using a microtip probe sonicator (Fisher Scientific).

Whole-cell lysates were loaded onto Any kD Mini-PROTEAN TGX Precast Protein Gels (Bio-Rad) and separated by electrophoresis. Proteins were transferred onto nitrocellulose membranes (Bio-Rad) in ice-cold Towbin transfer buffer (25 mM Tris, 192 mM glycine, 20% (v/v) methanol) at 100 volts (V) for 90 minutes at 4° C. Membranes were blocked in Intercept tris-buffered saline (TBS) blocking buffer (LI-COR) for 45 min at room temperature. Membranes were probed overnight at 4° C. with the primary antibodies diluted at 1:1000 in Intercept TBS blocking buffer (LI-COR). Blots were washed at least three times in 1×TBS containing 0.1% (v/v) Tween-20 (TBS-T), then incubated for 1 hour at room temperature with IRDye 800CW goat anti-rabbit IgG and/or IRDye 680RD goat anti-mouse IgG (LI-COR) diluted 1:10,000 in Intercept TBS blocking buffer. Blots were washed at least three times in TBS-T and proteins were visualized using an Odyssey Fc Imaging System (LI-COR).

### Fluorescent Immunocytochemistry

Keratinocytes were grown to confluence in 35 mm, glass-bottom cell culture dishes (MatTek of Ashland, MA, #P35G-1.5-20-C). For staining keratins and desmosomal proteins, cells were fixed in ice-cold 100% methanol at −20° C. for 2 minutes, allowed to dry, then re-hydrated in PBS. For staining other proteins, cells were fixed in 4% paraformaldehyde for 10 minutes at 37° C. Fixed cells were then incubated with blocking buffer [0.5% (w/v) bovine serum albumin (BSA, Sigma), 10% (w/v) normal goat serum (NGS, Sigma) in PBS] for 30 minutes at 37° C. Cells were then washed with PBS. Primary antibodies were diluted in 0.5% (w/v) BSA in PBS and incubated on the cells overnight at 4° C. Primary antibody dilutions were as follows: rabbit anti-SERCA2 (1:200), rabbit anti-cytokeratin 10 (1:2,000), mouse anti-Desmoglein 1 (1:100), mouse anti-Desmoglein 2 (1:100), mouse anti-Desmoglein 3 (1:100), mouse anti-Plakoglobin (1:100), rabbit anti-PERK1/2 (1:400), and mouse anti-ERK1/2 (1:400). Cells were washed three times with PBS, then incubated with species-specific secondary antibodies diluted at 1:300 (with or without Hoechst at 1:500) in 0.5% (w/v) BSA in PBS for 30 min at 37° C. Cells were washed three times with PBS, then held in PBS for imaging using spinning-disk confocal microscopy, as detailed below.

### Histologic Analysis and Tissue Procurement

Paraffin-embedded formalin-fixed tissue cross-sections of organotypic epidermis or skin biopsies were processed for histology and stained with hematoxylin and eosin (H&E) using standard methods. H&E-stained glass slides were imaged on an EVOS FL imaging system (Thermo-Fisher) using an EVOS 40× long working distance, achromatic, phase-contrast objective (Thermo-Fisher). Images were captured using the embedded high-sensitivity interline CCD color camera.

### Fluorescent Immunohistochemistry

Paraffin-embedded formalin-fixed tissue cross-sections on glass slides were incubated at 65° C. for 2 h. Sections were prepared for staining by immersion in 3 baths of xylenes (Thermo-Fisher) for 5 min each, followed by 3 baths of 95% ethanol for 5 min each, then 70% ethanol for 5 minutes, and finally 3 baths of PBS for 5 min each. Slides were then submerged in antigen retrieval solution [0.1 M sodium citrate (pH 6.0) with 0.05% (v/v) Tween-20] and heated to 95° C. for 15 min. Slides were allowed to cool to room temperature and then washed with PBS. Tissue sections were encircled with a hydrophobic barrier using a PAP pen. Tissue sections were incubated in blocking buffer [0.5% (w/v) BSA, 10% (v/v) NGS in PBS] for 30 minutes at 37° C. in a humidified chamber. Slides were washed in 3 baths of PBS for 5 minutes each, then incubated with primary antibodies diluted in 0.5% (w/v) BSA in PBS overnight at 4° C. in a humidified chamber. Primary antibody dilutions were as follows: rabbit anti-cytokeratin 10 (1:3000), mouse anti-desmoglein 1 (1:50), rabbit anti-phospho-ERK (1:400), mouse anti-ERK (1:400). Slides were then washed in 3 baths of PBS for 5 minutes each and incubated with secondary antibodies diluted at 1:300 (with or without Hoechst at 1:500) in 0.5% (w/v) BSA in PBS for 60 minutes at 37° C. in a humidified chamber. Slides were washed in 3 baths of PBS for 5 minutes each and mounted in Prolong Gold (Invitrogen of Carlsbad, CA, Cat. #P36934) with a glass coverslip applied over the tissue sections. Slides were allowed to dry overnight prior to imaging by spinning-disk confocal microscopy, as detailed below.

### Fluorescence Microscopy Imaging

Images were acquired on a Hamamatsu ORCA-FusionBT sCMOS camera (Hamamatsu Photonics of Hamamatsu, Japan) using a Yokogawa W1 spinning-disk confocal (SDC) system (Yokogawa Electric of Musashino, Japan) on a Nikon Ti2 microscope (Nikon of Minato City, Japan). Samples were illuminated using 405, 488, 561, and 640 nm laser excitation lines and fluorescence was detected using a 60× 1.2 NA water objective (Nikon) with standard emission filters.

For live imaging of cytoplasmic calcium, THEKs were transduced with GcaMP6 (Addgene Cat. #40753) sub-cloned into the pLZRS retroviral vector. HEK293 Phoenix cells were grown in complete DMEM, then transfected with 4 μg pLZRS-GCaMP6 DNA plus 12 μl FuGENE 6 (Promega Cat. #E2691) in 800 μl of Opti-MEM (Thermo-Fisher Cat. #31985070), which was added to the cells and left overnight. Retroviral supernatants were collected the next day and polybrene (Sigma Cat. #H9268) was added at a concentration of 4 μg/ml. KSFM was removed from THEKs and replaced with viral medium for 1 h at 37° C. Cells were then washed in PBS and placed back in their normal medium and expanded in culture.

GCaMP6-transduced cells were seeded into 35 mm glass-bottom dishes in low-calcium medium (0.31 mM) and grown to confluency, then were imaged by confocal microscopy at 1 frame every 5 seconds. Cells were exposed to high-calcium (1.3 mM) after 60 seconds, then were imaged for an additional 5 minutes at 1 frame every 5 seconds. The Fiji “Measure” function (Fiji software, https://github.com/fiji/fiji) was used to calculate the mean intensity of GCaMP6 signal across the entire high-powered field (HPF) of the time series of images. The intensity at 0 seconds was subtracted from the intensity at each subsequent time point to calculate the change from baseline as a function of time across independent replicates for each genotype.

### Fluorescent Microscopy Quantification

Fluorescence microscopy images were analyzed using Fiji software. Quantification of fluorescence intensity was performed in a blinded manner using non-visibly identifiable microscopy images. The Fiji “Measure” function was used to calculate the mean intensity across the entire HPF of fixed cells or a region of interest (ROI) encompassing the entire epithelium circumscribed using the polygon selection tool. The mean intensity for each condition was averaged across multiple independent HPF for each biological replicate.

### Tissue Morphologic Quantification

Counts of retained nuclei in the cornified layers were performed by hand using non-visibly labeled H&E images to identify and count the numbers of retained nuclei per non-overlapping HPF for each condition, which were averaged across multiple independent experiments.

### Fluorescent Tissue Staining Quantification

Fluorescence images of immunostained tissue sections were captured by SDC microscopy as above and analyzed using Fiji. Quantification of fluorescence intensity was performed in using non-visibly labeled images from immunostained tissue cross-sections. The Fiji “Measure” function was used to calculate the mean intensity of the fluorescence signal and the mean background fluorescence intensity, which was subtracted from each value. The net fluorescence intensity was averaged across multiple non-overlapping HPF and the values from control cultures were normalized to an average value of 1.

### Monolayer Mechanical Dissociation Assay

Dispase-based mechanical dissociation assays were carried out as described (Simpson C L, et al. Am J Pathol. 2010; 177(6): p. 2921-37). Keratinocytes were plated at a density of 1×10⁶ cells per well of 6-well cell culture dishes. Upon reaching confluence, the calcium concentration of the medium was adjusted to 1.3 mM. Vehicle control (DMSO) or chemical inhibitors were added at the following concentrations: Thapsigargin (1 μM), dabrafenib (1 μM), trametinib (1 μM), U0126 (10 μM), PD184352 (10 μM), or PD98059 (20 μM). After 24 to 72 hours, monolayers were washed with PBS and then incubated with 500 μL dispase (5 U/ml) in Hank's balanced salt solution (Stemcell Technologies of Vancouver, CA, Cat. #07913) for 30 minutes at 37° C. Next, 4.5 ml PBS was added to the wells and all liquid plus released monolayers were transferred into 15 mL conical tubes, which were placed together in a rack and inverted 5-10 times to induce mechanical stress. Monolayer fragments were transferred back into 6-well cell culture plates and imaged with a 12-megapixel digital camera. Fragments were counted in Fiji.

### Statistical Analysis

Statistical analyses were performed using the open-source statistics package R or Prism version 9 (GraphPad), which was used to generate graphs. Statistical parameters including sample size, definition of center, dispersion measures, and statistical tests are included in each figure legend. Datasets were tested for normality using the D'Agostino-Pearson test. The means of two normally distributed groups were compared using a two-tailed unpaired Student's t test. The means of more than two normally distributed groups were compared using a one-way ordinary ANOVA followed by P-value adjustment for multiple comparisons. P-values less than 0.05 were considered statistically significant. Exact P-values are included in each figure.

### Study Approval

NHEKs from deidentified neonatal foreskins were procured by the Penn SBDRC under a protocol (808224) approved by the University of Pennsylvania Institutional Review Board (IRB). Tissue cross sections of deidentified skin biopsies were obtained from a tissue bank by the Penn SBDRC under a protocol (808225) approved by the University of Pennsylvania IRB. The use of deidentified tissues collected for clinical purposes that would otherwise be discarded was deemed exempt by the IRB from written informed consent.

### Data Availability

Values from all graphs are available in the Supporting Data Values file. Raw RNA-Seq data and raw proteomics data are also available in the Supporting Data Values file.

### Results

### Loss of SERCA2 in Human Keratinocytes Impairs Cytosolic Calcium Handling and Reduces Intercellular Adhesive Strength.

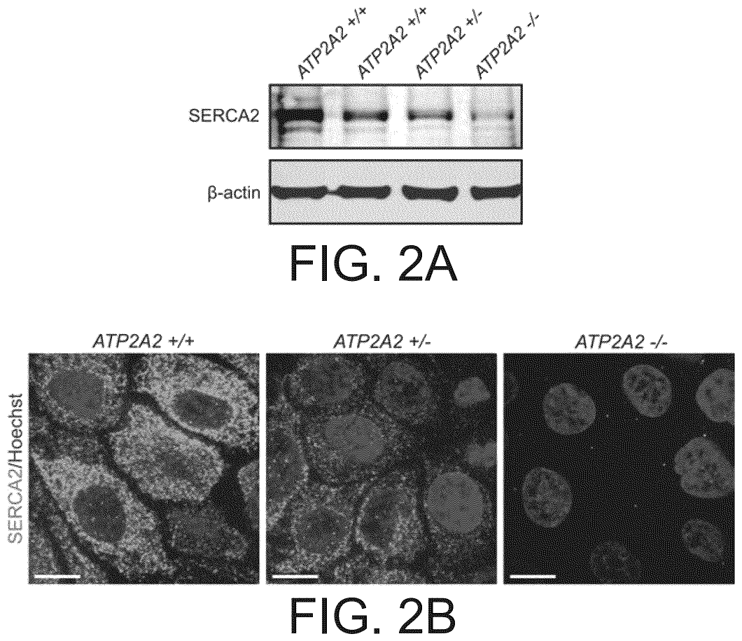

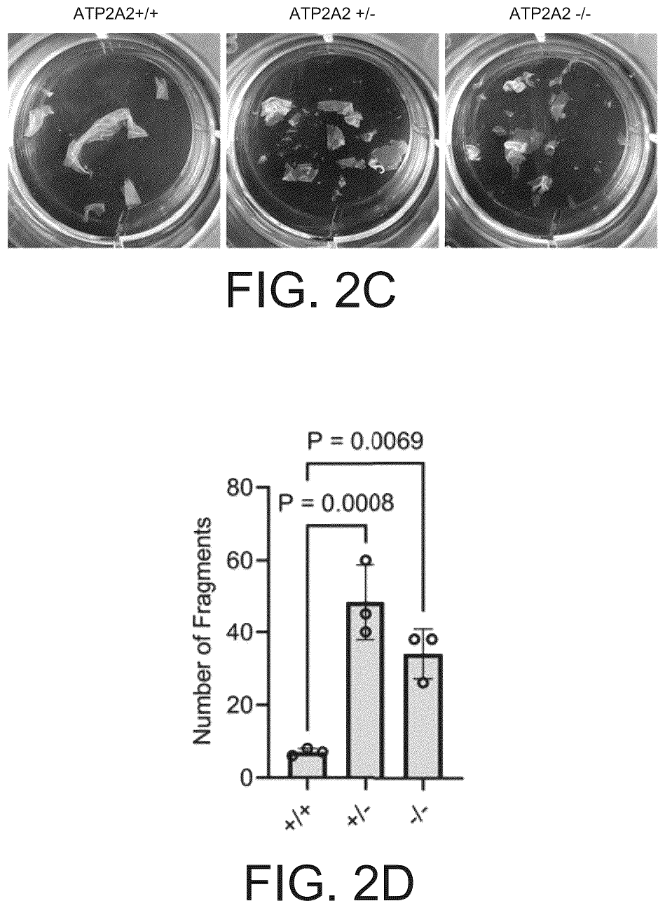

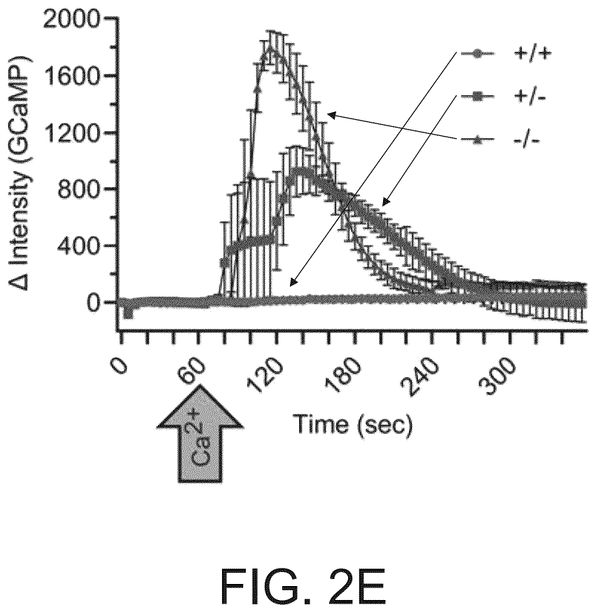

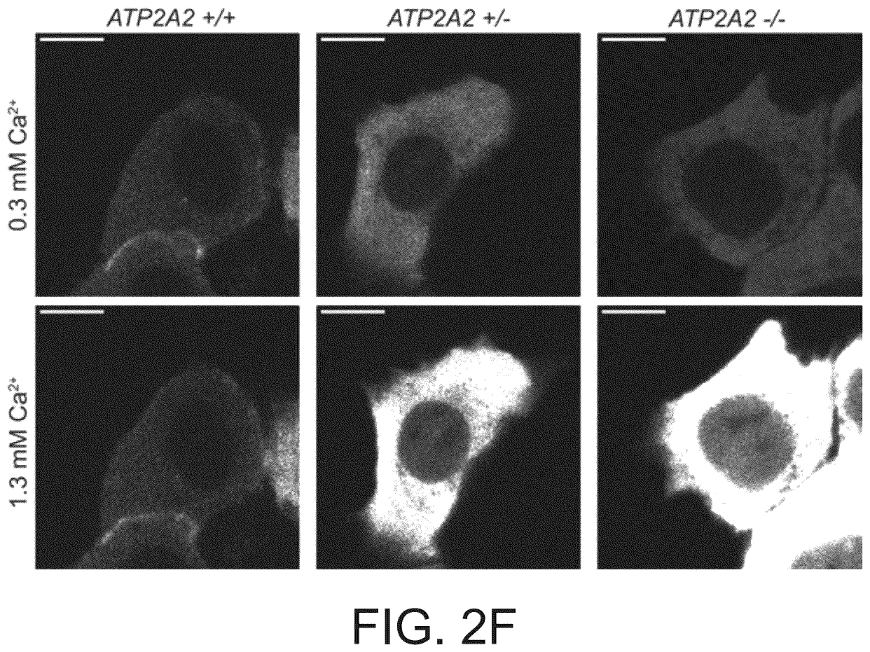

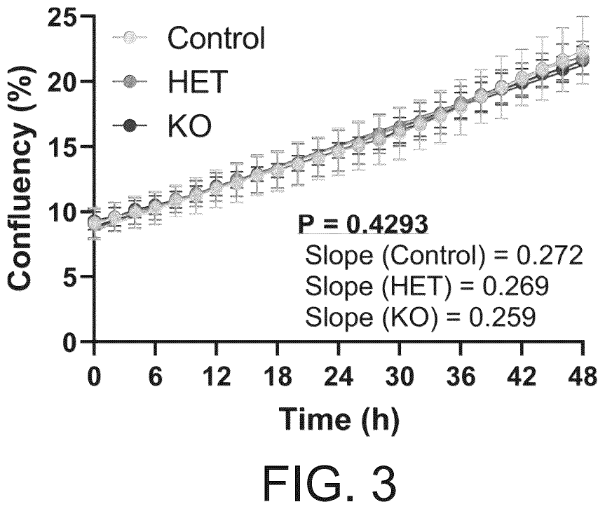

Since Atp2a2 KO mice did not phenocopy DD (Prasad V, et al. Cancer Res. 2005; 65(19): p. 8655-61), pre-clinical studies of its pathogenesis have been limited and no therapies have advanced to clinical trials to achieve FDA approval (Hanna N, et al. J Cutan Med Surg. 2022; 26(3): p. 280-290). Given the cutaneous pathology of DD is limited to the epidermis, CRISPR/Cas9 was used to ablate the ATP2A2 gene in keratinocytes (Sarkar M K, et al. Ann Rheum Dis. 2018; 77(11): p. 1653-1664), which can be grown into a fully stratified epidermis within an organotypic model (Simpson C L, et al. Methods Mol Biol. 2010; 585: p. 127-46). The first exon of ATP2A2 (or a pseudogene as a control) was targeted in hTERT-immortalized human epidermal keratinocytes (THEKs) from the parental line N/TERT-2G (Dickson M A, et al. Mol Cell Biol. 2000; 20(4): p. 1436-47); this allowed isolation of stable homozygous knockout (KO) and heterozygous (HET) cell lines harboring frameshift mutations that depleted SERCA2 as confirmed by immunoblotting cell lysates and immunostaining fixed cells (FIGS. 2A, 2B). SERCA2-deficient cells displayed similar proliferation rates to control cells (FIG. 3; supplemental material available online with this article; https://doi.org/10.1172/jci.insight.170739DS1).

Given the essential role of SERCA2 in assembling cell-cell junctions (Hobbs R P, et al. FASEB J. 2011; 25(3): p. 990-1001; Savignac M, et al. J Invest Dermatol. 2014; 134(7): p. 1961-1970), the mechanical integrity of control and SERCA2-deficient THEKs was assessed using an established dispase-based assay to quantify intercellular adhesive strength of epithelial cell monolayers (Hudson T Y, et al. Methods Cell Biol. 2004; 78: p. 757-86; Huen A C, et al. J Cell Biol. 2002; 159(6): p. 1005-17). Both HET and KO THEKs were found to exhibit reduced integrity, reflected by increased fragmentation of epithelial sheets upon mechanical stress (FIGS. 2C, 2D). GCaMP, a ratiometric green fluorescent protein (GFP)-based genetically encoded calcium indicator (Greenwald E C, et al. Chem Rev. 2018; 118(24): p. 11707-11794; Zaver S A, et al. J Invest Dermatol. 2023; 143(3): p. 353-361.E4), was used to confirm that depletion of the endoplasmic reticulum (ER)-localized calcium ATPase altered cytosolic calcium handling. THEKs were transduced with GCaMP, then imaged by live microscopy during exposure to increased extracellular calcium. While control cells demonstrated minimal change in GCaMP intensity after treatment with calcium, KO cells experienced large increases in cytosolic calcium; HET cells exhibited an intermediate phenotype, as would be expected with haploinsufficiency of SERCA2-driven transport of calcium into the ER (FIGS. 2E, 2F).

### Keratinocytes Lacking SERCA2 Exhibit Transcriptomic Alterations in Mediators of Epidermal Adhesion and Differentiation.

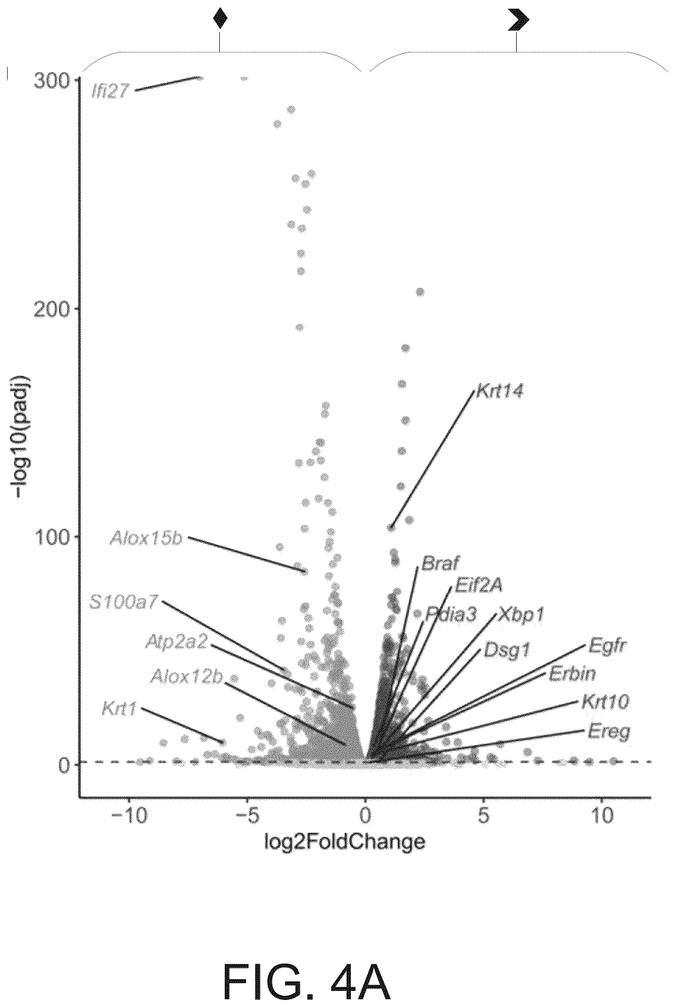

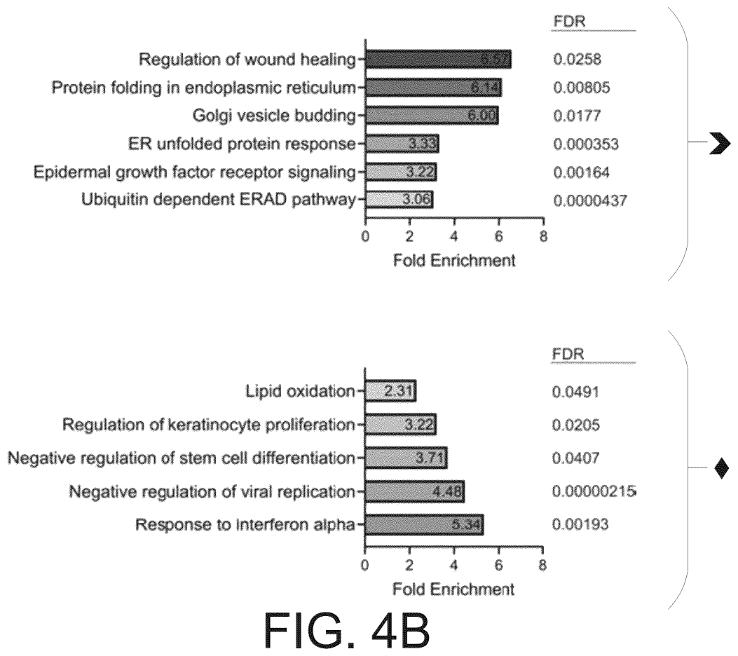

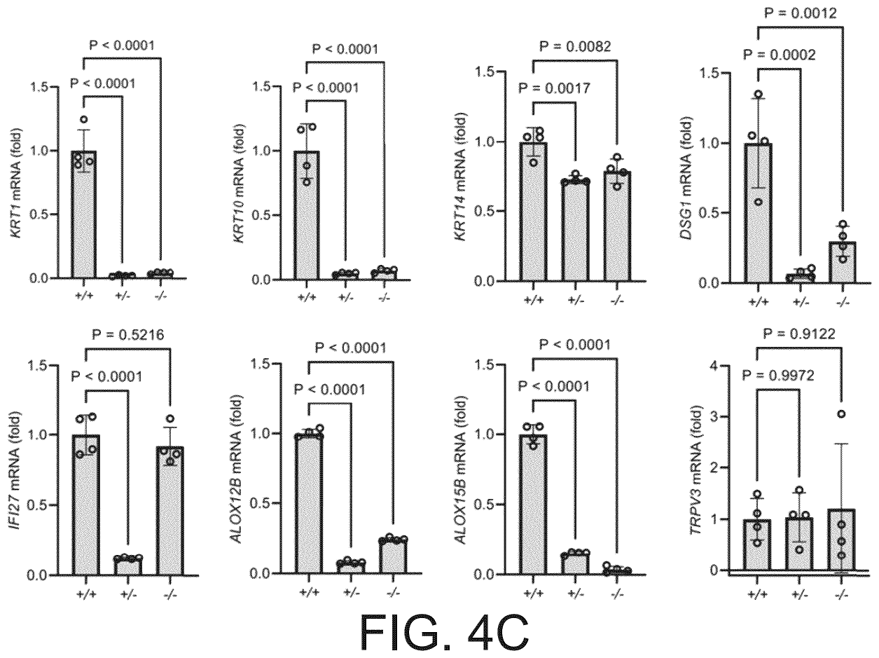

To identify potential pathogenic drivers of DD in an unbiased manner, downstream consequences of SERCA2 depletion on the transcriptome was assessed in HET and KO keratinocytes. Bulk RNA sequencing revealed that HET cells exhibited significant changes in mRNA transcript profiles with gene ontology (GO) analysis identifying alterations in gene subsets linked to intercellular adhesion, calcium regulation, and epidermal differentiation (FIGS. 4A, 4B), consistent with known pathologic features of DD. A gene signature indicating elevation in epidermal growth factor receptor (EGFR) signaling and the downstream mitogen-activated protein kinase (MAPK) pathway was also detected, both of which are known to regulate epidermal differentiation and adhesion (Hiratsuka T, et al. Proc Natl Acad Sci USA. 2020; 117(30): p. 17796-17807; Harmon R M, et al. J Clin Invest. 2013; 123(4): p. 1556-70; Kam C Y, et al. J Cell Biol. 2018; 217(9): p. 3219-3235; Egu D T, et al. Front Immunol. 2019; 10: p. 2883; Khavari T A, Rinn J. Cell Cycle. 2007; 6(23): p. 2928-31). These SERCA2 deficiency-related changes in gene expression were verified with quantitative reverse transcription PCR (RT-PCR) (FIG. 4C).

Relative to controls, HET keratinocytes exhibited marked reduction in mRNA encoding keratin 1 (KRT1) and keratin 10 (KRT10) intermediate filament cytoskeletal proteins and the desmosomal cadherin desmoglein 1 (DSG1), all of which are known to exhibit differentiation-dependent expression in the suprabasal epidermal layers (Simpson C L, et al. Nat Rev Mol Cell Biol. 2011; 12(9): p. 565-80). In contrast, the effect of SERCA2 deficiency on mRNA encoding the primary keratin of the basal epidermal layer, KRT14, was much smaller. While GO analysis identified changes in some differentiation-associated calcium-binding proteins, the mRNA encoding transient receptor potential vanilloid-3 (TRPV3), a plasma membrane calcium channel recently linked to calcium influx during the final stage of keratinocyte differentiation (Matsui T, et al. Proc Natl Acad Sci USA. 2021; 118(17)), was unchanged in HET or KO cells. In this example, some genes exhibited disparate effects in KO versus HET lines, such as the interferon response gene IFI27, which was only depleted in HET cells.

The expression of lipoxygenase genes that drive membrane lipid modifications is essential for terminal keratinocyte differentiation and epidermal barrier formation (Munoz-Garcia A, et al. Biochim Biophys Acta. 2014; 1841(3): p. 401-8; Crumrine D, et al. J Invest Dermatol. 2019; 139(4): p. 760-768) were also significantly reduced in SERCA2-deficient cells. Loss of ALOX12B, in particular, has been associated with autosomal recessive congenital ichthyosis (ARCI) (Jobard F, et al. Hum Mol Genet. 2002; 11(1): p. 107-13), a group of inherited dermatologic diseases characterized by skin scaling and epidermal barrier defects due to defective keratinocyte maturation (Crumrine D, et al. J Invest Dermatol. 2019; 139(4): p. 760-768; Sun Q, et al. JAMA Dermatol. 2022; 158(1): p. 16-25), which shares clinical features with DD and also responds to retinoid therapy (Zaenglein A L, et al. Pediatr Dermatol. 2021; 38(1): p. 164-180).

### Proteomic Analysis of SERCA2-Deficient Keratinocytes Reveals Alterations in Key Regulators of Tissue Integrity and Skin Barrier Formation.

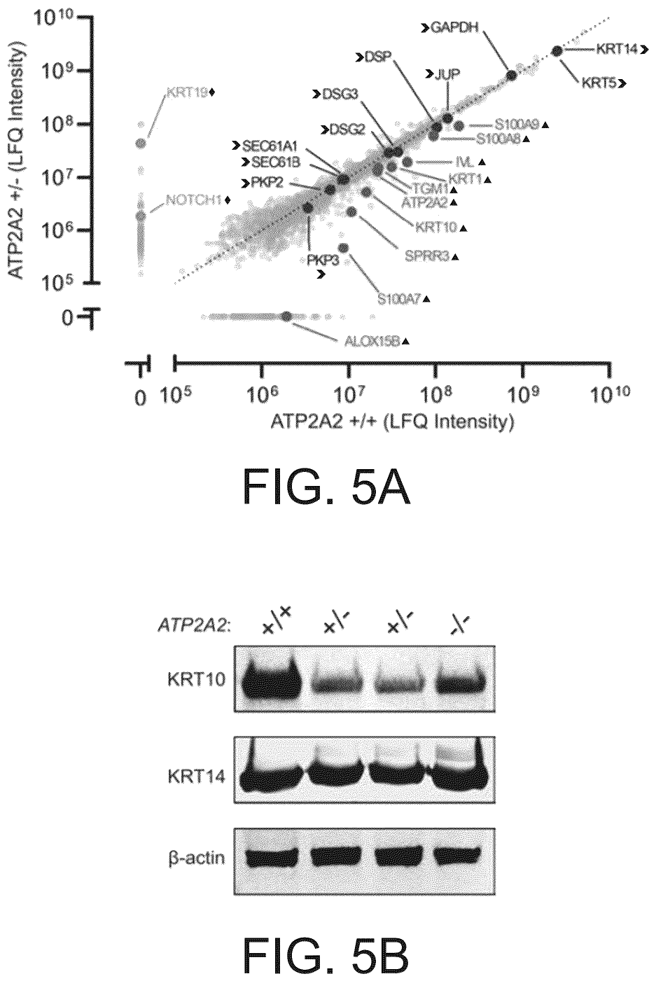

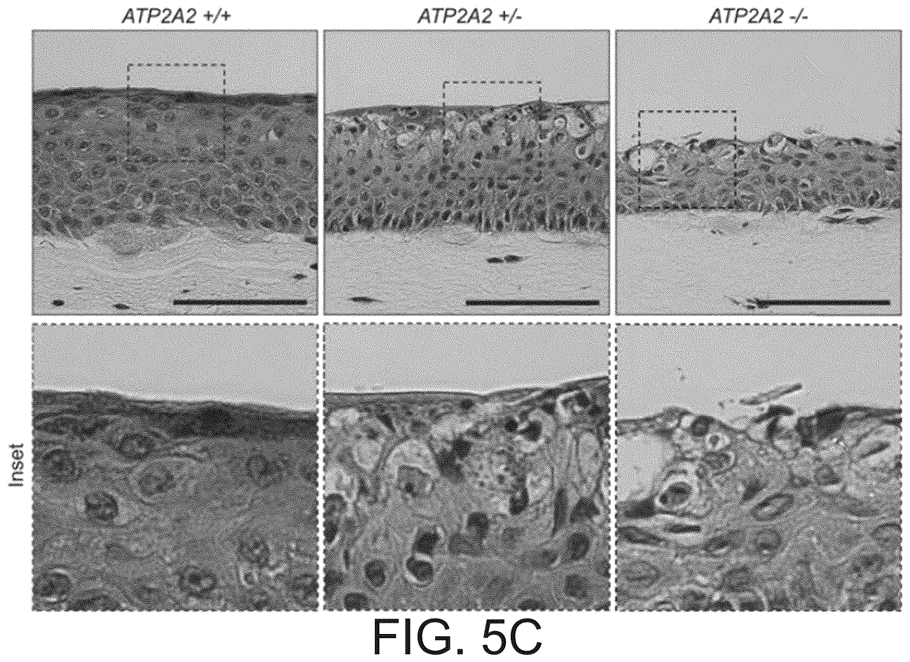

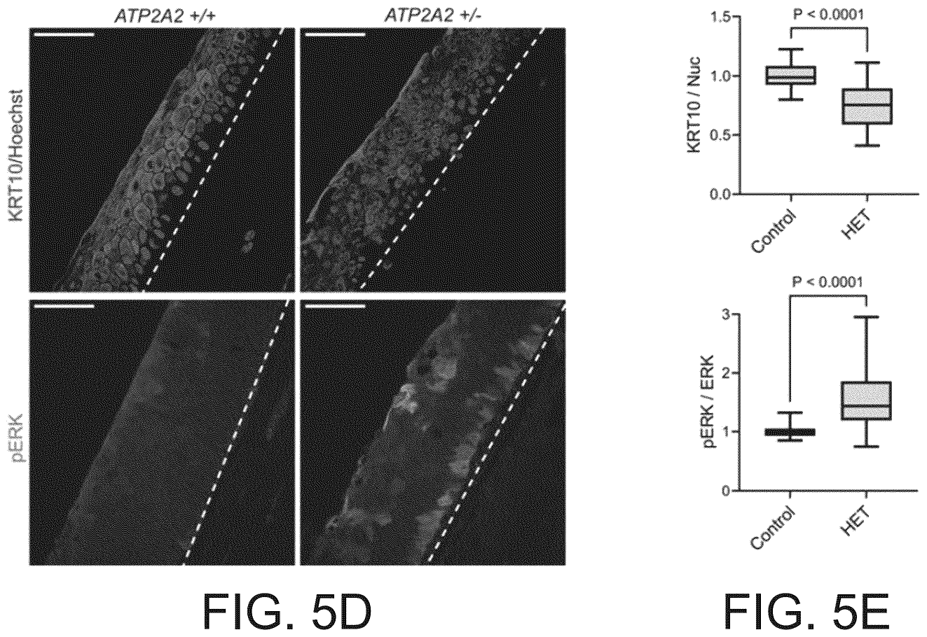

To determine if transcriptional changes in SERCA2-deficient keratinocytes translated to effects on protein expression, label-free mass spectrometry (MS)-based identification was performed of the proteome in two independent HET cell lines (FIG. 5A and Table 1). This technique confirmed deficiency of SERCA2, itself, while the levels of other ER-localized proteins (SEC61 translocon subunit beta (SEC61B), SEC61 translocon subunit alpha 1 (SEC61A1)) and an often-used housekeeping gene (glyceraldehyde-3-phosphate dehydrogenase (GAPDH)) were not significantly changed in HET cells compared to controls.

TABLE 1
Proteomic analysis.

| Gene | LFQ intensity | | | | | | Average | | p- |
|---|---|---|---|---|---|---|---|---|---|
| names | WT-1 | WT-2 | WT-3 | HET-1 | HET-2 | HET-3 | WT | HET | value |
| ALOX15B | 2.0E+06 | 1.9E+06 | 1.9E+06 | 0 | 0 | 0 | 1.9E+06 | 0 | 0.00 |
| ATP2A2 | 2.2E+07 | 2.2E+07 | 2.1E+07 | 1.2E+07 | 1.3E+07 | 1.3E+07 | 2.2E+07 | 1.3E+07 | 0.00 |
| DSG2 | 3.0E+07 | 2.8E+07 | 2.9E+07 | 3.1E+07 | 2.9E+07 | 2.8E+07 | 2.9E+07 | 2.9E+07 | 0.91 |
| DSG3 | 4.0E+07 | 3.6E+07 | 3.6E+07 | 2.8E+07 | 3.1E+07 | 3.2E+07 | 3.7E+07 | 3.0E+07 | 0.02 |
| DSP | 1.0E+08 | 1.0E+08 | 1.0E+08 | 8.8E+07 | 8.3E+07 | 8.8E+07 | 1.0E+08 | 8.7E+07 | 0.00 |
| GAPDH | 7.6E+08 | 7.5E+08 | 7.3E+08 | 8.7E+08 | 7.7E+08 | 8.3E+08 | 7.5E+08 | 8.2E+08 | 0.07 |
| IVL | 4.8E+07 | 4.5E+07 | 4.9E+07 | 2.0E+07 | 2.1E+07 | 1.7E+07 | 4.7E+07 | 2.0E+07 | 0.00 |
| JUP | 1.4E+08 | 1.3E+08 | 1.4E+08 | 1.4E+08 | 1.3E+08 | 1.2E+08 | 1.4E+08 | 1.3E+08 | 0.20 |
| KRT1 | 7.7E+07 | 1.7E+07 | 0 | 2.7E+07 | 2.0E+07 | 0 | 3.1E+07 | 1.6E+07 | 0.56 |
| KRT5 | 2.4E+09 | 2.7E+09 | 2.4E+09 | 2.5E+09 | 2.4E+09 | 2.0E+09 | 2.5E+09 | 2.3E+09 | 0.41 |
| KRT10 | 1.5E+07 | 1.5E+07 | 1.8E+07 | 6.9E+06 | 5.5E+06 | 3.6E+06 | 1.6E+07 | 5.3E+06 | 0.00 |
| KRT14 | 2.5E+09 | 2.5E+09 | 2.6E+09 | 2.6E+09 | 2.3E+09 | 2.3E+09 | 2.5E+09 | 2.4E+09 | 0.21 |
| KRT19 | 0 | 0 | 0 | 0 | 0 | 1.3E+08 | 0 | 4.4E+07 | 0.37 |
| NOTCH1 | 0 | 0 | 0 | 2.8E+06 | 0 | 2.8E+06 | 0.0E+00 | 1.9E+06 | 0.12 |
| PKP2 | 6.2E+06 | 6.4E+06 | 5.8E+06 | 5.8E+06 | 5.9E+06 | 5.9E+06 | 6.2E+06 | 5.9E+06 | 0.20 |
| PKP3 | 3.2E+06 | 4.0E+06 | 3.1E+06 | 0 | 4.0E+06 | 4.1E+06 | 3.4E+06 | 2.7E+06 | 0.61 |
| S100A7 | 7.6E+06 | 8.3E+06 | 1.0E+07 | 0 | 7.1E+05 | 6.6E+05 | 8.6E+06 | 4.6E+05 | 0.00 |
| S100A8 | 9.6E+07 | 9.1E+07 | 9.8E+07 | 5.8E+07 | 5.0E+07 | 7.3E+07 | 9.5E+07 | 6.0E+07 | 0.01 |
| S100A9 | 1.9E+08 | 1.7E+08 | 1.9E+08 | 8.3E+07 | 8.9E+07 | 1.0E+08 | 1.9E+08 | 9.2E+07 | 0.00 |
| SEC61A1 | 9.0E+06 | 8.8E+06 | 9.2E+06 | 1.0E+07 | 9.6E+06 | 7.7E+06 | 9.0E+06 | 9.3E+06 | 0.77 |
| SEC61B | 9.2E+06 | 7.6E+06 | 8.8E+06 | 9.8E+06 | 8.1E+06 | 9.8E+06 | 8.5E+06 | 9.2E+06 | 0.39 |
| SPRR3 | 1.1E+07 | 8.8E+06 | 1.2E+07 | 3.3E+06 | 3.5E+06 | 0 | 1.1E+07 | 2.3E+06 | 0.01 |
| TGM1 | 2.1E+07 | 2.2E+07 | 2.3E+07 | 1.5E+07 | 1.7E+07 | 1.4E+07 | 2.2E+07 | 1.5E+07 | 0.00 |

Table 1 provides the proteomic analysis results of ATP2A2 WT and HET cells in this example. Corresponding protein names for each gene are as follows: arachidonate 15-lipoxygenase B (ALOX15B), sarcoplasmic/endoplasmic reticulum calcium ATPase 2 (ATP2A2), desmoglein-2 (DSG2), desmoglein-3 (DSG3), desmoplakin (DSP), glyceraldehyde-3-phosphate dehydrogenase (GAPDH), Involucrin (IVL), junction plakoglobin (JUP), keratin, type II cytoskeletal 1 (KRT1), keratin, type II cytoskeletal 5 (KRT5), keratin, type I cytoskeletal 10 (KRT10), keratin, type I cytoskeletal 14 (KRT14), keratin, type I cytoskeletal 19 (KRT19), neurogenic locus notch homolog protein 1; notch 1 extracellular truncation; notch 1 intracellular domain (NOTCH1), plakophilin-2 (PKP2), plakophilin-3 (PKP3), protein S100-A7; protein S100-A7A (S100A7), protein S100-A8; protein S100-A8, n-terminally processed (S100A8), protein S100-A9 (S100A9), protein transport protein sec61 subunit alpha isoform 1 (SEC61A1), protein transport protein sec61 subunit beta (SEC61B), small proline-rich protein 3 (SPRR3), and protein-glutamine gamma-glutamyltransferase K (TGM1).

Proteomic analysis again revealed significant reduction in proteins that drive membrane lipid modifications (arachidonate 15-lipoxygenase type B (ALOX15B)) and several members of the S100 family of calcium-binding proteins. As well, HET cells had a deficiency of involucrin (IVL) and transglutaminase 1 (TGM1), two key regulators of the cornified cell envelope, a highly cross-linked protein meshwork assembled at the inner surface of differentiated keratinocytes that makes the outermost epidermal layers water-impermeable (Matsui T, Amagai M. Int Immunol. 2015; 27(6): p. 269-80; Crumrine D, et al. J Invest Dermatol. 2019; 139(4): p. 760-768). The TGM1 gene, in particular, has been linked to defective keratinocyte differentiation in patients with ARCI (Russell L J, et al. Nat Genet. 1995; 9(3): p. 279-83).

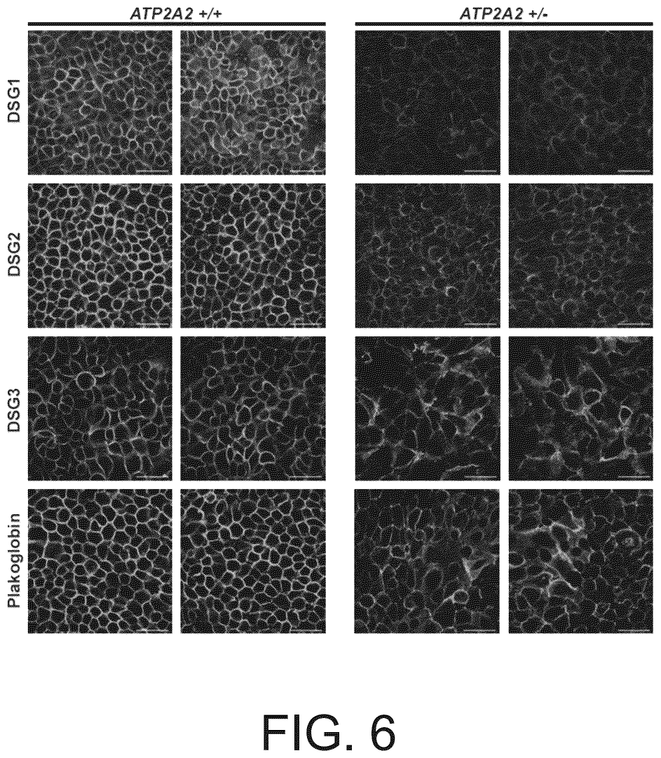

While many desmosome-associated proteins, including desmogleins (DSG2, DSG3), plakoglobin (JUP), desmoplakin (DSP), and plakophilins (PKP2, PKP3), exhibited little change in in SERCA2-deficient cells, their primary associated intermediate filament cytoskeletal component in the suprabasal epidermal layers, KRT10, was significantly reduced. If desmosomal complexes are not coupled to the keratin cytoskeleton, this dramatically compromises their ability to support intercellular adhesive strength (Huen A C, et al. J Cell Biol. 2002; 159(6): p. 1005-17). This finding was confirmed by immunoblotting HET and KO cell lysates, which exhibited reduced KRT10, while keratin 14 (KRT14) was not notably altered (FIG. 5B). If desmosomal complexes are not coupled to the keratin cytoskeleton, this dramatically compromises their ability to support intercellular adhesive strength (Huen A C, et al. J Cell Biol. 2002; 159(6): p. 1005-17). Consistent with their impaired cohesion in the mechanical dissociation assay, SERCA2-deficient keratinocytes displayed severely attenuated localization of desmosomal proteins to intercellular junctions compared with control cells (FIG. 6).

Informed by the proteomic data, it was determined if these alterations in SERCA2-deficient keratinocytes translated into pathogenic effects within a human tissue model. KO and HET cells were grown as organotypic skin cultures, which replicate the three-dimensional architecture of the stratified epidermis (Simpson C L, et al. Methods Mol Biol. 2010; 585: p. 127-46). Histologic analysis of epidermal cultures of HET cells revealed widening of intercellular spaces and marked disorganization of the upper keratinocyte layers undergoing the final stage of differentiation (FIG. 5C). Cornifying cells exhibited cytoplasmic vacuolization, impaired flattening, and retention of nuclei in the outermost keratinized layers (FIG. 5C, insets). These histologic defects occurred to a more extreme degree in KO cultures. While direct assessment of intercellular adhesive strength showed markedly reduced cohesion of SERCA2-deficient keratinocyte sheets (FIGS. 2C, 2D), organotypic cultures of these cells did not demonstrate full splitting between keratinocytes (also referred to as acantholysis), which may reflect differences in shear forces on the in vitro tissue model versus those experienced by the epidermis of patients, who exhibit trauma-induced blistering in areas of friction (Takagi A, et al. J Dermatol. 2016; 43(3): p. 275-9).

Consistent with the proteomic data, immunostaining SERCA2-deficient organotypic epidermis had reduced KRT10 in the suprabasal layers, which exhibited patchy expression of the cytoskeletal protein compared to the more uniform pattern seen in control cultures (FIGS. 5D, 5E, upper panels). Since KRT10 is a major regulator of epidermal integrity and differentiation (Porter R M, et al. J Cell Biol. 1996; 132(5): p. 925-36; Rothnagel J A, et al. Science. 1992; 257(5073): p. 1128-30; Reichelt J, Magin T M. J Cell Sci. 2002; 115(Pt 13): p. 2639-50), a concomitant reduction of KRT10 in SERCA2-depleted epidermis could be responsible for the impaired intercellular adhesion and abnormal cornification seen in HET organotypic epidermis, which overlap with the pathologic features seen in DD biopsies.

It has been previously shown that KRT10 expression is disrupted upon epidermal hyperactivation of MEK and ERK in the MAPK pathway (Harmon R M, et al. J Clin Invest. 2013; 123(4): p. 1556-70; Scholl F A, et al. Cancer Res. 2004; 64(17): p. 6035-40; Getsios S, et al. J Cell Biol. 2009; 185(7): p. 1243-58). In fact, rises in cytoplasmic calcium, known to drive keratinocyte differentiation (Bikle D D, et al. Expert Rev Endocrinol Metab. 2012; 7(4): p. 461-472; Menon G K, et al. J Invest Dermatol. 1985; 84(6): p. 508-12), have been shown to trigger mitogen-independent MAPK signaling, which can be augmented by SERCA2 blockade (Schmidt M, et al. J Biol Chem. 2000; 275(52): p. 41011-7). Further connecting this signaling pathway to DD, a DD-like disorder called Grover disease, characterized by similar defects in epidermal adhesion and keratinocyte cornification, has been linked to aberrant activation of MEK in patients (Carlos G, et al. JAMA Dermatol. 2015; 151(10): p. 1103-9; See S H C, et al. J Cutan Pathol. 2019; 46(1): p. 6-15). These observations coupled with our transcriptomic and proteomic analyses indicates that SERCA2 deficiency leads to aberrant activation of the MAPK pathway, which represents a druggable target for treating DD (Fu L, et al. J Med Chem. 2022; 65(20): p. 13561-13573).

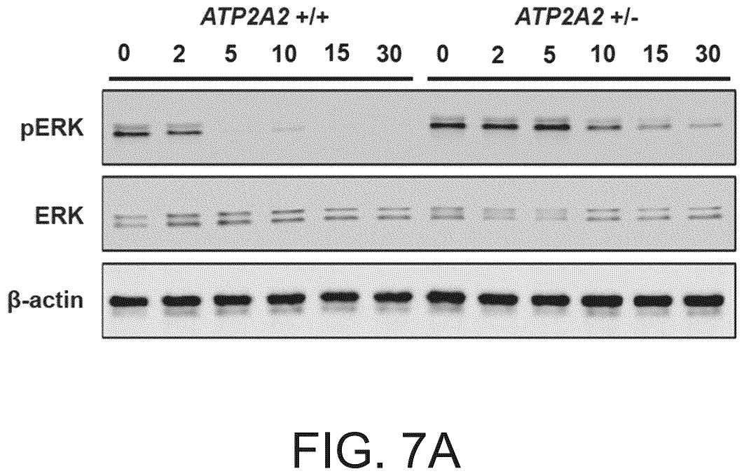

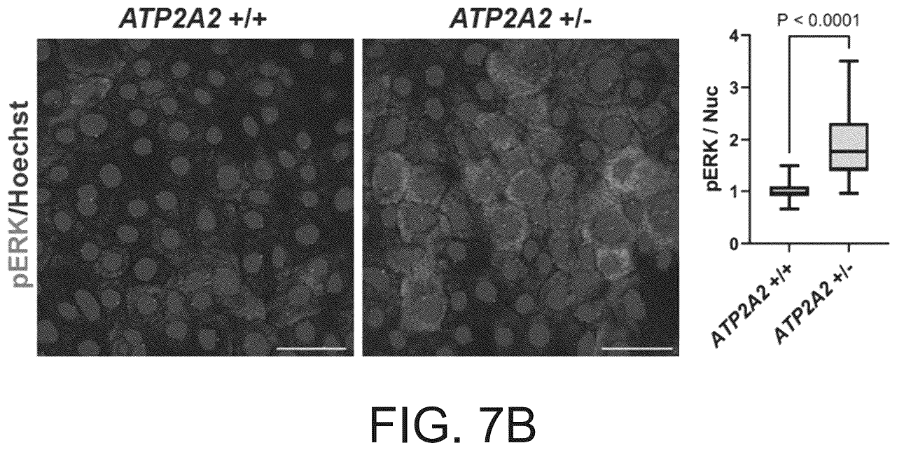

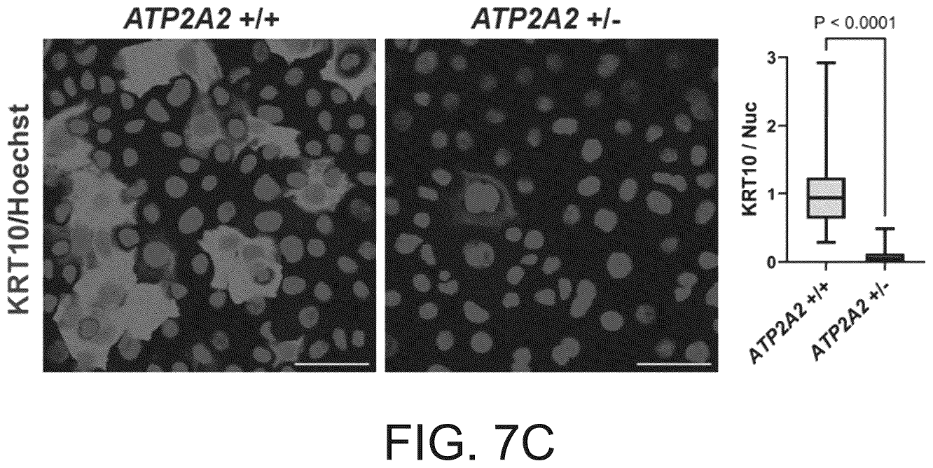

Thus, it was assessed whether hyper-activation of MEK, which functions in the MAPK pathway by activating extracellular-signal regulated kinase (ERK) via phosphorylation, could explain the defects in adhesion and differentiation seen in the disclosed DD tissue model. SERCA2-deficient keratinocytes displayed prolonged ERK activation compared with control cells following short-term exposure to increased extracellular calcium (FIG. 7A). Reflecting longer term MEK hyperactivation in SERCA2-deficient keratinocytes, it was found, by immunostaining, that pERK was significantly elevated in confluent monolayers and mature organotypic epidermal cultures of HET cells compared with controls (FIGS. 5D, 5E, lower panels; and FIG. 7B). In both monolayer and organotypic cultures, pERK levels were inversely correlated with the expression of KRT10 (FIGS. 5D, 5E, upper panels; and FIG. 7C).

### Chemical Inhibition of SERCA2 in Keratinocytes and Organotypic Epidermis Replicates Features of DD Pathology and Induces ERK Activation.

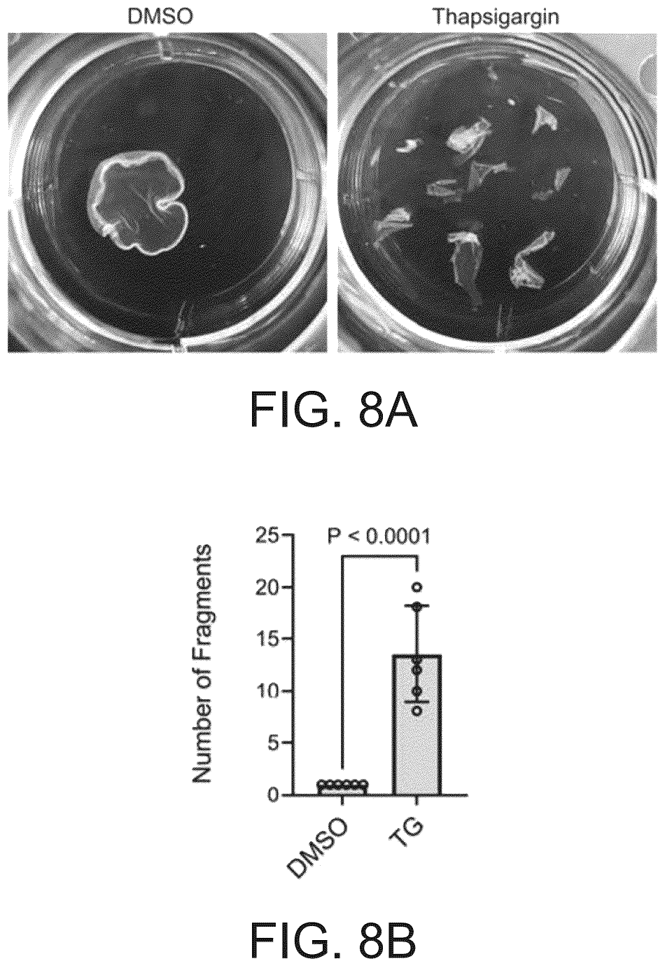

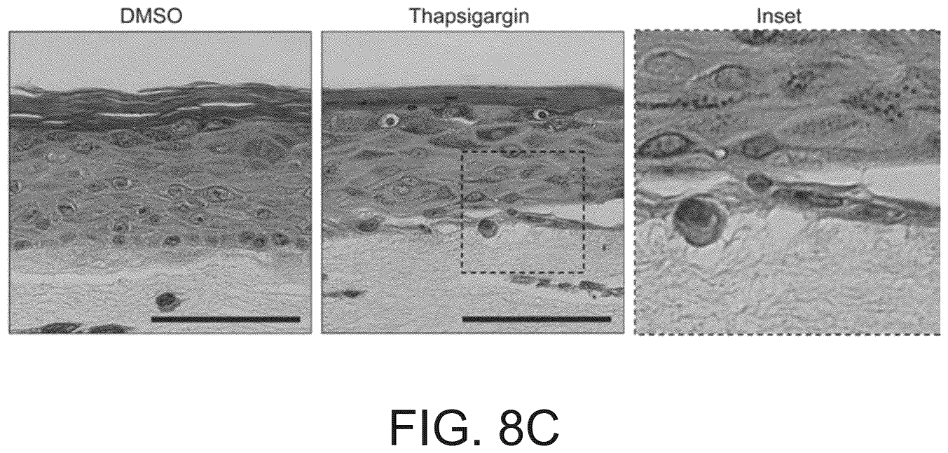

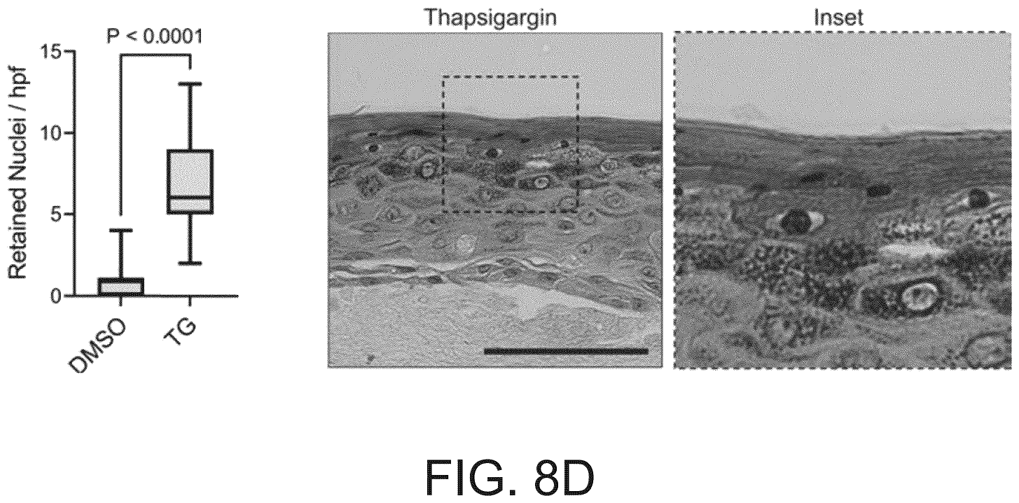

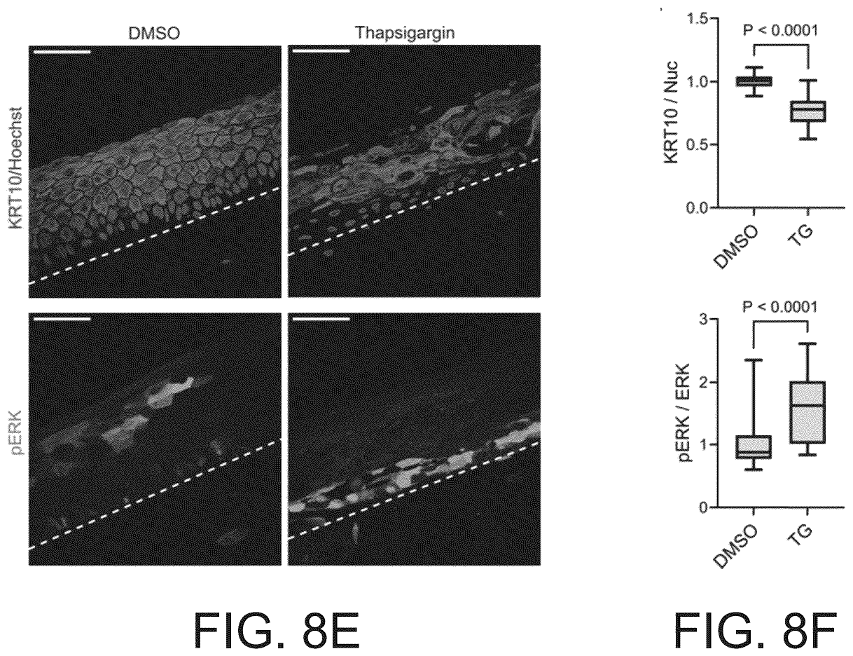

To complement the disclosed results in SERCA2-deficient immortalized keratinocytes, the effect of a chemical inhibitor of SERCA2, thapsigargin (TG), on normal human epidermal keratinocytes (NHEKs) was assessed. Treatment of NHEK monolayers with TG caused a significant increase in epithelial sheet fragmentation (FIGS. 8A, 8B), which confirms the role of SERCA2 in promoting intercellular adhesion in primary human keratinocytes. Building on these findings, mature organotypic epidermis grown from NHEKs were treated with TG, which disrupted keratinocyte cohesion most notably between basal and suprabasal cell layers (FIG. 8C, inset), a pathologic feature of DD (See S H C, et al. J Cutan Pathol. 2019; 46(1): p. 6-15). Chemical inhibition of SERCA2 also disrupted keratinocyte differentiation causing retention of nuclei in the cornified layers (FIG. 8D, inset), a feature consistent with aberrant epidermal maturation (termed dyskeratosis) seen in DD (See S H C, et al. J Cutan Pathol. 2019; 46(1): p. 6-15; Hovnanian A. Biochem Biophys Res Commun. 2004; 322(4): p. 1237-44).

Supporting the disclosed data from both RNA sequencing and proteomic analysis, TG-treated organotypic epidermis exhibited a reduction in KRT10 with immunostaining of tissue cross-sections demonstrating patchy loss of this cytoskeletal element in the suprabasal layers (FIGS. 8E, 8F, upper panels). Disruption of KRT10 function can compromise epidermal integrity and cause abnormal cornification as in patients with KRT10 mutations in epidermolytic ichthyosis (Rothnagel J A, et al. Science. 1992; 257(5073): p. 1128-30; Huber M, et al. J Invest Dermatol. 1994; 102(5): p. 691-4; Pulkkinen L, et al. J Clin Invest. 1993; 91(1): p. 357-61). Like in SERCA2-deficient keratinocytes, ERK hyper-activation was also seen upon TG treatment of NHEK epidermal cultures; pERK was highest in the lower keratinocyte layers in this example, where intercellular adhesion was most compromised (FIGS. 8E, 8F, lower panels). These findings indicate that loss of SERCA2 function disrupted tissue integrity and epidermal differentiation though over-activation of the MAPK pathway via ERK, resulting in depletion of KRT10.

### Biopsies of Darier Disease Exhibit Mis-Localization of Cadherins, Loss of Keratin 10, and Elevated ERK Activation.

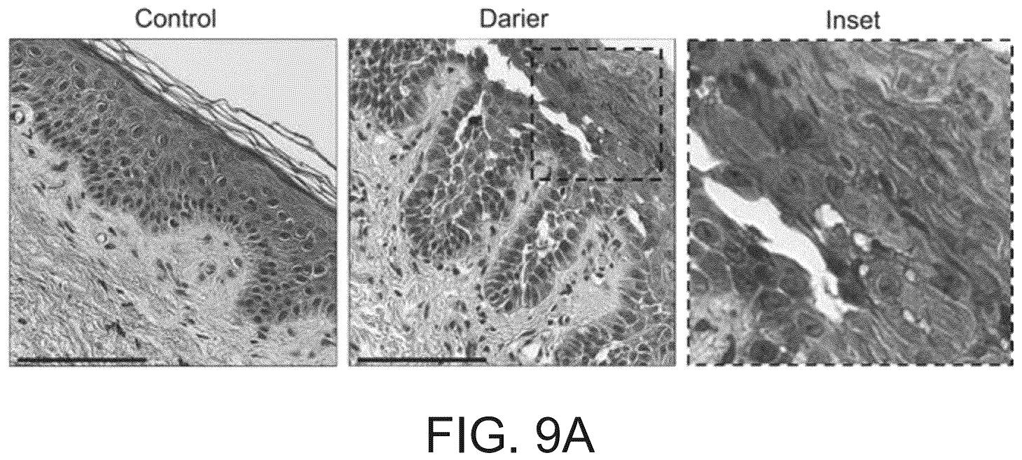

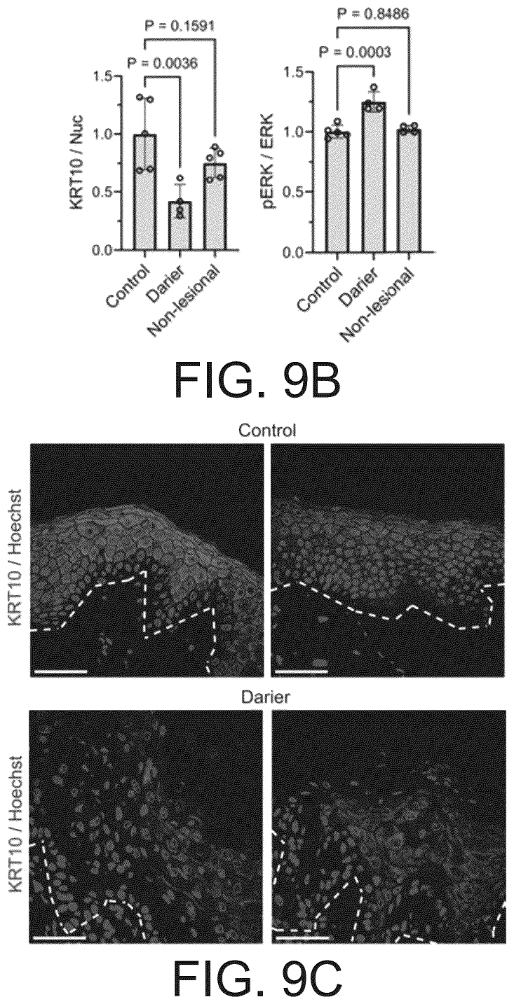

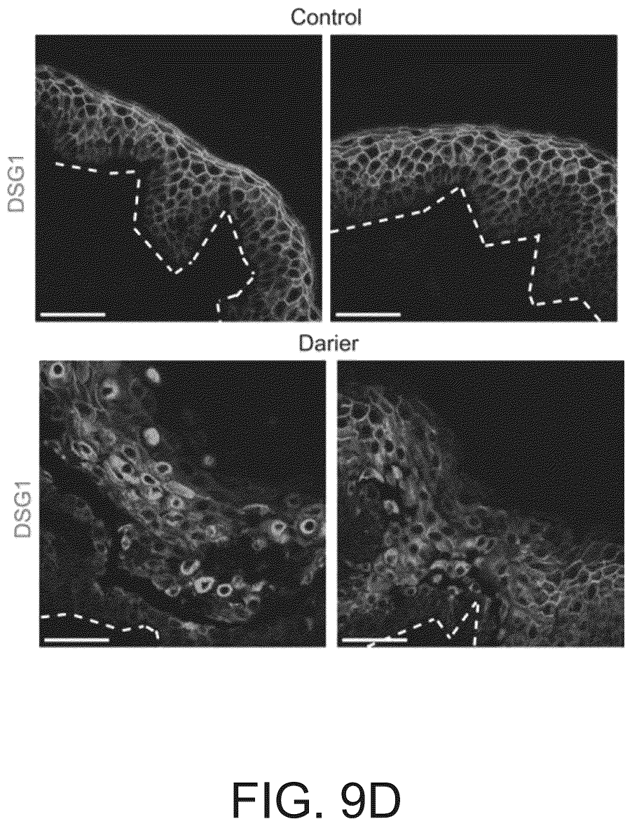

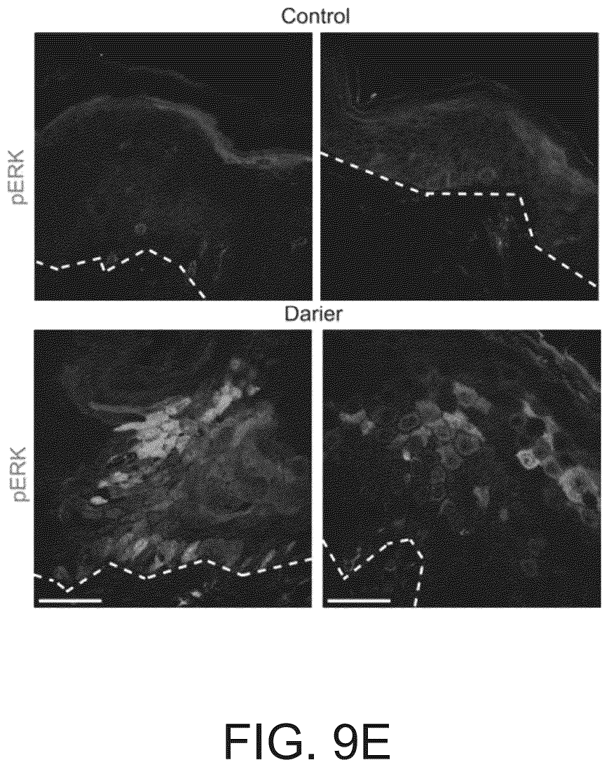

To directly test if the in vitro data from SERCA2-depleted or -inhibited epidermal cultures were reflective of DD pathogenesis, de-identified skin biopsies from patients with DD were examined. Similar to the pathologic features, it was found that, in organotypic cultures having SERCA2 genetically ablated or chemically inhibited, histologic examination of DD biopsies demonstrated loss of keratinocyte cohesion and defective differentiation with retention of nuclei in the cornified layers (FIG. 9A, inset). Immunostaining biopsies from five DD patients compared to normal control skin demonstrated a significant reduction in KRT10 and a concomitant increase in ERK activation within lesional skin (FIG. 9B), consistent with the findings from the in vitro human tissue model. Like in organotypic epidermis, analysis of immunostained DD biopsy cross-sections revealed a significant reduction in overall KRT10 intensity with patchy loss of the cytoskeletal element within DD lesions (FIG. 9C).

In addition, it was found that protein levels of DSG1, the primary cadherin mediator of desmosomal adhesion in the upper epidermal layers (Simpson C L, Green K J. J Invest Dermatol. 2007; 127(E1): p. E15-6; Hammers C M, Stanley J R. J Clin Invest. 2013; 123(4): p. 1419-22), were modestly reduced (data not shown). However, the localization of this cadherin was severely disrupted in DD lesions (FIG. 9D), collapsing around the nucleus instead of being concentrated at intercellular junctions. These findings agree with prior in vitro studies showing that keratinocytes having SERCA2 depleted by RNA interference retained normal levels of desmosomal components but exhibited major defects in trafficking of these adhesive building blocks to the plasma membrane, thus impairing the cells' ability to assemble strong intercellular junctions and compromising monolayer integrity (Hobbs R P, et al. FASEB J. 2011; 25(3): p. 990-1001; Savignac M, et al. Biochim Biophys Acta. 2011; 1813(5): p. 1111-7; Dhitavat J, et al. J Invest Dermatol. 2003; 121(6): p. 1349-55; Mauro T. J Invest Dermatol. 2014; 134(7): p. 1800-1801).

ERK activation assessed by immunostaining pERK, on the other hand, was significantly increased in biopsies from patients with DD compared to biopsies of normal skin (FIG. 9E). These data from DD tissue support the findings from SERCA2-depleted or -inhibited human epidermal cultures and bolster the disclosed model in which hyper-activation of the MAPK pathway via ERK is a pathogenic driver in DD and represents a potential therapeutic target.

### Keratin Expression and Adhesive Integrity in SERCA2-Deficient Keratinocytes are Rescued by MEK Inhibitors.

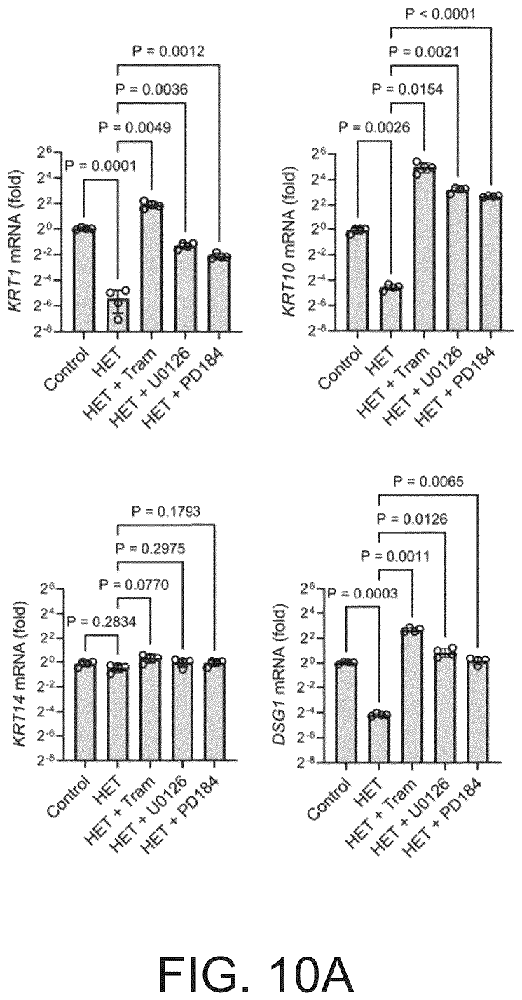

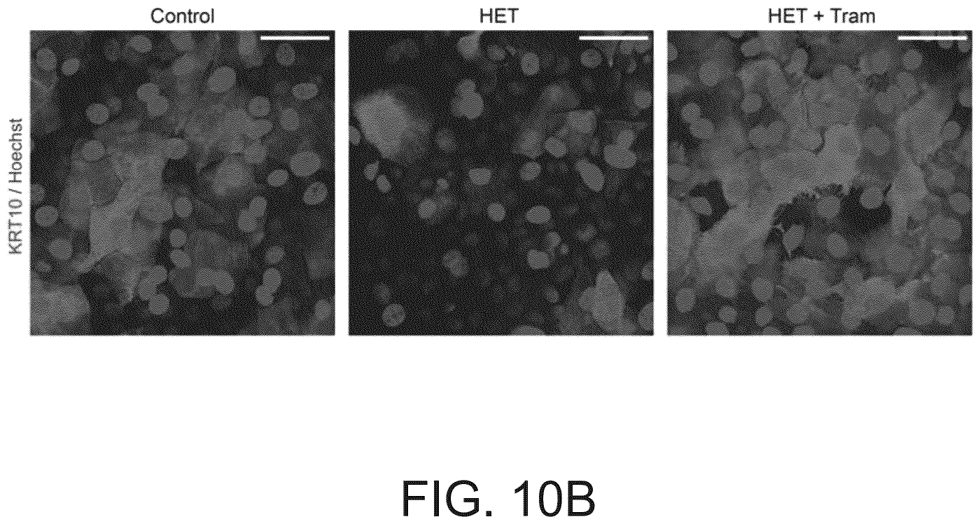

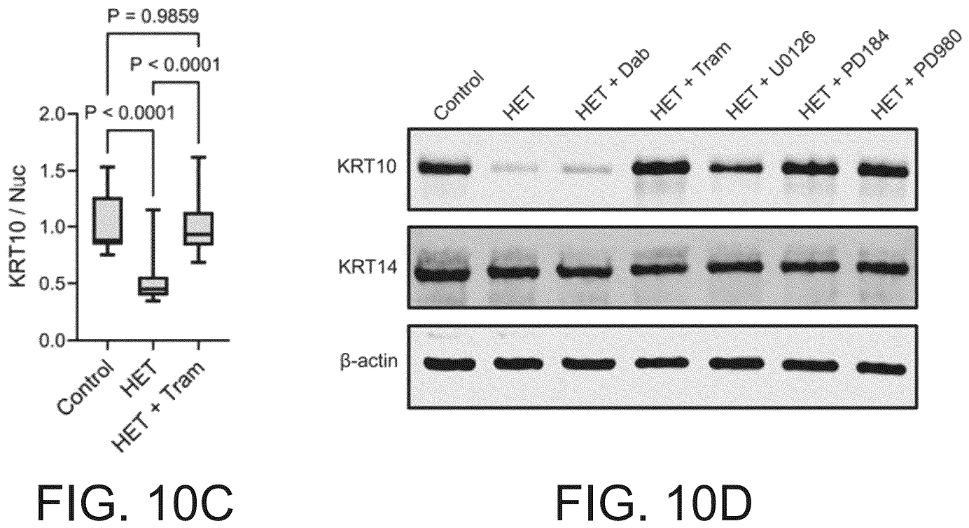

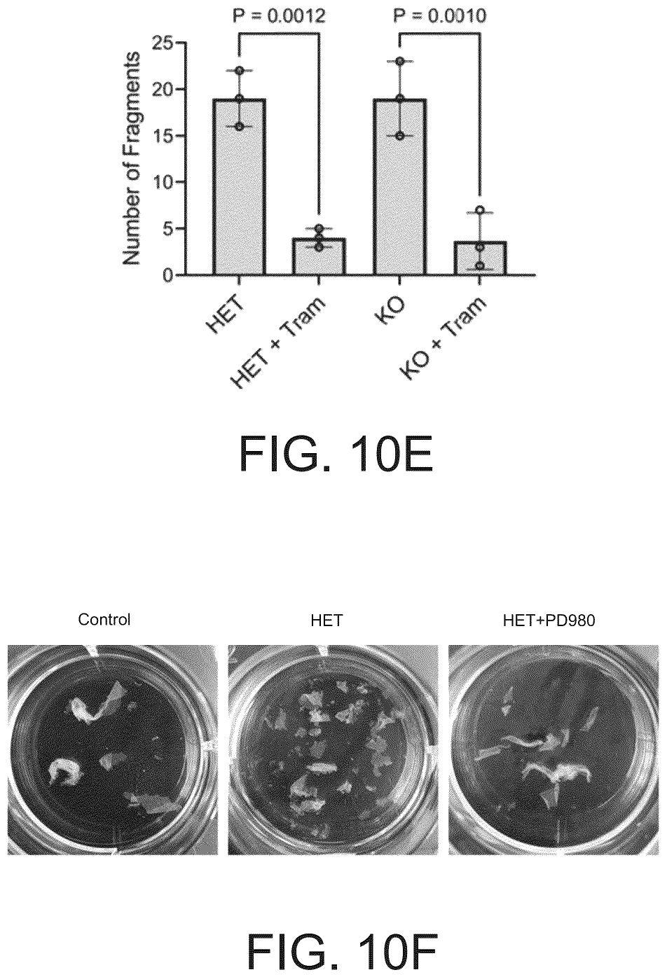

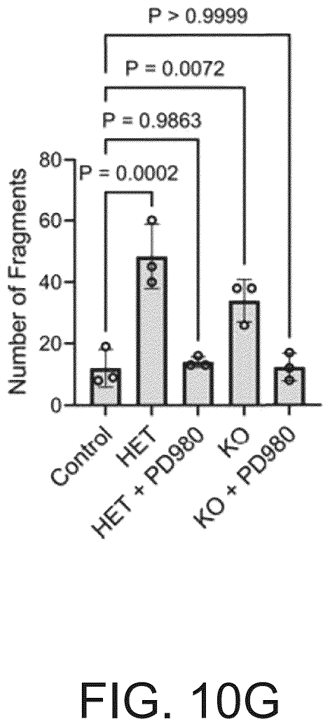

The effect of multiple selective MEK inhibitors on SERCA2-depleted keratinocytes was assessed to determine if elevated MEK activity directly contributes to the phenotype of SERCA2 deficiency. Quantification of keratinocyte mRNA levels by RT-PCR again confirmed that HET cells exhibit significantly reduced expression of the suprabasal keratins, KRT1 and KRT10, and DSG1 while the mRNA encoding KRT14 was not significantly different from controls (FIG. 10A). Treatment of HET cells with any of three MEK inhibitors (trametinib, U0126, PD184352 (PD184)) was sufficient to augment expression of KRT1, KRT10, and DSG1, while they did not significantly alter KRT14 mRNA levels.

These differences in mRNA translated to changes in protein levels with HET cells exhibiting significantly reduced KRT10 as shown by immunostaining differentiated keratinocyte cultures (FIG. 10B). Consistent with its effect on Krt10 mRNA, MEK inhibition increased KRT10 protein, making its levels comparable to that of control cells (FIG. 10C). Assessing keratinocyte lysates by immunoblot similarly demonstrated a reduction in KRT10, which multiple individual MEK inhibitors restored to near the baseline level of controls (FIG. 10D). In contrast, KRT14 levels were comparable in control and HET cells and were not appreciably altered by MEK inhibitors in this example. Targeting the MAPK pathway upstream of MEK using dabrafenib to inhibit RAF failed to rescue KRT10 expression in this example. This result is consistent with prior data indicating that chronic suppression of RAF leads to paradoxical downstream elevation in MEK activity (Poulikakos P I, et al. Nature. 2010; 464(7287): p. 427-30). This unintended result of RAF blockade changed the standard-of-care for patients with RAF-driven cancers to include dual RAF and MEK inhibitors to mitigate adverse effects of RAF inhibitors (Carlos G, et al. JAMA Dermatol. 2015; 151(10): p. 1103-9; Flaherty K T, et al. N Engl J Med. 2012; 367(18): p. 1694-703). In fact, MEK hyper-activation in patients treated with RAF inhibitor monotherapy can actually cause a DD-like skin eruption with biopsies showing impaired keratinocyte adhesion and defective epidermal cornification (Awe O, et al. Int J Dermatol. 2022; 61(5): p. 591-594; Anforth R, et al. Lancet Oncol. 2013; 14(1): p. e11-8; Babuna Kobaner G, et al. Australas J Dermatol. 2018; 59(3): p. e231-e233; Chu E Y, et al. J Am Acad Dermatol. 2012; 67(6): p. 1265-72).

Since trametinib robustly rescued the expression of adhesive proteins in HET cells and is an FDA-approved drug for the treatment of RAF-mutated cancers (Flaherty K T, et al. N Engl J Med. 2012; 367(2): p. 107-14; Roskoski R, Jr. Pharmacol Res. 2019; 142: p. 151-168), it was investigated whether this compound could augment intercellular adhesion in SERCA2-deficient keratinocytes. Treatment with trametinib enhanced the resistance of HET or KO keratinocyte sheets to disruption by mechanical stress (FIG. 10E). An additional MEK inhibitor PD98059 (PD980) similarly rescued cohesion of SERCA2-depleted cultures (FIGS. 10F, 10G), indicating the clinical utility of inhibiting this kinase to treat DD.

MEK Inhibitors Promote Keratinocyte Cohesion and Mitigate Epidermal Tissue Disruption from SERCA2 Inhibition.

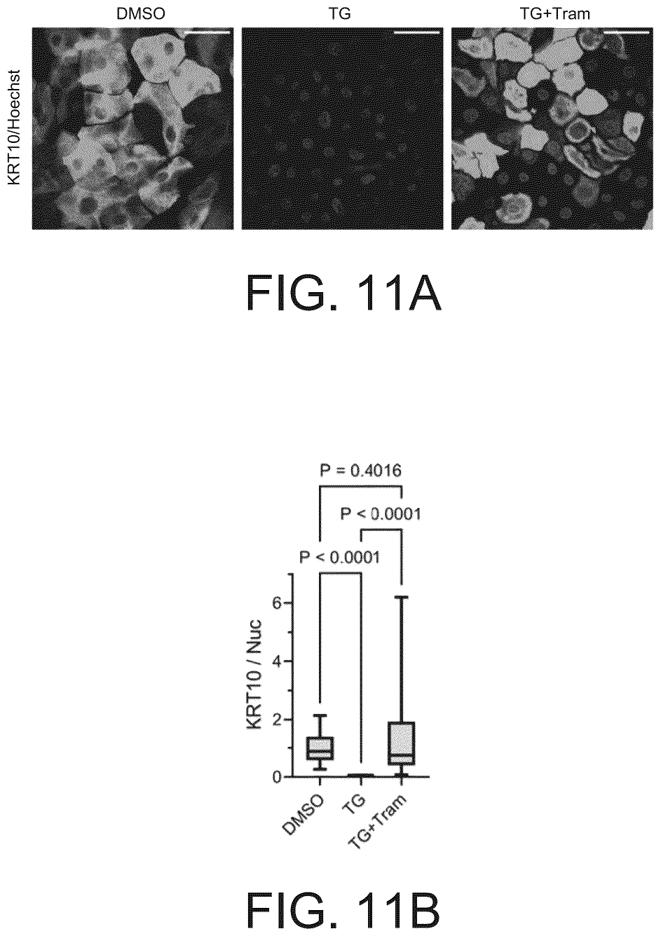

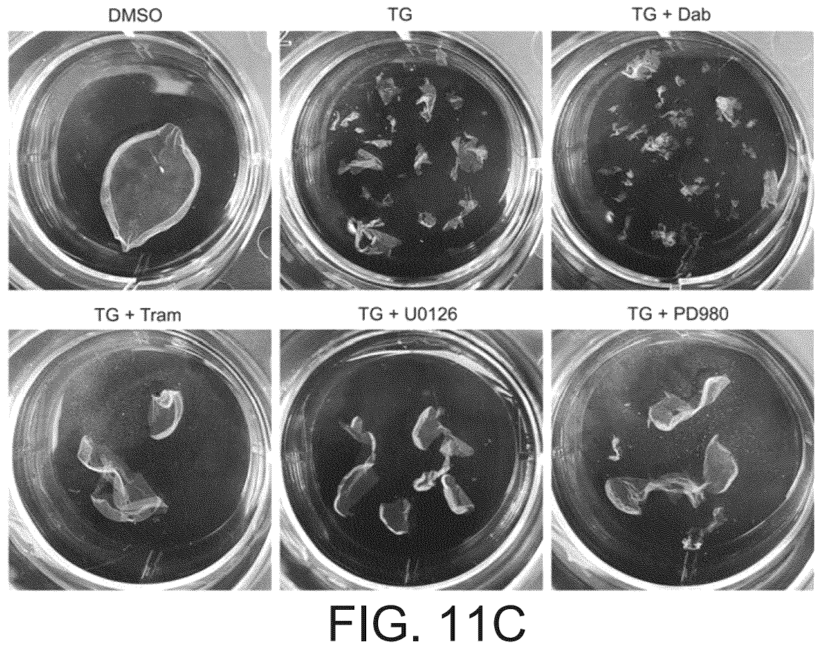

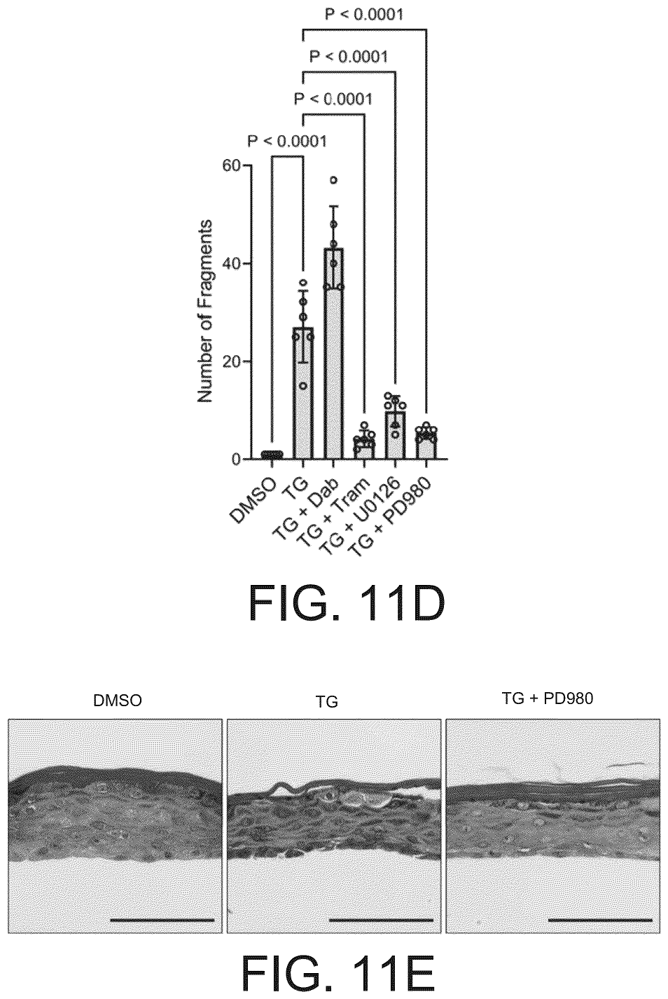

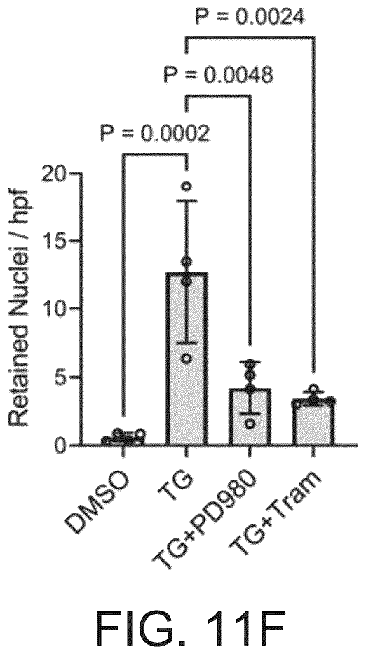

To confirm that ERK hyper-activation drives the pathogenic effects of reduced SERCA2 function, it was determined whether MEK inhibitors could reverse TG-induced loss of keratinocyte adhesion and impaired epidermal differentiation. Treatment of NHEKs with TG suppressed their ability to up-regulate KRT10 as indicated by immunostaining differentiated keratinocytes (FIGS. 11A, 11B) and this effect of TG was mitigated by concomitant treatment with trametinib to inhibit MEK.

This effect of MEK suppression on KRT10 in SERCA2-inhibited keratinocytes translated into a restoration of intercellular adhesive strength. While TG-treated NHEK monolayers readily fragmented upon mechanical stress, the integrity of epithelial sheets was restored by MEK inhibition with trametinib, U0126, or PD98059 (PD980) despite SERCA2 inhibition (FIGS. 11C, 11D). In contrast, upstream inhibition of RAF with dabrafenib did not promote intercellular adhesion, but rather exacerbated the effect of TG, further reducing the integrity of NHEK monolayers. These results are consistent with the reported ability of dabrafenib monotherapy to cause paradoxical hyper-activation of MEK (Poulikakos P I, et al. Nature. 2010; 464(7287): p. 427-30), which, based on the findings, appears to drive the pathogenic effects of SERCA2 deficiency and explains the similar findings in biopsies of RAF inhibitor-induced Grover disease and DD.

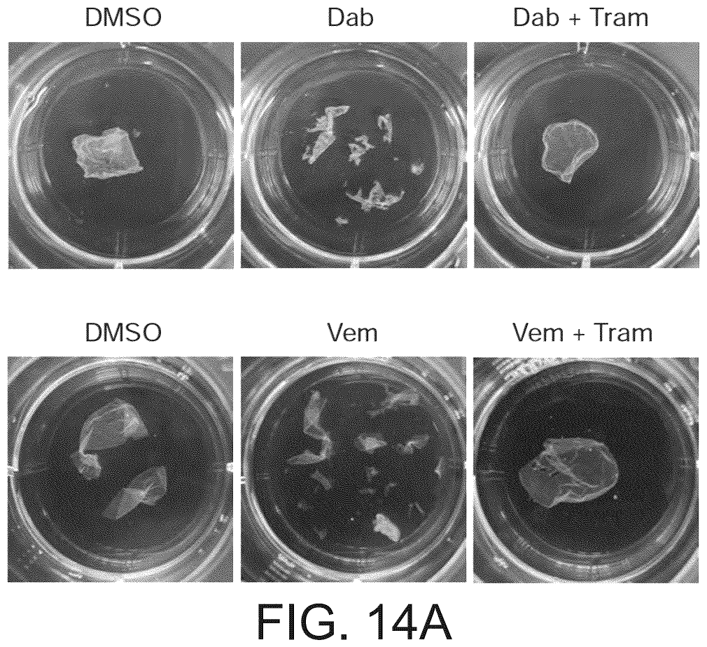

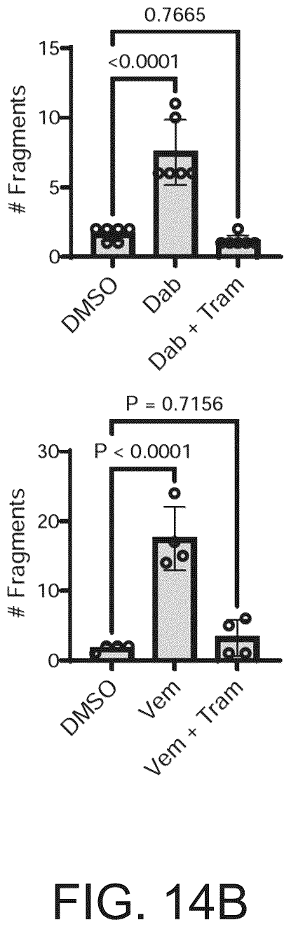

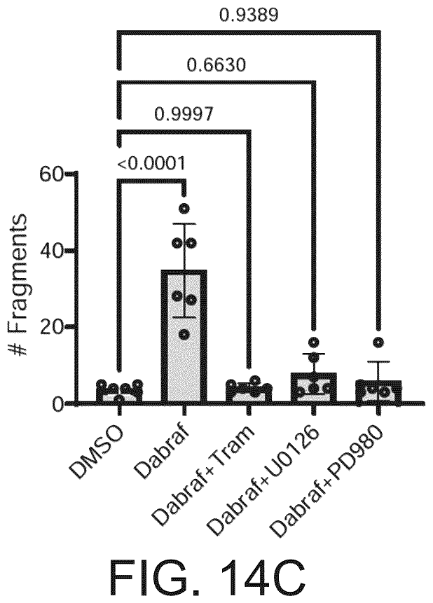

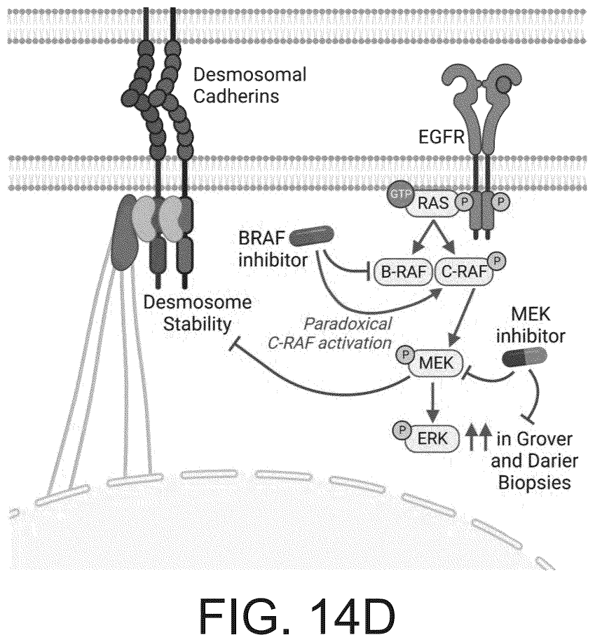

Mature organotypic epidermis were treated with TG either alone or with PD98059 or trametinib to determine if MEK inhibition could prevent DD-like pathologic changes in organotypic epidermis. While TG-treated cultures exhibited loss of keratinocyte cohesion within the epidermal tissue, this effect was mitigated by MEK inhibitors (FIG. 11E). Moreover, treatment with PD98059 (PD980) or trametinib reduced TG-induced retention of nuclei in the cornified layers (FIG. 11F), indicating that MEK inhibition could normalize epidermal differentiation in DD. Together, these data support the disclosed model in which the pathogenic effects of SERCA2 loss-of-function in epidermis are driven by ERK hyper-activation, which can be mitigated by MEK inhibition (see Graphical Abstract, FIG. 14D).

### Discussion

The advent of rational molecular therapeutics has revolutionized the treatment of many dermatologic conditions (Silverberg N. Clin Dermatol. 2020; 38(4): p. 462-466; Hill C R, Theos A. Dermatol Clin. 2019; 37(2): p. 229-239); however, most advances have been made for common inflammatory diseases, like psoriasis and atopic dermatitis, driven by soluble cytokines amenable to targeting by monoclonal antibodies (David E, et al. Clin Exp Allergy. 2023; 53(2): p. 156-172; Lin C P, et al. Curr Opin Pharmacol. 2022; 67: p. 102292). Unfortunately, skin blistering diseases and disorders of cornification are rare, and most lack any FDA-approved treatments, though recent work suggests cytokine targeting may improve certain subtypes of ichthyosis (Lefferdink R, et al. Arch Dermatol Res. 2022; Kim M, et al. J Invest Dermatol. 2022; 142(9): p. 2363-2374 e18; Paller A S, et al. J Allergy Clin Immunol. 2017; 139(1): p. 152-165). While an inflammatory signature in the disclosed keratinocyte cultures was not identified, SERCA2-deficient cells had reduced IF127 and other interferon-response genes, which might explain the susceptibility of DD lesions to viral infection (Vogt K A, et al. J Am Acad Dermatol. 2015; 72(3): p. 481-4). For DD, the knowledge of its genetic etiology has not significantly advanced the treatment of this chronic, incurable disorder. Though retinoids, which modulate keratinocyte differentiation, may be helpful in DD (Hanna N, et al. J Cutan Med Surg. 2022; 26(3): p. 280-290; Christophersen J, et al. Acta Derm Venereol. 1992; 72(2): p. 150-2), they are potent teratogens and can cause multi-system toxicity with long-term use (Zaenglein A L, et al. Pediatr Dermatol. 2021; 38(1): p. 164-180; Doolan B J, et al. Clin Exp Dermatol. 2022; 47(12): p. 2273-2276). Some evidence indicates reducing ER stress may be a viable alternative approach for DD treatment (Savignac M, et al. J Invest Dermatol. 2014; 134(7): p. 1961-1970), though this strategy has not yet been tested clinically.

Using mice as a pre-clinical model has enabled discoveries about disease pathogenesis that translated into new therapies for patients, but animal models do not always phenocopy human pathology, as with disrupting Atp2a2 in mice (Prasad V, et al. Cancer Res. 2005; 65(19): p. 8655-61; Toki H, et al. Biochem Biophys Res Commun. 2016; 476(4): p. 175-182). The development of organoid models to replicate human tissues in vitro has the potential to revolutionize investigating disease pathogenesis, identifying lead compounds, and screening for therapeutic efficacy and toxicity (Wang H, et al. Clin Transl Sci. 2021; 14(5): p. 1659-1680; Haase K, Freedman B S. Development. 2020; 147(9)). In this example, gene editing is leveraged in human keratinocytes and an organotypic epidermal model to elucidate the pathogenic mechanisms underlying a rare genetic disorder, which allowed identification of therapeutic avenues. The disclosed findings support the use of organoids to generate large-scale transcriptomic and proteomic datasets that can be mined for rational drug targets to validate both in vitro and in patient-derived tissues (Wang H, et al. Clin Transl Sci. 2021; 14(5): p. 1659-1680; Kdimati S, et al. Methods Mol Biol. 2023; 2589: p. 111-126; Wu W, Li X, Yu S. Acta Biomater. 2022; 146: p. 23-36; Novelli G, et al. Front Cell Dev Biol. 2022; 10: p. 1059579; LeSavage B L, et al. Nat Mater. 2022; 21(2): p. 143-159).

While the primary DD pathology derives from impaired ER calcium homeostasis (Savignac M, et al. Biochim Biophys Acta. 2011; 1813(5): p. 1111-7; Hovnanian A. Biochem Biophys Res Commun. 2004; 322(4): p. 1237-44), direct targeting of SERCA2 is therapeutically challenging. Recent intravital imaging of murine epidermis revealed that calcium flux undergoes dramatic shifts during epidermal proliferation (Moore J. L. et al. bioRxiv. 2021: p. 10.12.464066) and in the late stages of cornification (Matsui T, et al. Proc Natl Acad Sci USA. 2021; 118(17)). Thus, sustained drug-induced activation of SERCA2 comprises the dynamic nature of calcium-mediated signals critical for epidermal functions (Bikle D D, et al. Expert Rev Endocrinol Metab. 2012; 7(4): p. 461-472; Elsholz F, et al. Eur J Dermatol. 2014; 24(6): p. 650-61). Since calcium is a major second messenger regulating diverse biological functions including skeletal muscle contraction, cardiac pacing, neurotransmitter release, and apoptosis in many cell types (MacLennan DH. Eur J Biochem. 2000; 267(17): p. 5291-7; Britzolaki A, et al. Adv Exp Med Biol. 2020; 1131: p. 131-161), compounds affecting intracellular calcium have a narrow therapeutic window. In fact, coincidence of neuropsychiatric and cardiac disease with DD (Jacobsen N J, et al. Hum Mol Genet. 1999; 8(9): p. 1631-6; Gordon-Smith K, et al. Am J Med Genet B Neuropsychiatr Genet. 2018; 177(8): p. 717-726) indicates a role beyond the skin for SERCA2 (Bachar-Wikstrom E, Wikstrom J D. Acta Derm Venereol. 2021; 101(4): p. adv00430), including in higher-order cognition and behavior (Nakajima K, et al. Hum Mol Genet. 2021; 30(18): p. 1762-1772; Hua P T, Caplan J P. Psychosomatics. 2020; 61(3): p. 281-283; Li Pomi F, et al. Clin Case Rep. 2021; 9(6): p. e04263).

The study of rare diseases can yield unexpected findings that improve the understanding of fundamental biology or the pathology of more common disorders (Mao X, et al. J Invest Dermatol. 2014; 134(1): p. 68-76). While this example focused on DD, it was found that MEK inhibition robustly increased expression of KRT1 and KRT10. Given that multiple MEK inhibitors are approved for clinical use (Fu L, et al. J Med Chem. 2022; 65(20): p. 13561-13573; Flaherty K T, et al. N Engl J Med. 2012; 367(2): p. 107-14; Berkowitz P, et al. J Invest Dermatol. 2008; 128(3): p. 738-40), these could be evaluated for treating skin fragility disorders due to defective keratins like epidermolytic ichthyosis and epidermolysis bullosa simplex, linked to KRT1/KRT10 and KRT5/KRT14 mutation, respectively (Pulkkinen L, et al. J Clin Invest. 1993; 91(1): p. 357-61: Berkowitz P, et al. Proc Natl Acad Sci USA. 2006; 103(34): p. 12855-60; Egu D T, et al. Br J Dermatol. 2020; 182(4): p. 987-994). MEK inhibitors augment expression of wild-type KRT1 and KRT10 protein, which indicates that these compounds can rescue keratinocyte integrity in these orphan diseases. Likewise, increasing DSG1 levels with MEK inhibitors could be therapeutic for DSG1-linked disorders such as palmoplantar keratoderma. The disclosed results demonstrating ERK hyper-activation in DD bring to mind a non-inherited dermatologic disorder called Grover disease, which can be induced by BRAF inhibition (Awe O, et al. Int J Dermatol. 2022; 61(5): p. 591-594; Anforth R, et al. Lancet Oncol. 2013; 14(1): p. e 11-8; Chu E Y, et al. J Am Acad Dermatol. 2012; 67(6): p. 1265-72). Biopsy findings in Grover disease can be indistinguishable from DD (See S H C, et al. J Cutan Pathol. 2019; 46(1): p. 6-15), which indicates that they share a common pathogenic mechanism. The fact that BRAF inhibitor-induced Grover disease is suppressed by adding a MEK inhibitor (Carlos G, et al. JAMA Dermatol. 2015; 151(10): p. 1103-9; Flaherty K T, et al. N Engl J Med. 2012; 367(18): p. 1694-703) further indicates that MEK inhibitors provide an effective therapeutic for DD.

The disclosed findings indicate that excess ERK signaling disrupts the differentiation and cohesion of epidermal keratinocytes (Harmon R M, et al. J Clin Invest. 2013; 123(4): p. 1556-70; Egu D T, et al. Front Immunol. 2019; 10: p. 2883; Khavari T A, Rinn J. Cell Cycle. 2007; 6(23): p. 2928-31; Getsios S, et al. J Cell Biol. 2009; 185(7): p. 1243-58) to drive the pathogenesis of DD, which can be reversed by inhibiting MAPK/ERK signaling through MEK. Extensive data previously implicated a pathogenic role for the MAPK pathway in pemphigus via p38 (Mao X, et al. J Invest Dermatol. 2014; 134(1): p. 68-76; Berkowitz P, et al. J Invest Dermatol. 2008; 128(3): p. 738-40; Berkowitz P, et al. Proc Natl Acad Sci USA. 2006; 103(34): p. 12855-60), which did not progress to clinical approval (Egu D T, et al. Br J Dermatol. 2020; 182(4): p. 987-994). However, given that multiple MEK inhibitors are already approved for clinical use with long-term data supporting their safety (Fu L, et al. J Med Chem. 2022; 65(20): p. 13561-13573; Flaherty K T, et al. N Engl J Med. 2012; 367(18): p. 1694-703; Flaherty K T, et al. N Engl J Med. 2012; 367(2): p. 107-14; Falchook G S, et al. Lancet Oncol. 2012; 13(8): p. 782-9), the disclosed findings indicate that MEK inhibition represents a therapeutic strategy for both inherited and acquired blistering diseases, skin fragility syndromes, keratodermas, and disorders of cornification, all of which are in great need of better treatments.

### Second Experimental Example

### Introduction

Grover disease is an acquired dermatologic disorder characterized by pruritic vesicular and eroded skin lesions. While the pathologic features of Grover disease are well-defined, including impaired cohesion of epidermal keratinocytes, the etiology of Grover disease remains unclear, and the disease lacks any U.S. Food and Drug Administration (FDA)-approved therapy. Drug-induced Grover disease occurs in patients treated with proto-oncogene B-Rapidly Accelerated Fibrosarcoma (B-RAF) inhibitors that can paradoxically activate proto-oncogene (C-RAF) and the downstream kinase, mitogen-activated kinase kinase (MEK, also referred to as MAPKK). As described in the First Experimental Example, hyper-activation of MEK and its target extracellular signal-regulated kinase (ERK) have been identified as key drivers of Darier disease, which displays pathologic features identical to Grover disease. To model drug-induced Grover disease, live human keratinocytes were treated with dabrafenib or vemurafenib in this experimental example to inhibit BRAF and a fluorescent biosensor confirmed this activated ERK. Further, BRAF inhibitors were found to disrupt intercellular junctions in organotypic epidermis and compromised the integrity of keratinocyte sheets. Consistent with clinical data showing that MEK blockade prevents Grover disease in patients treated with B-RAF inhibitors, MEK inhibitors were demonstrated to suppress ERK activity and rescue cohesion of B-RAF-inhibited keratinocyte sheets. Finally, Grover disease biopsies were found to exhibit elevated ERK activation in lesional skin. In sum, the role of ERK hyperactivation in Grover disease was demonstrated in this experimental example, pointing to MEK inhibition as a therapeutic strategy.

### Methods

### Reagents

Inhibitors of MEK including trametinib (Cat. #62206), U0126 (Cat. #9903), PD98059 (Cat. #9900), PD184352 (Cat. #12147) plus inhibitors of B-RAF including dabrafenib (Cat. #91942) and vemurafenib (Cat. #17531) were from Cell Signaling Technology of Danvers, MA. Rabbit antibodies against phospho-ERK1/2 (D13.14.4E; Cat. #4370), pan-cadherin (Cat. #4068), plakoglobin (Cat. #75550) and mouse anti-ERK1/2 (L34F12; Cat. #4696) were from Cell Signaling Technology. Rabbit anti-KRT10 (Cat. #ab76318) was from Abcam of Cambridge, United Kingdom. Mouse anti-DSG1 (Cat. #sc-137164), anti-DSG2 (Cat. #sc-80663), anti-DSG3 (Cat. #sc-53487), anti-plakoglobin (Cat. #sc-514115), anti-GAPDH (Cat. #sc-47724), and anti-β-Actin (C4; Cat. #sc-47778) were from Santa Cruz Biotechnologies of Dallas, TX. Chicken anti-plakoglobin (#1408) was a gift from Dr. Kathleen Green (Northwestern University, Chicago, IL, USA). Secondary antibodies for fluorescent immunoblotting included IRDye 800CW goat anti-rabbit IgG (Cat. #926-32211) and IRDye 680RD goat anti-mouse IgG (Cat. #926-68070) from LI-COR Biosciences of Lincoln, NE. For immunofluorescent staining, secondary antibodies were from Thermo-Fisher of Waltham, MA: Goat anti-mouse IgG AlexaFluor-405 (Cat. #A31553), AlexaFluor-488 (Cat. #A11001), AlexaFluor-594 (Cat. #A11005), or AlexaFluor-633 (Cat. #A21050); goat anti-rabbit IgG AlexaFluor-405 (Cat. #A31556), AlexaFluor-488 (Cat. #A11008), AlexaFluor-594 (Cat. #A11012), or AlexaFluor-633 (Cat. #A21070). Hoechst 33342 was from Thermo-Fisher (Cat. #H1399). The ERK biosensor (pLenti-CMV-Puro-DEST-ERK-KTR-mClover; Cat. #59150) was from Addgene of Watertown, MA.

### Cell Culture

Normal human epidermal keratinocytes (NHEKs) were grown in Medium 154 with 0.07 mM CaCl₂ (M154; Thermo-Fisher Cat. #M154CF500) and 1× human keratinocyte growth supplement (Thermo-Fisher Cat. #S0015), and 1× gentamicin/amphotericin (Thermo-Fisher Cat. #R01510).

J2-3T3 immortalized murine fibroblasts were grown in complete Dulbecco's Modified Eagle Medium (DMEM) (Thermo-Fisher Cat. #11965092) supplemented with 10% fetal bovine serum (FBS) (Hyclone of Logan, UT, Fisher Scientific of Waltham, MA, Cat. #SH3039603), 2 mM GlutaMAX (Thermo-Fisher Cat. #35050061), 100 U/mL penicillin, and 100 μg/mL streptomycin.

All cell lines were maintained at 37° C. in 5% CO₂ in an air-jacketed, humidified incubator. Cells were grown on sterile cell culture dishes and passaged at sub-confluency using 0.25% Trypsin-EDTA (Thermo-Fisher Cat. #15400054).

### Organotypic Epidermal Culture

Human organotypic epidermal “raft cultures” were generated as described (Simpson C L, et al. Methods Mol Biol. 2010; 585: 127-46; Simpson C L, et al. Cell Rep. 2021; 34: 108689). Cultures were differentiated using E-medium, a 3:1 mixture of DMEM:Ham's F12 (Thermo-Fisher Cat. #11765054) with 10% FBS, 180 μM adenine (Sigma of Burlington, MA, Cat. #A2786), 0.4 μg/mL hydrocortisone (Sigma, Cat. #H0888), 5 μg/mL human insulin (Sigma Cat. #91077C), 0.1 nM cholera toxin (Sigma, Cat. #C8052), 5 μg/mL apo-transferrin (Sigma Cat. #T1147), and 1.36 ng/mL 3,3′,5-triiodo-L-thyronine (Sigma Cat. #T6397).

J2-3T3 fibroblasts were seeded into collagen matrix rafts within transwells (Corning of Corning, NY, Cat. #353091). For each raft, 1×10⁶ fibroblasts were resuspended in 1/10 the final volume of sterile filtered reconstitution buffer (1.1 g of NaHCO₃ plus 2.39 g of 4-(2-hydroxyethyl)-1-piperazineethanesulfonic acid (HEPES) in 50 mL 0.05 N NaOH), then 1/10 the final volume of 10×DMEM (Sigma-Aldrich of Burlington, MA, Cat. #D2429) was added. The cells were mixed thoroughly by pipetting, then high-concentration rat tail collagen I (Corning Cat. #CB354249) was added (4 mg/mL final concentration), along with sterile deionized water (diH₂O) to bring the solution up to the final volume. If necessary, 0.05 N NaOH was added dropwise to adjust the pH to 7 based on the phenol red indicator. The collagen-fibroblast slurry was mixed via inversion, then pipetted into a transwell insert placed within a deep 6-well cell culture plates (Corning Cat. #08-774-183). The rafts were polymerized at 37° C. for one hour, after which they were submerged in complete DMEM and incubated at 37° C. overnight.

Next, confluent keratinocyte cultures were trypsinized and resuspended in E-medium with epidermal growth factor (EGF) (5 ng/ml) to final concentration of 0.5×10⁶ cells/mL (2 mL per organotypic culture). The DMEM was aspirated from the upper and lower transwell chambers, then 2 mL (1×10⁶ cells) of the keratinocyte suspension were pipetted on top each raft. E-medium with EGF was added to the bottom transwell chamber to submerge the raft, and the cultures were incubated at 37° C. After 24 hours, E-medium was aspirated from the top and bottom chambers. An air-liquid interface was established to induce stratification by adding E-medium (without EGF) only to the bottom chamber to reach the bottom of the raft. Organotypic cultures were grown at 37° C. for up to 12 days. E-medium in the bottom chamber was replaced every other day. For drug treatments, inhibitors or vehicle control (dimethyl sulfoxide (DMSO)) were diluted in E-medium in the bottom chamber. Inhibitors were used at the following concentrations: Dabrafenib (1 μM), vemurafenib (10 μM), trametinib (1 μM). For histologic examination, the transwell was moved to a standard 6-well cell culture plate and submerged in 10% neutral-buffered formalin (Fisher Scientific Cat. #22-026-435) for at least 24 hours. Organotypic cultures were processed for histologic examination by the Experimental Histopathology Core of the Fred Hutchinson Cancer Center of Seattle, WA.

### Immunoblotting

NHEKs were seeded at a density of 1×10⁶ cells per well of 6-well cell culture dishes in M154. Upon reaching confluence, M154 was replaced with E-medium containing vehicle control (DMSO) or inhibitors at the following concentrations: Dabrafenib (1 μM), vemurafenib (10 μM), trametinib (1 μM). After 24 hours, whole-cell lysates were generated by washing cells once in phosphate-buffered saline (PBS) followed by lysis in urea sample buffer [8 M Urea, 60 mM Tris, 1% SDS, 10% glycerol, 5% β-mercaptoethanol, 0.0005% pyronin-Y, pH 6.8] for 10 min. Lysates were homogenized using a microtip probe sonicator (Fisher Scientific).

Whole-cell lysates were loaded onto NuPAGE 12% Bis-Tris Gels (Thermo-Fisher Cat. #NP0343BOX) and separated by electrophoresis using NuPAGE MES SDS Running Buffer (Thermo-Fisher Cat. #NP0002). Proteins were transferred onto Immobilon-FL membrane (Millipore of Burlington, MA, Cat. #IPFL85R) in transfer buffer (25 mM Tris, 192 mM glycine, 20% (v/v) methanol) at 50 volts (V) for 60 minutes. Membranes were blocked in Intercept tris-buffered saline (TBS) blocking buffer (LI-COR) for 60 minutes at room temperature. Membranes were probed overnight at 4° C. with the primary antibodies diluted in Intercept TBS blocking buffer (LI-COR). Blots were washed at least three times in 1×TBS containing 0.1% (v/v) Tween-20 (TBS-T), then incubated for 1 hour at room temperature with IRDye 8000W goat anti-rabbit IgG and/or IRDye 680RD goat anti-mouse IgG (LI-COR) diluted 1:10,000 in Intercept TBS blocking buffer. Blots were washed at least three times in TBS-T and proteins were visualized using an Odyssey M Imaging System (LI-COR).

### Fluorescent Immunocytochemistry

Keratinocytes were grown to confluency in 35 mm glass-bottom cell culture dishes (MatTek of Ashland, MA, Cat. #P35G-1.5-20-C). For staining keratins and desmosomal proteins, cells were fixed in ice-cold 100% methanol at −20° C. for 2 minutes, allowed to dry, then re-hydrated in PBS. For staining other proteins, cells were fixed in 4% paraformaldehyde for 10 minutes at 37° C. Fixed cells were then incubated with blocking buffer [0.5% (w/v) bovine serum albumin (BSA, Sigma), 10% (w/v) normal goat serum (NGS, Sigma) in PBS] for 30 min at 37° C. Cells were then washed with PBS. Primary antibodies were diluted in 0.5% (w/v) BSA in PBS and incubated on the cells overnight at 4° C. Primary antibody dilutions were as follows: chicken anti-plakoglobin (1:1000). Cells were washed three times with PBS, then incubated with species-specific secondary antibodies diluted at 1:300 (with or without Hoechst at 1:500) in 0.5% (w/v) BSA in PBS for 30 minutes at 37° C. Cells were washed three times with PBS, then held in PBS for imaging using spinning-disk confocal microscopy, as detailed below.

### Histologic Analysis and Tissue Procurement

Paraffin-embedded formalin-fixed tissue cross-sections of organotypic epidermis or skin biopsies were processed for histology and stained with hematoxylin and eosin (H&E) using standard methods. H&E-stained glass slides were imaged on an EVOS FL imaging system (Thermo-Fisher) using an EVOS 40× long working distance, achromatic, phase-contrast objective (Thermo-Fisher). Images were captured using the embedded high-sensitivity interline CCD color camera.

### Fluorescent Immunohistochemistry

Paraffin-embedded formalin-fixed tissue cross-sections on glass slides were incubated at 65° C. for 2 hours. Sections were prepared for staining by immersion in 3 baths of xylenes (Fisher) for 5 minutes each, followed by 3 baths of 95% ethanol for 5 minutes each, then 70% ethanol for 5 minutes, and finally 3 baths of PBS for 5 minutes each. Slides were then submerged in antigen retrieval solution [0.1 M sodium citrate (pH 6.0) with 0.05% (v/v) Tween-20] and heated to 95° C. for 15 minutes. Slides were allowed to cool to room temperature and then washed with PBS. Tissue sections were encircled with a hydrophobic barrier using a PAP pen. Tissue sections were incubated in blocking buffer [0.5% (w/v) BSA, 10% (v/v) NGS in PBS] for 30 minutes at 37° C. in a humidified chamber. Slides were washed in 3 baths of PBS for 5 minutes each, then incubated with primary antibodies diluted in 0.5% (w/v) BSA in PBS overnight at 4° C. in a humidified chamber. Primary antibody dilutions were as follows: rabbit anti-cytokeratin 10 (1:3000), mouse anti-desmoglein 1 (1:50), rabbit anti-phospho-ERK (1:400), mouse anti-ERK (1:400), or chicken anti-plakoglobin (1:1000). Slides were then washed in 3 baths of PBS for 5 min each and incubated with secondary antibodies diluted at 1:300 (with or without Hoechst at 1:500) in 0.5% (w/v) BSA in PBS for 60 minutes at 37° C. in a humidified chamber. Slides were washed in 3 baths of PBS for 5 minutes each and mounted in Prolong Gold (Thermo-Fisher Cat. #P36934) with a glass coverslip applied over the tissue sections. Slides were allowed to dry overnight prior to imaging by spinning-disk confocal microscopy, as below.

### Fluorescence Microscopy Imaging

Images were acquired on a Hamamatsu ORCA-FusionBT sCMOS camera (Hamamatsu of Hamamatsu City, Japan) using a Yokogawa W1 spinning-disk confocal (SDC) system (Yokogawa of Musashino, Tokyo, Japan) on a Nikon Ti2 microscope (Nikon of Minato City, Tokyo, Japan). Samples were illuminated using 405, 488, 561, and 640 nm laser excitation lines and fluorescence was detected using a 60×1.2 numerical aperture (NA) water objective (Nikon) with standard emission filters.

### ERK Biosensor Imaging

For live imaging of the ERK biosensor, NHEKs were transduced with ERK-KTR-mClover (Addgene Cat. #59150). HEK293FT cells were grown in complete DMEM, then transfected with 4 μg pLenti-CMV-PuroDEST-ERK-KTR-mClover DNA plus 12 μl FuGENE 6 (Promega of Madison, WI, Cat. #E2691) in 800 μL of Opti-MEM (Thermo-Fisher Cat. #31985070), which was added to the cells and left overnight. Lentiviral supernatants were collected the next day and polybrene (Sigma Cat. #H9268) was added at a concentration of 4 μg/mL. M154 was removed from NHEKs and replaced with viral medium for 1 hour at 37° C. Cells were then washed in PBS and placed back in their normal medium and expanded in culture.

ERK-KTR-mClover-transduced cells were seeded into 35 mm glass-bottom dishes in low calcium medium (0.31 mM) and grown to confluency. Cells were then exposed to high-calcium (1.3 mM) for 24 hours, then imaged by SDC microscopy. Using Fiji (Fiji software, https://github.com/fiji/fiji), the nuclear region was encircled with the polygon tool and the “Measure” function was used to calculate the mean fluorescence intensity; after using the “Cut” function to remove the nuclear region, then remaining cytoplasmic region was encircled with the polygon tool and the “Measure” function was used to calculate the mean fluorescence intensity. The ERK activity index was calculated as the ratio of the cytoplasmic integrated fluorescence intensity (active) to the nuclear integrated fluorescence intensity.

### Fluorescent Tissue Staining Quantification

Fluorescence images of immunostained tissue sections were captured by SDC microscopy as above and analyzed using Fiji. Quantification of fluorescence intensity was performed using non-visibly labeled images from immunostained tissue cross-sections. The region of interest (entire epidermis) was encircled with the polygon tool, then the Fiji “Measure” function was used to calculate the mean intensity of the fluorescence signal. The mean fluorescence intensity was averaged across multiple non-overlapping high-powered fields (HPF) and the values from control samples were normalized to an average value of 1.

### Monolayer Mechanical Dissociation Assay

Dispase-based mechanical dissociation assays were carried out as described (70). Keratinocytes were plated at a density of 1×10⁶ cells per well of 6-well cell culture dishes. Upon reaching confluence, the cells were switched into E-medium. Vehicle control (DMSO) or chemical inhibitors were added at the following concentrations: Dabrafenib (1 μM), vemurafenib (10 μM) trametinib (1 μM), U0126 (10 μM), or PD98059 (20 μM). After 24 h, monolayers were washed with PBS and then incubated with 500 μL dispase (5 U/ml) in Hank's balanced salt solution (Stemcell Technologies of Vancouver, Canada, Cat. #07913) for 30 minutes at 37° C. Next, 4.5 mL PBS was added to the wells and all liquid plus released monolayers were transferred into 15 mL conical tubes, which were placed together in a rack and inverted 1-10 times to induce mechanical stress. Monolayer fragments were transferred back into 6-well cell culture plates and imaged with a 12-megapixel digital camera. Fragments were counted in Fiji.

### Statistics

Statistical analyses were performed using Prism version 9 (GraphPad of La Jolla, CA), which was also used to generate graphs. Statistical parameters including sample size, definition of center, dispersion measures, and statistical tests are included in each figure legend. Datasets were tested for normality using the D'Agostino-Pearson test. The means of two normally distributed groups were compared using a two-tailed unpaired Student's t test. The means of more than two normally distributed groups were compared using a one-way ordinary ANOVA followed by P-value adjustment for multiple comparisons. P-values less than 0.05 were considered statistically significant. Exact P-values are included in each figure.

### Study Approval

Normal human epidermal keratinocytes (NHEKs) were cultured from de-identified neonatal foreskins procured by the Penn Skin Biology and Diseases Resource-based Center (SBDRC) (University of Pennsylvania of Philadelphia, PA) under a protocol (#808224) approved by the University of Pennsylvania Institutional Review Board (IRB). Tissue cross-sections from de-identified skin biopsies were obtained from a tissue bank by the Penn SBDRC under a protocol (#808225) approved by the University of Pennsylvania IRB. The use of de-identified tissues collected for clinical purposes that would otherwise be discarded was deemed exempt by the IRB for written informed consent.

### Results

### Sustained B-RAF Blockade Paradoxically Activates ERK in Human Epidermal Keratinocytes.

In patients treated chronically with systemic B-RAF inhibitors for malignancies driven by activating BRAF mutations, drug-induced Grover disease (GD) is a common cutaneous toxicity (Anforth R, et al. Lancet Oncol. 2013; 14: e11-8; Chu E Y, et al. J Am Acad Dermatol. 2012; 67: 1265-72). In these cells, B-RAF inhibitors cause compensatory upregulation of C-RAF, which leads to paradoxical activation of the mitogen-activated protein (MAP) kinase pathway via downstream kinases MEK and ERK; this was originally shown in human anaplastic carcinoma cells (Poulikakos P I, et al. Nature. 2010; 464: 427-30). Normal human epidermal keratinocytes (NHEKs), the cells that manifest GD pathology, were treated with either of the two B-RAF inhibitors that most often induce GD, dabrafenib (Dab) and vemurafenib (Vem).

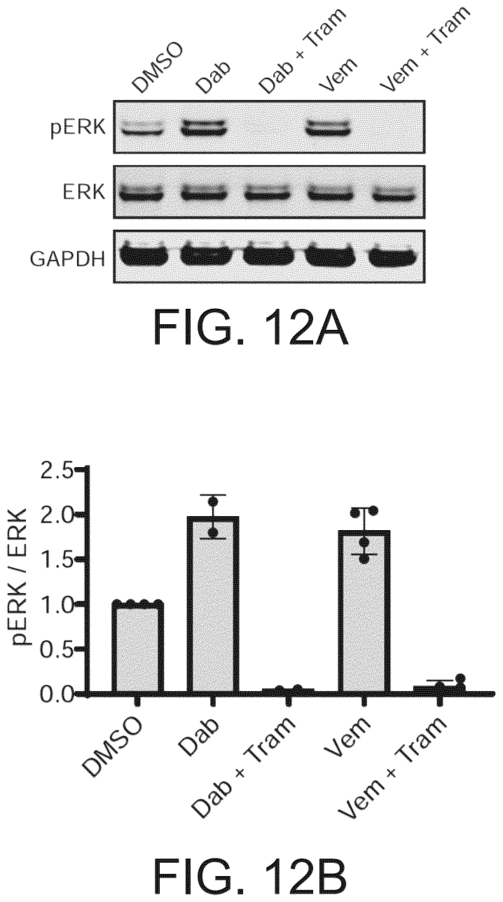

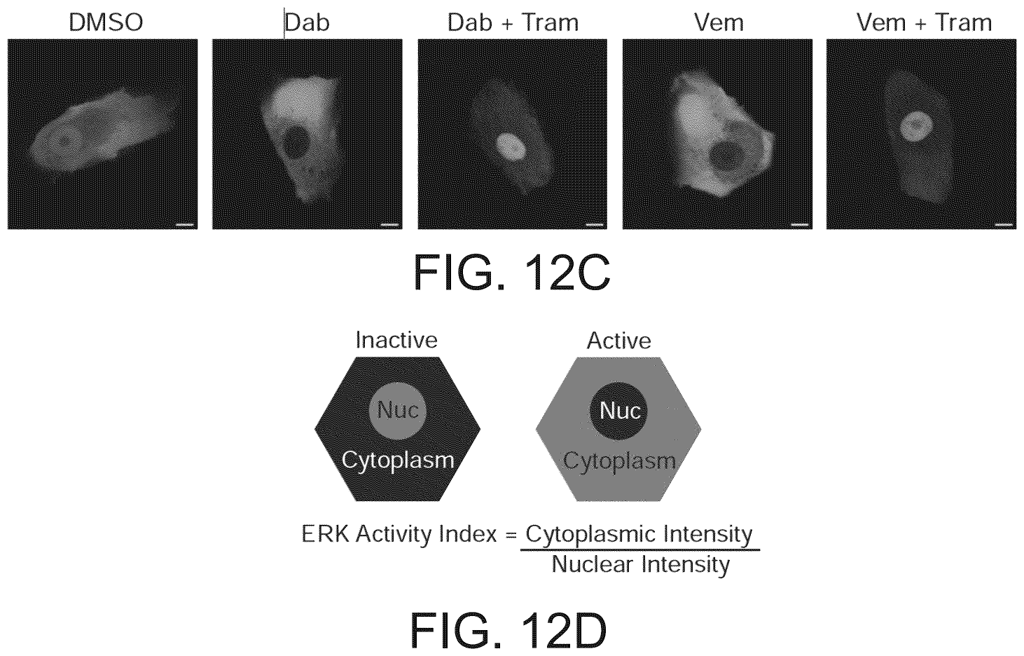

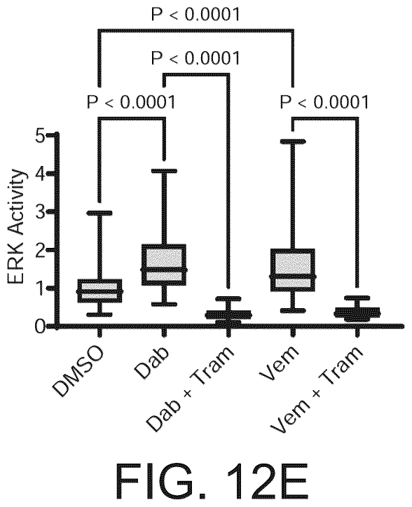

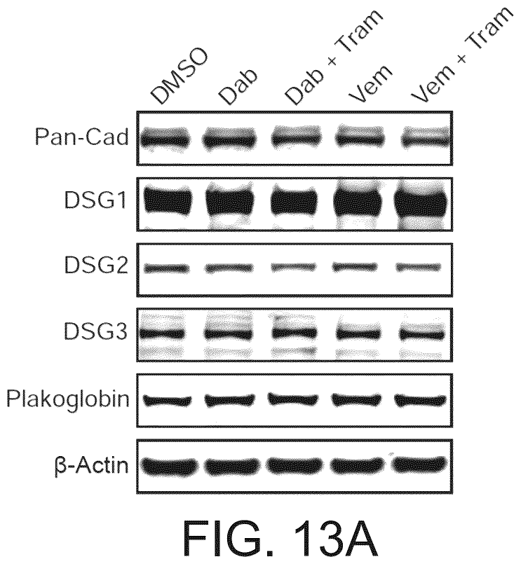

Treatment of keratinocytes with Dab or Vein was sufficient to increase the active phosphorylated form of ERK (pERK) compared to drug vehicle (dimethyl sulfoxide, DMSO) as quantified by Western blotting (WB) of NHEK lysates; ERK activation was completely dampened by using trametinib to inhibit MEK, the upstream kinase in the MAP kinase pathway (FIGS. 12A, 12B). This biochemical finding was confirmed using a validated fluorescent biosensor that has been engineered to shuttle out of or into the nucleus upon ERK activation or inactivation, respectively (de la Cova C, et al. Dev Cell. 2017; 42: 542-553 e4). In live NHEKs transduced with the ERK kinase translocation reporter linked to a green fluorescent protein (ERK-KTR-Clover), treatment with Dab or Vein induced ERK activation in a manner that could be reversed by inhibiting MEK (FIGS. 12C-12E). These findings confirm that NHEKs exhibit paradoxical activation of ERK upon sustained treatment with selective B-RAF inhibitors that have previously been linked to GD in clinical studies.

### B-RAF Inhibition Disrupts Desmosomal Protein Localization in Epidermal Keratinocytes.

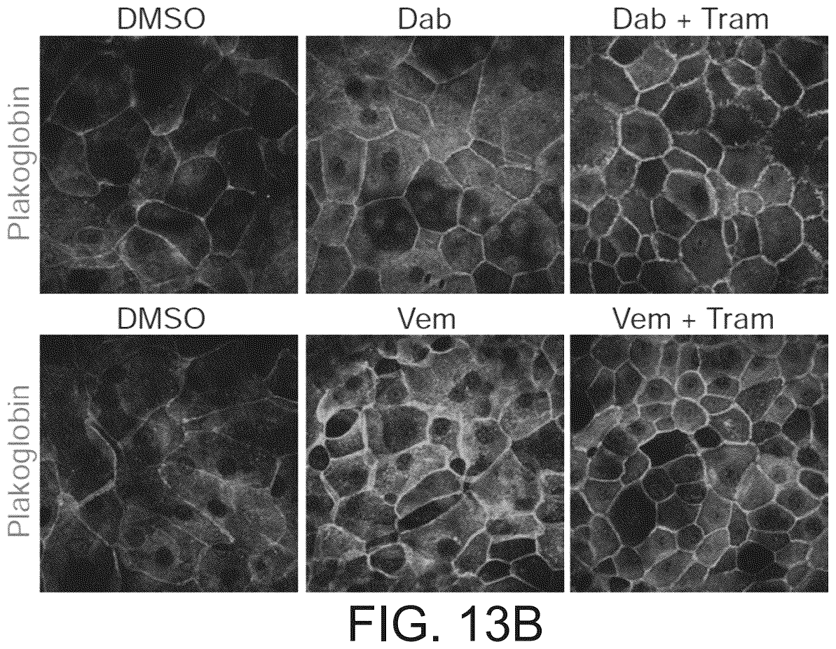

ERK is well known to regulate cell-cell adhesion and differentiation of keratinocytes (Harmon R M, et al. J Clin Invest. 2013; 123: 1556-70; Egu D T, et al. Front Immunol. 2019; 10: 2883; Hiratsuka T, et al. Proc Natl Acad Sci USA. 2020; 117:17796-17807; Khavari T A, Rinn J. Cell Cycle. 2007; 6: 2928-31), but this MAP kinase has not been directly linked to GD pathogenesis. To test if BRAF inhibitor-induced ERK activation impaired intercellular adhesion, the level and localization of cell-cell junction proteins in NHEKs was assessed. Compared to control keratinocytes treated with DMSO, the RAF inhibitors dabrafenib or vemurafenib did not appreciably alter protein levels of classical cadherins, desmosomal cadherins (DSG1, DSG2, DSG3) or their catenin binding partner plakoglobin (PG) (FIG. 13A). However, using immunofluorescence (ImF) microscopy, it was found that both dabrafenib and vemurafenib induced mislocalization of desmosomal proteins (FIG. 13B). Compared to DMSO-treated NHEKs, those treated with B-RAF inhibitors displayed a larger amount of PG in the cytoplasm rather than concentrated at intercellular borders, where it is needed to anchor keratin filaments and build strong intercellular adhesions between neighboring keratinocytes.

Using a mechanical dissociation assay validated for measuring intercellular adhesive strength mediated by desmosomes in keratinocyte sheets (Hudson T Y, et al. Methods Cell Biol. 2004; 78: 757-86), it was demonstrated that the disruption of desmosomal organization noted in B-RAF-inhibited NHEKs translated into marked weakening of intercellular adhesion. NHEK monolayers treated with either dabrafenib or vemurafenib exhibited an increase in the number of fragments generated upon mechanical stress, reflecting reduced intercellular adhesive strength (FIGS. 14A, 14B). Together, these results explain how B-RAF inhibitors can induce GD pathology through impaired localization of adhesive proteins, which weakens desmosomes to cause severing of cell-cell junctions (acantholysis) in epidermal keratinocytes that manifests as skin blistering.

### MEK Suppression Reverses B-RAF Inhibitor-Induced Weakening of Intercellular Adhesion.

Given that co-treatment of patients with MEK inhibitors prevented B-RAF inhibitor-induced GD (Carlos G, et al. JAMA Dermatol. 2015; 151: 1103-9), it was examined whether MEK inhibitors would rescue cell-cell junctions in Dab- or Vem-treated NHEKs as a model of drug-induced GD. Trametinib (Tram), a selective MEK inhibitor FDA-approved for BRAF mutant cancers (Flaherty K T, et al. N Engl J Med. 2012; 367:107-14; Roskoski R, Jr. Pharmacol Res. 2019; 142: 151-168), greatly enhanced localization of desmosomal proteins to cell-cell junctions in NHEKs treated with Dab or Vein (FIG. 13B).

This rescue of cell-cell junctions in NHEKs translated into an increase in intercellular adhesive strength. While NHEK monolayers readily fragmented upon Dab or Vein, co-treatment with Tram overcame the effect of B-RAF inhibitors, restoring the integrity of keratinocyte sheets to the level of control cultures treated with drug vehicle (FIGS. 14A, 14B). Two other MEK inhibitors (U0126, PD98059 (PD980)) were tested, which comparably rescued cell cohesion in NHEK monolayers treated with Dab (FIG. 14C). These data confirm the need to specifically dampen downstream ERK activity to reverse B-RAF inhibitor-induced activation of MEK and resultant disruption of desmosomal adhesion (FIG. 14D).

### B-RAF Inhibitors Reversibly Disrupt Cell-Cell Junctions in Organotypic Human Epidermis.

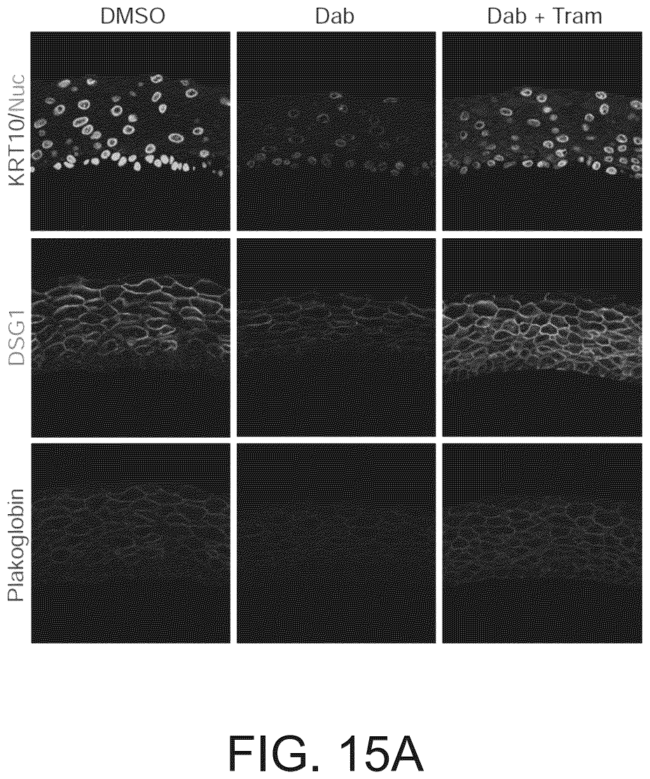

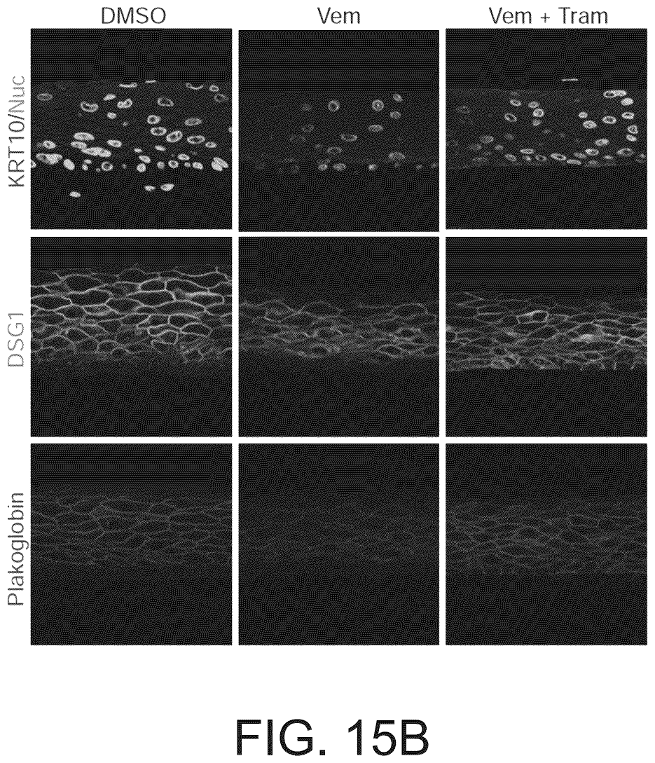

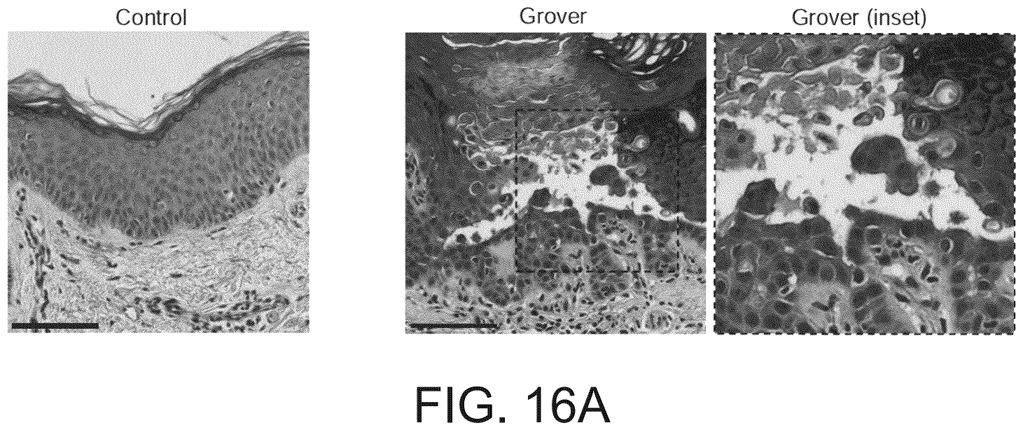

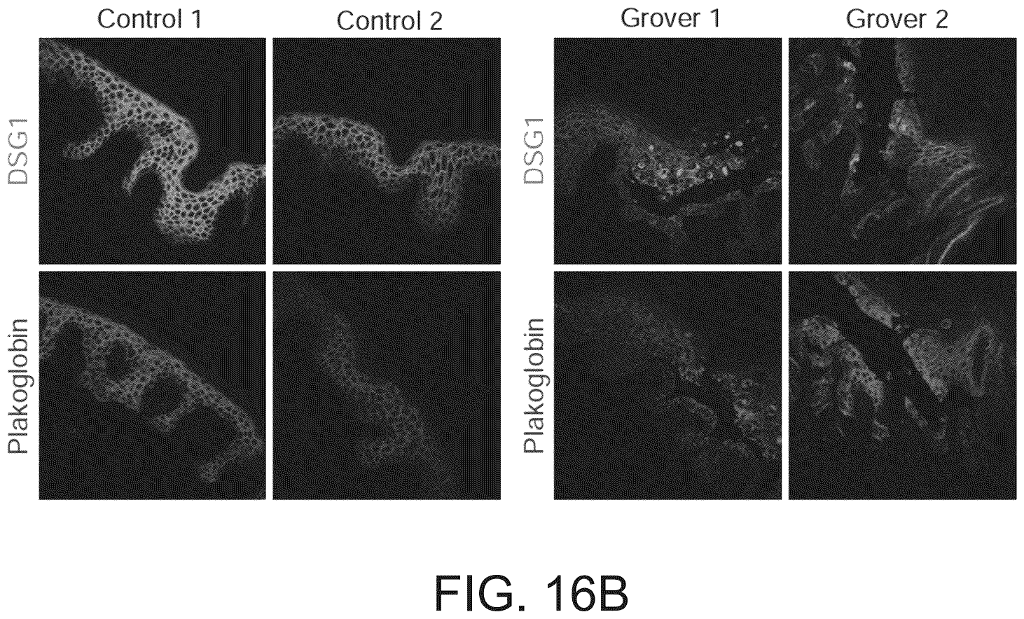

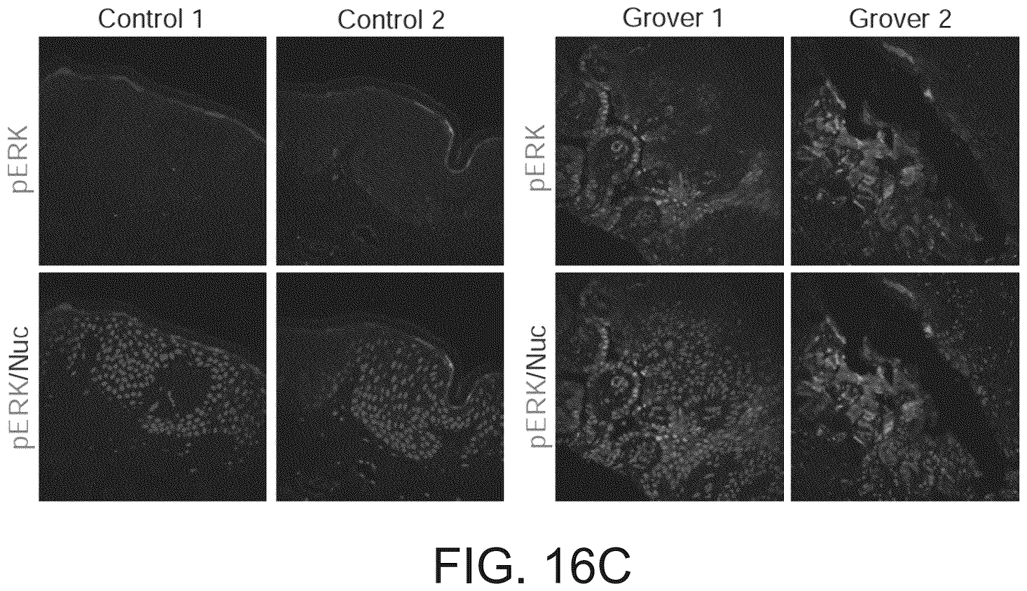

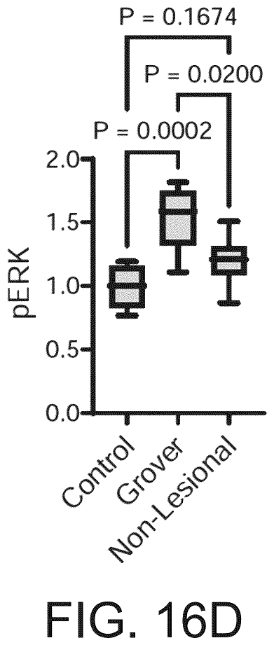

To test the effects of B-RAF inhibitors in a 3-D human tissue context, NHEKs were grown as organotypic epidermis, which replicates fully differentiated epidermal morphology within a week (Simpson C L, et al. Methods Mol Biol. 2010; 585: 127-46). After treating mature epidermal cultures with Dab or Vein for 48 hours, areas of complete acantholysis in hematoxylin and eosin (H&E) stained tissue sections were not found, which is likely due to the lack of shear mechanical forces on the in vitro epidermal tissue model. However, immunostaining of desmosomal components in organotypic epidermis treated with Dab or Vein revealed reduced localization of plakoglobin to cell-cell junctions especially in the lower keratinocyte layers (FIGS. 15A, 15B), where acantholysis most commonly occurs in GD (FIG. 16A). These data are also consistent with prior work that showed mislocalization of desmosomal proteins, including plakoglobin, in GD skin biopsies (Hashimoto K, et al. J Cutan Pathol. 1995; 22: 488-501). To test if MEK inhibition was sufficient to prevent B-RAF inhibitor-induced disruption of desmosomal components, organotypic epidermis was treated with Dab or Vein in combination with Tram to inhibit MEK. Tram rescued plakoglobin localization at cell-cell borders despite Dab or Vein treatment (FIGS. 15A, 151B). Consistent with prior work showing MEK inhibitors increase DSG1 and KRT10 expression in keratinocytes (Zaver S A, et al. JCI Insight. 2023), Tram also augmented DSG1 and KRT10 levels in B-RAF-inhibited epidermal cultures (FIGS. 15A, 15B), which could enhance their mechanical integrity.

Biopsies of Grover Disease Exhibit ERK Hyperactivation Along with Desmosomal Disruption.

Since GD is not a genetic disorder that can be easily replicated using knockout cells or mice, a model of drug-induced GD was established, as described above, using B-RAF inhibitors that have been robustly linked in clinical studies to inducing specific GD pathology in patients. Based on results disclosed herein showing that B-RAF inhibition causes over-activation of ERK in human keratinocytes, it was examined whether same mechanism could drive the pathology of idiopathic GD (FIG. 16A). To test this, 17 de-identified skin biopsies from a cohort of patients with biopsy-confirmed GD were obtained and immunostaining for both intercellular junctions and pERK was performed.

Compared to biopsies of normal control skin, staining of the key epidermal desmosome components in GD biopsies revealed clear disruption of cell-cell junctions. Within lesional acantholytic areas, DSG1 and PG were nearly completely internalized within the cytoplasm and collapsed around the nucleus of epidermal keratinocytes rather than at intercellular borders (FIG. 16B) consistent with the findings of prior studies of GD (Hashimoto K, et al. J Cutan Pathol. 1995; 22: 488-501). A concomitant increase in ERK phosphorylation was found in GD lesions (FIGS. 16C, 16D). Together, these results indicate overactivation of the MAP kinase pathway via ERK in the pathogenesis of both drug-induced and idiopathic GD and underscore the potential therapeutic value of MEK inhibitors (some of which are already FDA-approved) for treating this skin disorder (FIG. 14D).

### Discussion

Molecular therapies have completely changed the treatment landscape and prognosis for malignances driven by specific mutations, such as BRAF V600E in melanoma (Flaherty K T. Clin Exp Metastasis. 2012; 29: 841-6). Despite being highly selective for their targets, inhibitors of the MAP kinase signaling pathway have had unanticipated side effects, including frequent cutaneous adverse events that reduced drug safety and tolerability, impaired quality of life, and even disqualified patients from trials (Belum V R, et al. Curr Oncol Rep. 2013; 15: 249-59; Mondaca S, et al. JCO Precis Oncol. 2018; 2018). As a silver lining, off-target effects of a drug can provide insight into the pathogenesis of other diseases. In clinical trials of B-RAF inhibitor monotherapy for cancer, investigators reported an unexpectedly common skin eruption with biopsy features diagnostic of GD (Anforth R, et al. Lancet Oncol. 2013; 14: e11-8; Chu E Y, et al. J Am Acad Dermatol. 2012; 67: 1265-72); subsequent analysis indicated GD was seen in 42.9% and 38.9% of patients treated with either Dab or Vein, respectively (Carlos G, et al. JAMA Dermatol. 2015; 151: 1103-9). However, it was unclear how B-RAF blockade replicated the specific pathologic findings of a rare blistering disorder that had not previously been linked to this signaling pathway. Multiple in vitro assays were used in this example to demonstrate that B-RAF inhibitors are sufficient to increase activation of ERK, which disrupted desmosomal adhesion in human epidermal keratinocytes and organotypic epidermis, thus explaining the loss of tissue integrity typical of GD pathology.

The pathologic features of GD can be identical to Darier disease (See S H C, et al. J Cutan Pathol. 2019; 46: 6-15; Chalet M, et al. Arch Dermatol. 1977; 113: 431-5; Hashimoto K, et al. J Cutan Pathol. 1995; 22: 488-501), a genetic disorder linked to mutation of a calcium ATPase (SERCA2) embedded in the endoplasmic reticulum (Seli D, et al. JAMA Dermatol. 2023). Up to now, it remained unclear why these two disorders would exhibit such similar findings in biopsies that pathologists cannot consistently distinguish them. The in vitro model of Darier disease described in the First Experimental Example demonstrated that deficiency or chemical inhibition of SERCA2 induced hyperactivation of ERK (Zaver S A, et al. JCI Insight. 2023), which is reported here as a driver of GD. Further linking these disorders with distinct origins (inherited vs. acquired), some cases of GD were recently found to harbor acquired mutations in the ATP2A2 gene encoding SERCA2, a major regulator of calcium, which can activate ERK signaling in keratinocytes (Schmidt M, et al. J Biol Chem. 2000; 275: 41011-7). The convergence of these two disorders upon the same signaling pathway explains the similarity of their pathologic features. Moreover, recent RNA sequencing of skin biopsies from Grover and Darier disease identified an overlapping transcriptional signature that pointed to dysregulation of serum response factor and the actin cytoskeleton (Roth-Carter Q R, et al. JCI Insight. 2023; 8), both of which are modulated by MEK and ERK (Pagel J I, Deindl E. Indian J Biochem Biophys. 2011; 48: 226-35; Assoian R K, Klein E A. Trends Cell Biol. 2008; 18: 347-52; Pullikuth A K, Catling A D. Cell Signal. 2007; 19: 1621-32).

While molecular therapies have revolutionized the treatment of common inflammatory dermatologic conditions using monoclonal antibodies against cytokines or selective kinase inhibitors (Silverberg N. Clin Dermatol. 2020; 38: 462-466; Hill C R, Theos A. Dermatol Clin. 2019; 37: 229-239; David E, et al. Clin Exp Allergy. 2023; 53: 156-172; Lin C P, et al. Curr Opin Pharmacol. 2022; 67: 102292), targeted therapeutics remain elusive for rare blistering disorders. The findings described herein indicate that MEK inhibitors, which are FDA-approved for oral administration for multiple BRAF-driven cancers (Flaherty K T, et al. N Engl J Med. 2012; 367:107-14; Ram T, et al. RSC Med Chem. 2023; 14:1837-1857; Bouffet E, et al. N Engl J Med. 2023; 389: 1108-1120; Wen P Y, et al. Lancet Oncol. 2022; 23: 53-64; Subbiah V, et al. J Clin Oncol. 2018; 36: 7-13; Planchard D, et al. Lancet Oncol. 2017; 18: 1307-1316), could be therapeutic for GD as well as Darier disease. Moreover, MEK inhibitors can be delivered topically as shown by successful use of compounded trametinib to treat a cutaneous histiocyte proliferation driven by MAP kinase overactivation (Fay C J, et al. JAAD Case Rep. 2023; 39: 74-77). Topical MEK inhibition is likely to be even more feasible for blistering disorders like GD given their pathology lies in the epidermis and topical delivery could also obviate side effects from systemic use (Carlos G, et al. JAMA Dermatol. 2015; 151: 1103-9; Abdel-Rahman O, et al. Future Oncol. 2015; 11:3307-19; Anforth R, et al. Australas J Dermatol. 2014; 55: 250-4).

The results described herein indicate ERK as a driver of GD and demonstrate that MEK inhibition can restore desmosomal organization and rescue intercellular adhesion in cellular and organotypic models of drug-induced GD. Data from multiple clinical trials revealed that adding trametinib to RAF inhibitors eliminated drug-induced GD (Carlos G, et al. JAMA Dermatol. 2015; 151: 1103-9).

### Example Clauses

1. A method of treating a subject having a skin fragility disease, the method including administering a therapeutically effective amount of a mitogen-activated protein kinase kinase (MEK) inhibitor, thereby treating the subject.

2. The method of clause 1, wherein the skin fragility disease is a skin blistering disease; and or
wherein the skin fragility disease is associated with dysregulation of at least one of sarcoplasmic/endoplasmic reticulum Ca2+ transporting 2 (SERCA2), ATPase SERCA2 (ATP2A2), ATPase secretory pathway Ca2+ transporting 1 (ATP2C1), secretory pathway Ca2+ ATPase pump type 1 (SPCA1), MEK, extracellular signal-regulated kinase (ERK), phosphorylated ERK (pERK), a desmosome component, proto-oncogene B-Rapidly Accelerated Fibrosarcoma (B-RAF), proto-oncogene C-RAF (C-RAF), a keratin, plakoglobin, desmoplakin, or plakophilin.

3. The method of clauses 1 or 2, wherein the skin fragility disease is Darier disease, Grover disease, Hailey-Hailey disease, pemphigus, epidermolytic ichthyosis, epidermolysis bullosa simplex, keratoderma, palmoplantar keratoderma, or pachyonychia congenita.

4. The method of any of clauses 1-3, wherein the skin fragility disease is associated with administration of a B-RAF inhibitor to the subject.

5. The method of any of clauses 1-4, wherein the administering includes intravenous injection, intradermal injection, intramuscular injection, oral administration, subcutaneous administration, or topical administration.

6. The method of any of clauses 1-5, wherein the therapeutically effective amount is in a range of about 0.1 microgram per kilogram per day to about 2 milligram per kilogram per day.

7. The method of any of clauses 1-6 wherein the MEK inhibitor includes trametinib, cobimetinib, binimetinib, refametinib, selumetinib, U0126, PD98059, or PD184352.

8. The method of any of clauses 1-7, further including:
based on the administering, determining that a level of ERK activity or phosphorylation in the subject has decreased.

9. The method of any of clauses 1-8, further including:
predicting that the subject has the skin fragility disease.

10. The method of clause 9, wherein the predicting includes:
obtaining a sample derived from the subject;
determining a level of at least one of SERCA2, ATP2A2, SPCA1, ATP2C1, keratin (KRT) 1, KRT2, KRT5, KRT10, KRT14, KRT16, KRT17, desmoglein (DSG) 1, DSG2, DSG3, DSG4, desmocollin (DSC) 1, DSC2, DSC3, plakoglobin, desmoplakin, plakophilin, pERK, ERK activity, B-RAF activity, C-RAF activity, or MEK activity in the sample;
comparing the level of at least one of SERCA2, ATP2A2, SPCA1, ATP2C1, KRT1, KRT2, KRT5, KRT10, KRT14, KRT16, KRT17, DSG1, DSG2, DSG3, DSG4, DSC1, DSC2, DSC3, plakoglobin, desmoplakin, plakophilin, pERK, ERK activity, B-RAF activity, C-RAF activity, or MEK activity to a reference level; and
based on the comparing, predicting that the subject has the skin fragility disease.

11. A topical formulation for treating a skin fragility disease, the topical formulation including:
a therapeutically effective amount of a MEK inhibitor; and
a pharmaceutically acceptable carrier.

12. The topical formulation of clause 11, wherein the skin fragility disease is a skin blistering disease; and/or
wherein the skin fragility disease is associated with dysregulation of at least one of SERCA2, ATP2A2, ATP2C1, SPCA1, MEK, ERK, pERK, a desmosome component, B-RAF, C-RAF, a keratin, plakoglobin, desmoplakin, or plakophilin.

13. The topical formulation of clauses 11 or 12, wherein the skin fragility disease is Darier disease, Grover disease, Hailey-Hailey disease, pemphigus, epidermolytic ichthyosis, epidermolysis bullosa simplex, keratoderma, palmoplantar keratoderma, or pachyonychia congenita.

14. The topical formulation of any of clauses 11-13, wherein the therapeutically effective amount provides a prophylactic or therapeutic treatment for the skin fragility disease.

15. The topical formulation of any of clauses 11-14, wherein the MEK inhibitor includes trametinib, cobimetinib, binimetinib, refametinib, selumetinib, U0126, PD98059, or PD184352.

16. The topical formulation of any of clauses 11-15, wherein the topical formulation is an ointment, a paste, a cream, a lotion, a gel, a powder, a solution, a spray, an inhalant, a patch, a suspension, an emulsion, a crystalline form, an oil, a plaster, a liposome, a microemulsion, or a buffered solution; and/or
wherein the topical formulation is incorporated into a bandage or a patch.

17. The topical formulation of any of clauses 11-16, wherein the pharmaceutically acceptable carrier includes an excipient, the excipient including at least one of an alcohol, a quaternary amine, an organic acid, a paraben, a phenol, ascorbic acid, an ascorbic acid ester, sodium bisulfite, butylated hydroxytoluene, butylated hydroxyanisole, a tocopherol, a chelating agent, glycerine, sorbitol, polyethylene glycol, urea, propylene glycol, citric buffer, hydrochloric buffer, lactic acid buffer, a quaternary ammonium chloride, a cyclodextrin, benzyl benzoate, lecithin, a polysorbate, vitamin E oil, allantoin, dimethicone, glycerin, petrolatum, or zinc oxide.

18. The topical formulation of any of clauses 11-17, further including a topical penetration enhancer, the penetration enhancer including at least one of a triglyceride, an aloe composition, ethyl alcohol, isopropyl alcohol, octylphenol polyethylene glycol, oleic acid, polyethylene glycol 400, propylene glycol, N-decylmethylsulfoxide, a fatty acid ester, or N-methylpyrrolidone.

19. A method including administering the topical formulation of clause 11 to a subject in need thereof.

20. The method of clause 19, wherein the administering includes applying the topical formulation to a skin lesion of the subject.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Variants of the protein, nucleic acid, and gene sequences disclosed herein also include sequences with at least 70% sequence identity, 80% sequence identity, 85% sequence identity, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A method of treating a subject having a skin fragility disease, the method comprising administering a therapeutically effective amount of a mitogen-activated protein kinase kinase (MEK) inhibitor, thereby treating the subject.

2. The method of claim 1, wherein the skin fragility disease is a skin blistering disease; and/or
wherein the skin fragility disease is associated with dysregulation of at least one of sarcoplasmic/endoplasmic reticulum Ca2+ transporting 2 (SERCA2), ATPase SERCA2 (ATP2A2), ATPase secretory pathway Ca2+ transporting 1 (ATP2C1), secretory pathway Ca2+ ATPase pump type 1 (SPCA1), MEK, extracellular signal-regulated kinase (ERK), phosphorylated ERK (pERK), a desmosome component, proto-oncogene B-Rapidly Accelerated Fibrosarcoma (B-RAF), proto-oncogene C-RAF (C-RAF), a keratin, plakoglobin, desmoplakin, or plakophilin.

3. The method of claim 1, wherein the skin fragility disease is Darier disease, Grover disease, Hailey-Hailey disease, pemphigus, epidermolytic ichthyosis, epidermolysis bullosa simplex, keratoderma, palmoplantar keratoderma, or pachyonychia congenita.

4. The method of claim 1, wherein the skin fragility disease is associated with administration of a B-RAF inhibitor to the subject.

5. The method of claim 1, wherein the administering comprises intravenous injection, intradermal injection, intramuscular injection, oral administration, subcutaneous administration, or topical administration.

6. The method of claim 1, wherein the therapeutically effective amount is in a range of about 0.1 microgram per kilogram per day to about 2 milligram per kilogram per day.

7. The method of claim 1, wherein the MEK inhibitor comprises trametinib, cobimetinib, binimetinib, refametinib, selumetinib, U0126, PD98059, or PD184352.

8. The method of claim 1, further comprising:
based on the administering, determining that a level of ERK activity or phosphorylation in the subject has decreased.

9. The method of claim 1, further comprising:
predicting that the subject has the skin fragility disease.

10. The method of claim 9, wherein the predicting comprises:
obtaining a sample derived from the subject;
determining a level of at least one of SERCA2, ATP2A2, SPCA1, ATP2C1, keratin (KRT) 1, KRT2, KRT5, KRT10, KRT14, KRT16, KRT17, desmoglein (DSG) 1, DSG2, DSG3, DSG4, desmocollin (DSC) 1, DSC2, DSC3, plakoglobin, desmoplakin, plakophilin, pERK, ERK activity, B-RAF activity, C-RAF activity, or MEK activity in the sample;
comparing the level of at least one of SERCA2, ATP2A2, SPCA1, ATP2C1, KRT1, KRT2, KRT5, KRT10, KRT14, KRT16, KRT17, DSG1, DSG2, DSG3, DSG4, DSC1, DSC2, DSC3, plakoglobin, desmoplakin, plakophilin, pERK, ERK activity, B-RAF activity, C-RAF activity, or MEK activity to a reference level; and
based on the comparing, predicting that the subject has the skin fragility disease.

11. A topical formulation for treating a skin fragility disease, the topical formulation comprising:
a therapeutically effective amount of a MEK inhibitor; and
a pharmaceutically acceptable carrier.

12. The topical formulation of claim 11, wherein the skin fragility disease is a skin blistering disease; and/or
wherein the skin fragility disease is associated with dysregulation of at least one of SERCA2, ATP2A2, ATP2C1, SPCA1, MEK, ERK, pERK, a desmosome component, B-RAF, C-RAF, a keratin, plakoglobin, desmoplakin, or plakophilin.

13. The topical formulation of claim 11, wherein the skin fragility disease is Darier disease, Grover disease, Hailey-Hailey disease, pemphigus, epidermolytic ichthyosis, epidermolysis bullosa simplex, keratoderma, palmoplantar keratoderma, or pachyonychia congenita.

14. The topical formulation of claim 11, wherein the therapeutically effective amount provides a prophylactic or therapeutic treatment for the skin fragility disease.

15. The topical formulation of claim 11, wherein the MEK inhibitor comprises trametinib, cobimetinib, binimetinib, refametinib, selumetinib, U0126, PD98059, or PD184352.

16. The topical formulation of claim 11, wherein the topical formulation is an ointment, a paste, a cream, a lotion, a gel, a powder, a solution, a spray, an inhalant, a patch, a suspension, an emulsion, a crystalline form, an oil, a plaster, a liposome, a microemulsion, or a buffered solution; and/or
wherein the topical formulation is incorporated into a bandage or a patch.

17. The topical formulation of claim 11, wherein the pharmaceutically acceptable carrier comprises an excipient, the excipient comprising at least one of an alcohol, a quaternary amine, an organic acid, a paraben, a phenol, ascorbic acid, an ascorbic acid ester, sodium bisulfite, butylated hydroxytoluene, butylated hydroxyanisole, a tocopherol, a chelating agent, glycerine, sorbitol, polyethylene glycol, urea, propylene glycol, citric buffer, hydrochloric buffer, lactic acid buffer, a quaternary ammonium chloride, a cyclodextrin, benzyl benzoate, lecithin, a polysorbate, vitamin E oil, allantoin, dimethicone, glycerin, petrolatum, or zinc oxide.

18. The topical formulation of claim 11, further comprising a topical penetration enhancer, the topical penetration enhancer comprising at least one of a triglyceride, an aloe composition, ethyl alcohol, isopropyl alcohol, octylphenol polyethylene glycol, oleic acid, polyethylene glycol 400, propylene glycol, N-decylmethylsulfoxide, a fatty acid ester, or N-methylpyrrolidone.

19. A method comprising administering the topical formulation of claim 11 to a subject in need thereof.

20. The method of claim 19, wherein the administering comprises applying the topical formulation to a skin lesion of the subject.


SEQUENCE LISTING (2 sequences)

SEQ ID NO: 1 - DNA, 20 bp, synthetic construct
  source 1..20: mol_type = other DNA
gttttggcttggtttgaaga

SEQ ID NO: 2 - DNA, 20 bp, synthetic construct
  source 1..20: mol_type = other DNA
gtattccgtgggtgaacggg